(19) United States
(12) Patent Application Publication
(10) Pub. No.: US 2022/0088146 A1
(43) Pub. Date: Mar. 24, 2022

(54) Melanocortin Receptor-Specific Peptide Formulations and Methods for Gastrointestinal Tract-Specific Delivery

(71) Applicant: Palatin Technologies, Inc., Cranbury, NJ (US)

(72) Inventors: John Harold Dodd, Spring Mills, PA (US); Stephen Kwaku Dordunoo, Rosedale, MD (US)

(21) Appl. No.: 17/028,437

(22) Filed: Sep. 22, 2020

Related U.S. Application Data
(63) Continuation of application No. PCT/US19/23575, filed on Mar. 22, 2019.
(60) Provisional application No. 62/647,000, filed on Mar. 23, 2018.

(51) Int. Cl. A61K 38/22; A61K 9/00; A61K 9/48

(52) U.S. Cl. CPC A61K 38/22; A61K 9/0053; A61K 9/4891; A61K 9/4866; A61K 9/4808

(57) ABSTRACT

Formulations, compositions and methods for delivery of melanocortin receptor-specific peptides, particularly cyclic peptides selective and specific for the melanocortin-1 receptor, to the lumen of the gastrointestinal tract for treatment of melanocortin receptor-mediated or responsive diseases, indications, conditions and syndromes of the gastrointestinal tract.

### CROSS-REFERENCE TO RELATED APPLICATIONS

This application is a continuation application of International Application No. PCT/US2019/023575, published as International Publication No. WO 2019/183472, entitled “Melanocortin Receptor-Specific Peptide Formulations and Methods for Gastrointestinal Tract-Specific Delivery”, filed on Mar. 22, 2019, which in turn claimed priority to and the benefit of the filing of U.S. Provisional Patent Application Ser. No. 62/647,000 entitled “Melanocortin Receptor-Specific Peptide Formulations and Methods for Gastrointestinal Tract-Specific Delivery”, filed Mar. 23, 2018, and the specification and claims thereof are incorporated herein by reference.

### SEQUENCE LISTING

The instant application contains a Sequence Listing which has been submitted electronically in ASCII format and is hereby incorporated by reference in its entirety.

### BACKGROUND OF THE INVENTION

### Field of the Invention (Technical Field)

The present invention relates to uses of melanocortin receptor-specific peptides, particularly cyclic peptides selective and specific for the melanocortin-1 receptor, and methods, compositions and formulations comprising such peptides, for gastrointestinal tract-specific delivery, including colon-specific delivery, for treatment of melanocortin receptor-mediated or responsive diseases, indications, conditions and syndromes, including melanocortin-1 receptor-mediated or responsive diseases, indications, conditions and syndromes.

### Description of Related Art

A family of melanocortin receptor types and subtypes has been identified. Receptor types include the melanocortin-1 receptor (MC1r), commonly known to be expressed in normal human melanocytes and on melanoma cells, but which is also reported to be expressed in various other cells, including those involved in immune responses, such as monocytes, neutrophils, lymphocytes, dendritic cells, natural killer (NK) cells and endothelial cells. See generally, Kang, L., et al., “A selective small molecule agonist of melanocortin-1 receptor inhibits lipopolysaccharide-induced cytokine accumulation and leukocyte infiltration in mice,” J. Leuk. Biol. 80:897-904 (2006), and references cited therein. A variety of human MC1r subtypes and variants are known, including those disclosed in U.S. Pat. Nos. 6,693,184 and 7,115,393. In addition to MC1r, other melanocortin receptor types include melanocortin-2 receptor (MC2r) for ACTH (adrenocorticotropin), expressed in cells of the adrenal gland, melanocortin-3 receptors (MC3r) and melanocortin-4 receptors (MC4r), expressed primarily in cells in the hypothalamus, mid-brain and brainstem as well as peripheral tissues, and melanocortin-5 receptor (MC5r), expressed in a wide distribution of peripheral tissues.

Highly selective and specific MC1r agonist peptides are known, including the cyclic peptides disclosed in U.S. Pat. Nos. 9,447,148, 8,877,890 and 8,492,517 and the linear peptides disclosed in U.S. Pat. Nos. 9,580,466 and 8,933,194.

There are a number of inflammatory bowel diseases (IBD) known, including both ulcerative colitis (UC) and Crohn's disease. Both diseases are chronic and relapsing/remitting IBDs of the gastrointestinal (GI) tract. The regions of the GI tract that are most often affected by Crohn's disease are the small intestine and large intestine, also called the colon, and including the rectum, but it is known that Crohn's disease can affect the entire GI tract from the mouth to the anus. UC commonly affects the large intestine, comprising the colon. Common symptoms of the diseases include diarrhea, abdominal pain, rectal bleeding and weight loss. Additionally Crohn's disease may include intestinal abscesses, fistula, an abnormal passage leading from one portion of the intestine to another and permitting passage of fluids or secretions, and intestinal obstructions.

It is known that MC1r are upregulated in certain experimental colitis animal models and expressed on the cell surface of intestinal epithelia. Maaser C., et al. Crucial role of the melanocortin receptor MC1R in experimental colitis. Gut. 2006; 55(10):1415-1422. However, heretofore use of MC1r-specific compounds for treatment of UC, Crohn's disease or IBD has been limited to systemic routes of administration, such as disclosed in International Publication Number WO 2016/066702, PCT/EP2015/075019.

Notwithstanding the intense scientific and pharmaceutical interest in melanocortin receptor-specific peptides, there remains a need for highly selective and specific MC1r agonist peptides for use in pharmaceutical applications, and formulations and methods of delivering MC1r agonist peptides to a targeted site, such as within the lumen of the colon. It is against this background that the present invention was made.

### BRIEF SUMMARY OF THE INVENTION

In one aspect, the invention provides a lower gastrointestinal (GI) tract release pharmaceutical formulation comprising a melanocortin receptor-specific peptide or a pharmaceutically acceptable salt thereof disposed within a particle matrix, such as a microparticle matrix, comprising at least one delayed release polymer. In the formulation, the delayed release polymer may be a pH-dependent release polymer. The peptide or pharmaceutically acceptable salt thereof may be admixed within the particle matrix, thereby forming an admixture of the particle matrix and the peptide or pharmaceutically acceptable salt thereof. The admixture of the particle matrix and the peptide or pharmaceutically acceptable salt thereof may be disposed within an aqueous soluble capsule, which may be a gelatin capsule, which capsule may further comprise at least one of a seal coating and an enteric coating. Alternatively, the admixture of the particle matrix and the peptide or pharmaceutically acceptable salt thereof may be formed into a tablet, and the tablet may further comprise at least one of a seal coating and an enteric coating.

The at least one delayed release polymer may include a pH-dependent release polymer, optionally comprising pH-sensitive methyl methacrylate/methacrylic copolymers, such as copolymers selected from the group consisting of Eudragit® L100-55, Eudragit® S100 and Eudragit® FS30D. The Eudragit® L100-55, Eudragit® S100 and Eudragit® FS30D copolymers may be present in a weight-to-weight ratio of L100-55 to S100 to FS30D selected from the group consisting of about 6:6:1, or about 6.2:6.2:1 or about 23.25:23:3.75.

The melanocortin receptor-specific peptide or a pharmaceutically acceptable salt thereof in the formulation may be a MC1r-specific peptide or a pharmaceutically acceptable salt thereof. The MC1r-specific peptide or a pharmaceutically acceptable salt thereof may have a functional EC₅₀ value at the MC1r of less than about one nM, and may further have a functional EC₅₀ value at the MC4r at least one hundred times the functional EC₅₀ value at MC1r. In one aspect, the MC1r-specific peptide or a pharmaceutically acceptable salt thereof has a functional EC₅₀ value at the MC4r of at least about 500 nM. In another aspect, the MC1r-specific peptide or a pharmaceutically acceptable salt thereof may be functionally inactive at the MC2r, the MC3r and the MC5r.

In one aspect, the delayed release polymer releases at least a portion of the MC1r-specific peptide or pharmaceutically acceptable salt thereof in the colon, and preferably releases a therapeutically effective amount of the MC1r-specific peptide or a pharmaceutically acceptable salt thereof in the colon.

In another aspect, the melanocortin receptor-specific peptide or a pharmaceutically acceptable salt thereof is Ac-Nle-cyclo(Glu-His-D-Phe-Arg-Dap)-Trp-NH₂ (SEQ ID NO:6) or a pharmaceutically acceptable salt thereof. In the formulation including Ac-Nle-cyclo(Glu-His-D-Phe-Arg-Dap)-Trp-NH₂ (SEQ ID NO:6) or a pharmaceutically acceptable salt thereof, the particle matrix, which may be a microparticle matrix, may include a delayed release polymer mixture comprising Eudragit® L100-55, Eudragit® S100 and Eudragit® FS30D present in a weight-to-weight ratio of L100-55 to S100 to FS30D selected from the group consisting of about 6:6:1, or about 6.2:6.2:1 or about 23.25:23:3.75. The Eudragit® L100-55, Eudragit® S100 and Eudragit® FS30D may be microparticles with a maximum particle size of no more than 1000 μm in diameter, preferably no more than about 600 μm in diameter, and with a minimum particle size of at least about 250 μm in diameter. The percentage of Ac-Nle-cyclo(Glu-His-D-Phe-Arg-Dap)-Trp-NH₂ (SEQ ID NO:6) or a pharmaceutically acceptable salt thereof of delayed release polymer may be no more than about 2% on a weight-to-weight basis, or alternatively no more than about 1% on a weight-to-weight basis, or alternatively no more than about 10% on a weight-to-weight basis. The formulation may further include at least one excipient selected from the group consisting of a surfactant, a disintegrant, a lubricant, and a binder.

In another aspect, the formulation comprising a melanocortin receptor-specific peptide or a pharmaceutically acceptable salt thereof disposed within a particle matrix comprising at least one delayed release polymer effects, when administered to a human patient, maximal release of the melanocortin receptor-specific peptide or a pharmaceutically acceptable salt thereof within the colon. In this aspect, the at least one delayed release polymer may be a pH-dependent release polymer, including a mixture comprising Eudragit® L100-55, Eudragit® S100 and Eudragit® FS30D present in a weight-to-weight ratio of L100-55 to S100 to FS30D selected from the group consisting of about 6:6:1, or about 6.2:6.2:1 or about 23.25:23:3.75.

In another aspect of the invention, in the formulation comprising a melanocortin receptor-specific peptide or a pharmaceutically acceptable salt thereof disposed within a particle matrix comprising at least one delayed release polymer, the melanocortin receptor-specific peptide or a pharmaceutically acceptable salt thereof is functionally active at the MC1r and at least one additional melanocortin receptor selected from the group consisting of the MC3r, the MC4r and the MC5r.

In another aspect, the invention provides a lower GI tract release pharmaceutical formulation prepared by a process comprising the steps of:

a. providing a solution admixture of Eudragit® L100-55, Eudragit® S100 and Eudragit® FS30D in a weight-to-weight ratio of L100-55 to S100 to FS30D selected from the group consisting of about 6:6:1, or about 6.2:6.2:1 or about 23.25:23:3.75;

b. adding Ac-Nle-cyclo(Glu-His-D-Phe-Arg-Dap)-Trp-NH₂ (SEQ ID NO:6) or a pharmaceutically acceptable salt thereof to the solution admixture;

c. drying the solution admixture comprising Ac-Nle-cyclo(Glu-His-D-Phe-Arg-Dap)-Trp-NH₂ (SEQ ID NO:6) or a pharmaceutically acceptable salt thereof; and

d. converting the dried admixture to microparticles wherein the resulting particle size is no more than about 1000 μm in diameter, and preferably wherein the resulting particle size is between about 250 μm and about 600 μm in diameter.

In the foregoing process, in one aspect no more than about 2% on a weight-to-weight basis of Ac-Nle-cyclo(Glu-His-D-Phe-Arg-Dap)-Trp-NH₂ (SEQ ID NO:6) or a pharmaceutically acceptable salt thereof is added to the solution admixture of Eudragit® L100-55, Eudragit® S100 and Eudragit® FS30D. In the process, drying may comprise vacuum drying. In the process, converting may comprise pulverizing the dried admixture and sieving through a screen.

In another aspect, the invention provides a modified-release formulation comprising

a MC1r-specific cyclic peptide or a pharmaceutically acceptable salt thereof as a single active pharmaceutical ingredient, and

at least one release controlling polymer selected from the group consisting of pH-dependent polymers and non-pH-dependent polymers;

wherein on oral administration to a human patient, the MC1r-specific cyclic peptide or a pharmaceutically acceptable salt thereof is delivered substantially intact to the lumen of the colon of the human patient.

In another aspect, the invention provides a pharmaceutical composition suitable for oral administration for treatment of an inflammatory bowel disease, the pharmaceutical composition comprising:

a tablet core, the tablet core comprising an active compound selected a MC1r-specific cyclic peptide or a pharmaceutically acceptable salt thereof as a single active pharmaceutical ingredient and a pharmaceutically acceptable excipient; and

an enteric coating.

In another aspect, the invention provides a pharmaceutical composition suitable for oral administration for treatment of an inflammatory bowel disease, the pharmaceutical composition comprising:

a melanocortin receptor-specific peptide or a pharmaceutically acceptable salt thereof disposed within an encapsulated microparticle matrix comprising at least one delayed release polymer; and

an enteric coating covering the capsule.

In the pharmaceutical composition the melanocortin receptor-specific peptide or a pharmaceutically acceptable salt thereof may be Ac-Nle-cyclo(Glu-His-D-Phe-Arg-Dap)-Trp-NH₂ (SEQ ID NO:6) or a pharmaceutically acceptable salt thereof, and the least one delayed release polymer may comprise pH-sensitive methyl methacrylate/methacrylic copolymers.

In yet another aspect, the invention provides a method of treating IBD in a human patient with IBD, comprising administering a melanocortin receptor-specific peptide or a pharmaceutically acceptable salt thereof disposed within a microparticle matrix comprising at least one delayed release polymer. In this method, the delayed release polymer may be a pH-dependent release polymer. The peptide or pharmaceutically acceptable salt thereof may be admixed within the microparticle matrix, thereby forming an admixture of the microparticle matrix and the peptide or pharmaceutically acceptable salt thereof. The admixture of the microparticle matrix and the peptide or pharmaceutically acceptable salt thereof may be disposed within an aqueous soluble capsule, including a gelatin capsule, which capsule may further comprise an enteric coating, including a pH-dependent release polymer. Alternatively, the admixture of the microparticle matrix and the peptide or pharmaceutically acceptable salt thereof may be formed into a tablet, and the tablet may further comprise an enteric coating, including a pH-dependent release polymer.

In the method of treating IBD in a human patient, the pH-dependent release polymer may comprise pH-sensitive methyl methacrylate/methacrylic copolymers, including copolymers selected from the group consisting of Eudragit® L100-55, Eudragit® S100 and Eudragit® FS30D. The Eudragit® L100-55, Eudragit® S100 and Eudragit® FS30D may be present in a weight-to-weight ratio of L100-55 to S100 to FS30D selected from the group consisting of about 6:6:1, or about 6.2:6.2:1 or about 23.25:23:3.75.

In the method of treating IBD in a patient, the melanocortin receptor-specific peptide or a pharmaceutically acceptable salt thereof may be a MC1r-specific peptide or a pharmaceutically acceptable salt thereof. The MC1r-specific peptide or a pharmaceutically acceptable salt thereof may have a functional EC₅₀ value at MC1r of less than about one nM. The MC1r-specific peptide or a pharmaceutically acceptable salt thereof may have a functional EC₅₀ value at the MC4r at least one hundred times less the functional EC₅₀ value at MC1r. In one aspect, in the method the MC1r-specific peptide or a pharmaceutically acceptable salt thereof has a functional EC₅₀ value at the MC4r of at least about 500 nM. In another aspect, the MC1r-specific peptide or a pharmaceutically acceptable salt thereof may be functionally inactive at the MC2r, the MC3r and the MC5r.

In one aspect of the method of treating IBD in a human patient with IBD, the melanocortin receptor-specific peptide or a pharmaceutically acceptable salt thereof may be Ac-Nle-cyclo(Glu-His-D-Phe-Arg-Dap)-Trp-NH₂ (SEQ ID NO:6) or a pharmaceutically acceptable salt thereof. In this aspect, the microparticle matrix may further be a mixture comprising Eudragit® L100-55, Eudragit® S100 and Eudragit® FS30D present in a weight-to-weight ratio of L100-55 to S100 to FS30D selected from the group consisting of about 6:6:1, or about 6.2:6.2:1 or about 23.25:23:3.75. The Eudragit® L100-55, Eudragit® S100 and Eudragit® FS30D may be microparticles with a maximum particle size of no more than 1000 μm in diameter, or alternatively no more than about 600 μm in diameter. The percentage of Ac-Nle-cyclo(Glu-His-D-Phe-Arg-Dap)-Trp-NH₂ (SEQ ID NO:6) or a pharmaceutically acceptable salt thereof of delayed release polymer is no more than about 2% on a weight-to-weight basis, or alternatively no more than about 1% on a weight-to-weight basis, or alternatively no more than about 10% on a weight-to-weight basis.

In one aspect of the method of treating IBD in a human patient with IBD, the at least one delayed release polymer effects, when administered to the human patient with IBD, maximal release of the melanocortin receptor-specific peptide or a pharmaceutically acceptable salt thereof within the colon. The at least one delayed release polymer may be a pH-dependent release polymer, optionally a mixture comprising Eudragit® L100-55, Eudragit® S100 and Eudragit® FS30D present in a weight-to-weight ratio of L100-55 to S100 to FS30D selected from the group consisting of about 6:6:1, or about 6.2:6.2:1 or about 23.25:23:3.75.

In another aspect of the method of treating IBD in a human patient with IBD, the melanocortin receptor-specific peptide or a pharmaceutically acceptable salt thereof is functionally active at the MC1r and at least one additional melanocortin receptor selected from the group consisting of the MC3r, the MC4r and the MC5r.

In another aspect, the present invention provides a melanocortin receptor-specific peptide-based pharmaceutical composition for use in treatment of GI tract melanocortin receptor-mediated diseases, indications, conditions and syndromes.

In another aspect, the present invention provides a peptide-based melanocortin receptor-specific pharmaceutical, wherein the peptide is a selective MC1r ligand disposed within a pH-dependent polymeric microparticle matrix, for use in treatment of MC1r associated IBD disorders, diseases, indications, conditions and/or syndromes.

In another aspect, the present invention provides a peptide melanocortin receptor-specific pharmaceutical for use in treatment wherein administration of the treatment is via oral administration of a polymeric matrix providing for release of the peptide within the GI tract, including the colon.

In another aspect, the present invention provides formulations and methods for employing specific MC1r cyclic peptides that may be employed for targeted delivery to the lumen of the lower GI tract, including the colon, utilizing a pH-dependent polymeric controlled release matrix.

In another aspect, the present invention provides formulations and methods for administration of specific MC1r cyclic peptides to receptors within the lumen of the lower GI tract wherein the peptides are delivered without any, or without any substantial, systemic delivery of such peptides, including without any substantial systemic delivery of such peptides to the cardiovascular circulation.

Yet another aspect of the present invention provides for site-specific delivery of a specific MC1r cyclic peptide to receptors within the lumen of the lower GI tract, including the colon, of a patient with IBD by means of oral administration of the peptide disposed within a pH-dependent polymeric microparticle matrix, wherein the peptide is delivered to and released within the lumen of the lower GI tract, including the colon, without any, or without any substantial, resulting presence of the peptide within the circulation of the patient.

Other aspects and novel features, and the further scope of applicability of the present invention will be set forth in part in the detailed description to follow, and in part will become apparent to those skilled in the art upon examination of the following, or may be learned by practice of the invention. The aspects of the invention may be realized and attained by means of the instrumentalities and combinations particularly pointed out in the appended claims.

### BRIEF DESCRIPTION OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more preferred embodiments of the invention and are not to be construed as limiting the invention.

FIGS. 1A and 1B are graphs of the effects of the peptide of Example 9.3 administered via colonic cannula and sulfasalazine administered orally on inflammation score (FIG. 1A) and colon weight (FIG. 1B) in rats with DNBS-induced bowel inflammation, where “*” indicates a p value of less than 0.05, IC is intracolonic, and PO is oral.

FIG. 2 is a graph of the progression of the peptide of Example 9.3 disposed within a microparticle matrix of Lot 41 administered via an oral capsule of the invention through the rat intestinal tract, wherein the “colon” comprises the rectum and distal colon, the “large intestine” comprises the distal intestine, and the “small intestine” comprises the proximal intestine.

FIGS. 3A and 3B are graphs of the effects of the peptide of Example 9.3 administered via an oral capsule of the invention and orally-administered sulfasalazine on baseline-corrected macroscopic damage scores (FIG. 3A) and baseline-corrected inflammation scores in rats with DNBS-induced bowel inflammation, where “*” indicates a p value of less than 0.05, “**” indicates a p value of less than 0.01, and “***” indicates a p value of less than 0.001.

FIG. 4 is a graph of dissolution of the peptide of Example 9.3 from Eudragit® microparticle Lots 23, 24 and 27 into phosphate buffer at pH 6.8.

FIG. 5 is a graph of dissolution of the peptide of Example 9.3 over time from various Eudragit® microparticles lots at pH ranges from pH 1.2 to pH 7.4.

FIG. 6 is a graph of dissolution of the peptide of Example 9.3 dissolution from Eudragit® microparticles Lots 23, 24, 27, and 31, at pH ranges from pH 1.2 to pH 7.4 over time with the peptide concentration at either 1% or 2%.

FIG. 7 is a graph of the dissolution profile of Lot 35, comprising 40% of Lot 29 (60% Eudragit® L-100-55/40% FS) and 60% of Lot 31R (Eudragit® S100), over time.

FIG. 8 is a graph of dissolution of the peptide of Example 9.3 from Lot 40 into buffer where buffer was pH adjusted over time, from pH 4.5-5.5 and pH 4.5-7.5.

FIG. 9 is a graph of cumulative release of the peptide of Example 9.3 over time and increasing pH (pH 4.5 to 7.5) for Lots 29, 34 and 38.

FIG. 10 is a graph of cumulative release of the peptide of Example 9.3 over time and increasing pH (pH 4.5 to 7.5) for Lots 38 and 41.

FIG. 11 is a graph of cumulative release of the peptide of Example 9.3 over time and increasing pH (pH 4.5 to 7.5) with two runs of Lot 41.

### DETAILED DESCRIPTION OF THE INVENTION

### 1.0 Definitions

Before proceeding with the description of the invention, certain terms are defined as set forth herein.

In the sequences given for the peptides according to the present invention, the amino acid residues have their conventional meaning as given in Chapter 2400 of the Manual of Patent Examining Procedure, 9^{th} Ed. Thus, “Nle” is norleucine, “Asp” is aspartic acid, “His” is histidine, “Phe” is phenylalanine, “Arg” is arginine, “Trp” is tryptophan, and “Lys” is lysine, and so on. It is to be understood that D-isomers are designated by a “D-” before the three-letter code or amino acid name, such that for example D-Phe is D-phenylalanine. Amino acid residues not encompassed by the foregoing include the following amino acids or amino acid side chains, it being understood that such amino acid residues may be L-isomers or D-isomers:

| | | Side Chain or |
|---|---|---|
| Abbreviation | Common Name | Amino Acid Structure |
| Cit | citrulline | 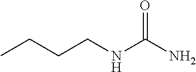 |
| Dab | diaminobutyric acid | 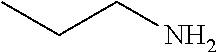 |
| Dap | diaminoproprionic acid | 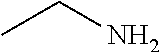 |
| hGlu | homoglutamic acid | 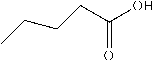 |
| Hyp | hydroxyproline | 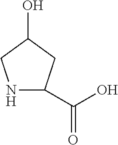 |
| Hyp(Bzl) | O-benzyl-hydroxyproline | 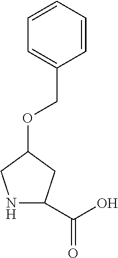 [SMILES: O=C(O)C1CC(OCc2ccccc2)CN1] |
| Nal 1 | 3-(1-naphthyl)alanine | 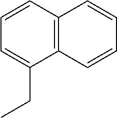 |
| Nal 2 | 3-(2-naphthyl)alanine | 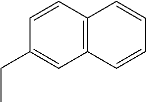 |
| Nle | norleucine | 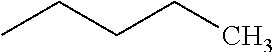 |
| Orn | ornithine | 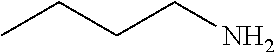 |
| Pro(4-Bzl) | 4-benzyl-proline | 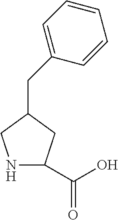 [SMILES: O=C(O)C1CC(Cc2ccccc2)CN1] |
| Pro(4-NH2) | 4-amino-proline | 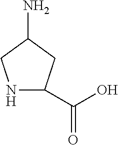 |
| Sar | sarcosine | 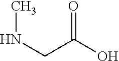 |

The term “alpha amino acid” includes any amino acid of the general structure

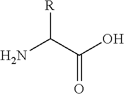

(depicted in its un-ionized form), where R is any side chain group or hydrogen, including without limitation the amino acid residues or side chain groups described in the preceding table and paragraph.

An “N-substituted amino acid” means any amino acid wherein an amino acid side chain moiety is covalently bonded to the backbone amino group, including optionally where there are no substituents other than H in the α-carbon position. Sarcosine is an example of an N-substituted amino acid. By way of example, sarcosine can be referred to as an N-substituted amino acid derivative of Ala, in that the amino acid side chain moiety of sarcosine and Ala is the same, methyl. Whenever a claim or description herein refers to an “amino acid”, such designation includes, but is not limited to, an ““N-substituted amino acid.”

The term “L- or D-isomer amino acid” or “L- or D-isomer amino acids” means any amino acid residue as defined herein, including specifically any alpha-amino acid, beta-amino acid, gamma-amino acid or delta-amino acid, including without limitation an amino acid that is directly coded by DNA, a post-translationally modified amino acid, an amino acid expressed by biological means other than directly by DNA, a proteinogenic or non-proteinogenic amino acid, or any synthetic or manmade amino acid.

Amino acids, including L- or D-isomer amino acids, are joined together by “amide bond” or amide linkages to form a covalent peptide bond linking a backbone carboxylic acid group of one amino acid with a backbone amino group of another amino acid, thereby forming a peptide bond (—C(═O)—NH—).

In certain instances groups may be substituted for an amino acid, such as particularly use of a dicarboxylic acid in place of an amino acid. One particular dicarboxylic acid utilized herein is succinic acid, abbreviated as “Suc”, which has the structural formula

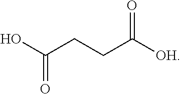

The term “alkane” includes linear or branched saturated hydrocarbons. Examples of linear alkane groups include methane, ethane, propane, and the like. Examples of branched or substituted alkane groups include methylbutane or dimethylbutane, methylpentane, dimethylpentane or trimethylpentane, and the like. In general, any alkyl group may be a substitutent of an alkane.

The term “alkene” includes unsaturated hydrocarbons that contain one or more double carbon-carbon bonds. Examples of such alkene groups include ethylene, propene, and the like.

The term “alkenyl” includes a linear monovalent hydrocarbon radical of two to six carbon atoms or a branched monovalent hydrocarbon radical of three to six carbon atoms containing at least one double bond; examples thereof include ethenyl, 2-propenyl, and the like.

The “alkyl” groups specified herein include those alkyl radicals of the designated length which are either straight or branched chain saturated aliphatic hydrocarbon groups. Non-liminiting examples of such alkyl radicals include methyl, ethyl, propyl, isopropyl, butyl, sec-butyl, tertiary butyl, pentyl, isopentyl, hexyl, isohexyl, and the like.

The term “alkyne” includes a linear monovalent hydrocarbon radical of two to six carbon atoms or a branched monovalent hydrocarbon radical of three to six carbon atoms containing at least one triple bond; examples thereof include ethyne, propyne, butyne, and the like.

The term “aryl” includes a monocyclic or bicyclic aromatic hydrocarbon radical of 6 to 12 ring atoms, and optionally substituted independently with one or more substituents selected from alkyl, haloalkyl, cycloalkyl, alkoxy, alkythio, halo, nitro, acyl, cyano, amino, monosubstituted amino, disubstituted amino, hydroxy, carboxy, or alkoxy-carbonyl. Examples of an aryl group include phenyl, biphenyl, naphthyl, 1-naphthyl, and 2-naphthyl, derivatives thereof, and the like.

The term “aralkyl” includes a radical —R^{a}R^{b} where R^{a} is an alkylene (a bivalent alkyl) group and R^{b} is an aryl group as defined above. Examples of aralkyl groups include benzyl, phenylethyl, 3-(3-chlorophenyl)-2-methylpentyl, and the like.

The term “aliphatic” includes compounds with hydrocarbon chains, such as for example alkanes, alkenes, alkynes, and derivatives thereof.

The term “acyl” includes a group R(C═O)—, where R is an organic group, such as an alkyl, aryl, heteroaryl, carbocyclyl or heterocyclyl. A non-limiting example is the acetyl group CHs—C(═O)—, referred to herein as “Ac”. As used herein, R may comprise a C₁ to C₁₇ linear or branched alkyl, cycloalkyl, alkylcycloalkyl, aryl or alkylaryl.

A peptide or aliphatic moiety is “acylated” when an alkyl or substituted alkyl group as defined above is bonded through one or more carbonyl {—(C═O)—} groups. A peptide is most usually acylated at the N-terminus.

The term “heteroaryl” includes mono- and bicyclic aromatic rings containing from 1 to 4 heteroatoms selected from nitrogen, oxygen and sulfur. 5- or 6-membered heteroaryl are monocyclic heteroaromatic rings; examples thereof include thiazole, oxazole, thiophene, furan, pyrrole, imidazole, isoxazole, pyrazole, triazole, thiadiazole, tetrazole, oxadiazole, pyridine, pyridazine, pyrimidine, pyrazine, and the like. Bicyclic heteroaromatic rings include, but are not limited to, benzothiadiazole, indole, benzothiophene, benzofuran, benzimidazole, benzisoxazole, benzothiazole, quinoline, benzotriazole, benzoxazole, isoquinoline, purine, furopyridine and thienopyridine.

As used herein, the term “amide” includes compounds that have a trivalent nitrogen attached to a carbonyl group, i.e. —C(═O)—NH₂ (i.e. primary amide), —C(═O)—NHR_{c} and —C(═O)—NR_{c}R_{d}, wherein each of R_{c} and R_{d} independently represents an organic group. When reference is made herein to a substituted amide group, it means that at least one of said organic groups (R_{c} and R_{d}) is substituted. Examples of amides include methylamide, ethylamide, propylamide, and the like.

An “imide” includes compounds containing an imido group (—C(═O)—NH—C(═O)—).

An “amine” includes compounds that contain an amino group (—NH₂), —NHRₐ and —NRₐR_{b}, wherein each of Rₐ and R_{b} independently represents an organic group. When reference is made herein to a substituted amine group, it means that at least one of the organic groups (Rₐ and R_{b}) is substituted.

A “nitrile” includes compounds that are carboxylic acid derivatives and contain a (—CN) group bound to an organic group.

The term “halogen” includes the halogen atoms fluorine, chlorine, bromine and iodine, and groups including one or more halogen atoms, such as —CF₃ and the like.

The term “composition”, as in pharmaceutical composition, encompasses a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions encompass any composition made by admixing an active ingredient and one or more pharmaceutically acceptable carriers.

By a melanocortin receptor “agonist” is meant an endogenous substance, drug substance or compound, including certain of the peptide compounds disclosed herein, which can interact with a melanocortin receptor and initiate a pharmacological response, including but not limited to activation of the receptor, including initiating signal transduction, such as adenyl cyclase activation, characteristic of the melanocortin receptor. A melanocortin receptor agonist may be an agonist at one or more of MC1r, MC2r, MC3r, MC4r and MC5r. For the present invention, a melanocortin receptor agonist which is an agonist at MC1r is preferred.

By “α-MSH” is meant the peptide Ac-Ser-Tyr-Ser-Met-Glu-His-Phe-Arg-Trp-Gly-Lys-Pro-Val-NH₂ (SEQ ID NO:2) and analogs and homologs thereof, including without limitation NDP-α-MSH.

By “NDP-α-MSH” is meant the peptide Ac-Ser-Tyr-Ser-Nle-Glu-His-D-Phe-Arg-Trp-Gly-Lys-Pro-Val-NH₂ (SEQ ID NO:3) and analogs and homologs thereof.

By “EC₅₀” is meant the molar concentration of an agonist, including a partial agonist, which produced 50% of the maximum possible response for that agonist. By way of example, a test compound which, at a concentration of 72 nM, produces 50% of the maximum possible response for that compound as determined in a cAMP assay in an MC1r cell expression system has an EC₅₀ of 72 nM. Unless otherwise specified, the molar concentration associated with an EC₅₀ determination is in nanomoles per liter (nM).

By “Ki (nM)” is meant the equilibrium inhibitor dissociation constant representing the molar concentration of a competing compound that binds to half the binding sites of a receptor at equilibrium in the absence of competitors. In general, the numeric value of the Ki is inversely correlated to the affinity of the compound for the receptor, such that if the Ki is low, the affinity is high. Ki may be determined using the equation of Cheng and Prusoff (Cheng Y., Prusoff W. H., Biochem. Pharmacol. 22: 3099-3108, 1973):

$$Ki = \frac{{EC}_{50}}{1 + \frac{[ ligand ]}{{K}_{D}}}$$

where “ligand” is the concentration of competitor and K_{D} is an inverse measure of receptor affinity for the competitor which produces 50% receptor occupancy by the competitor. Unless otherwise specified, the molar concentration associated with a Ki determination is in nM. Ki may be expressed in terms of specific receptors (e.g., MC1r, MC3r, MC4r or MC5r), specific species (e.g., human or murine), and specific ligands (e.g., α-MSH or NDP-α-MSH).

By “inhibition” is meant the percent attenuation, or decrease in receptor binding, in a competitive inhibition assay compared to a known standard. Thus, by “inhibition at 1 μM (NDP-α-MSH)” is meant the percent decrease in binding of NDP-α-MSH by addition of a determined amount of the compound to be tested, such as 1 μM of a test compound, such as under the assay conditions hereafter described. By way of example, a test compound that does not inhibit binding of NDP-α-MSH has a 0% inhibition, and a test compound that completely inhibits binding of NDP-α-MSH has a 100% inhibition. Typically, as described hereafter, a detectably labeled assay is used for competitive inhibition testing, such as with I¹²⁵-labeled NDP-α-MSH, or a lanthanide chelate fluorescent assay, such as with Eu-NDP-α-MSH. However, other methods of testing competitive inhibition are known, including use of different label or tag systems, and in general any method known in the art for testing competitive inhibition may be employed in this invention. It may thus be seen that “inhibition” is one measure to determine whether a test compound attenuates binding of α-MSH to melanocortin receptors.

By “binding affinity” is meant the ability of a compound or drug to bind to its biological target, expressed herein as Ki (nM).

By “Eₘₐₓ” is meant the maximal functional activity achievable by a compound in a specified melanocortin receptor expressing cell system, such as the maximal stimulation of adenylyl cyclase. The maximal stimulation achieved by NDP-α-MSH is designated as an Eₘₐₓ of 100% and a compound capable of stimulating half the maximal activity of NDP-α-MSH is designated as having an Eₘₐₓ of 50%. A compound of this invention that under assay conditions described herein has an Eₘₐₓ of 70% or higher may be classified as an agonist, a compound with an Eₘₐₓ between 10% and 70% may be classified as a partial agonist, and a compound with an Eₘₐₓ below 10% may be classified as inactive.

In general, “functional activity” is a measure of the signaling of a receptor, or measure of a change in receptor-associated signaling, such as with a melanocortin receptor, upon activation of the receptor by a compound. Melanocortin receptors initiate signal transduction through activation of heterotrimeric G proteins. In one aspect, melanocortin receptors signal through Gas, which catalyzes production of cAMP by adenylyl cyclase. Thus, determination of stimulation of adenylyl cyclase, such as determination of maximal stimulation of adenylyl cyclase, is one measure of functional activity, and is a primary measure exemplified herein. However, it is to be understood that alternative measures of functional activity may be employed in the practice of this invention, and are specifically contemplated and included within the scope of this invention. Thus, in one example intracellular free calcium may be measured using specific fluorescent molecules binding to calcium, such as Fura2, reported by and using the methods disclosed in Mountjoy K. G. et al., Melanocortin receptor-medicated mobilization of intracellular free calcium in HEK293 cells. Physiol Genomics 5:11-19, 2001, or Newman et al., Activation of the melanocortin-4 receptor mobilizes intracellular free calcium in immortalized hypothalamic neurons. J Surg Res:132:201-207, 2006. Fluo-4 is an alternative calcium binding dye that is also commonly used (Nohr et al., The orphan G protein-coupled receptor GPR139 is activated by the peptides: Adrenocorticotropic hormone (ACTH), α-, and β-melanocyte stimulating hormone (α-MSH, and β-MSH), and the conserved core motif HFRW. Neurochem Int 102: 105-113, 2017). Further upstream to the Ca2⁺ release event and in the same pathway, it is also possible to measure activation by measurement of the production of inositol triphosphate or diacylglycerol from phosphatidylinositol 4,5-biphosphate, such as the commercially-available HTRF assays (Liu et al., Comparison on functional assays for Gq-coupled GPCRs by measuring inositol monophospate-1 and intracellular calcium in 1536-well plate format. Curr Chem Genomics 1: 70-77, 2008). Yet another measure of functional activity is receptor internalization, resulting from activation of regulatory pathways, such as using the methods disclosed in Nickolls S. A. et al., Functional selectivity of melanocortin 4 receptor peptide and nonpeptide agonists: evidence for ligand specific conformational states. J Pharm Exper Therapeutics 313:1281-1288, 2005. Yet another measure of functional activity is the exchange, and exchange rate, of nucleotides associated with activation of a G protein receptor, such as the exchange of GDP (guanosine diphosphate) for GTP (guanosine triphosphase) on the G protein α subunit, which may be measured by any number of means, including a radioassay using guanosine 5′-(γ-[³⁵S]thio)-triphosphate, as disclosed in Manning D. R., Measures of efficacy using G proteins as endpoints: differential engagement of G proteins through single receptors. Mol Pharmacol 62:451-452, 2002. A relatively new assay platform has been devised to measure the activity/engagement of the 14 different Gα species belonging to the Gi, Gq, Gs, Gi2/i3 subfamilies as it relates to the receptor using BRET (bioluminescence resonance energy transfer)-based biosensors to measure the disengagement of the Gα and Gγ subunits upon ligand binding (Zhao et al., Biased signaling of protease-activated receptors. Front Endocrinol 5:67, 2014, van der Westhuizen et al., Quantification of ligand bias for clinically relevant β2-adrenergic receptor ligands: Implications for drug taxonomy. Molecular Pharm 85:492-509, 2014). Various gene-based assays have been developed for measuring activation of G-coupled proteins, such as those disclosed in Chen W. et al., A colorimetric assay from measuring activation of Gs- and Gq-coupled signaling pathways. Anal Biochem 226:349-354, 1995; Kent T. C. et al., Development of a generic dual-reporter gene assay for screening G-protein-coupled receptors. Biomol Screening, 5:437-446, 2005; or Kotarsky K. et al., Improved receptor gene assays used to identify ligands acting on orphan seven-transmembrane receptors. Pharmacology & Toxicology 93:249-258, 2003. The colorimetric assay of Chen et al. has been adapted for use in measuring melanocortin receptor activation, as disclosed in Hruby V. J. et al., Cyclic lactam α-melanocortin analogues of Ac-Nle⁴-cyclo[Asp⁵,D-Phe⁷,Lys¹⁰] α-melanocyte-stimulating hormone-(4-10)-NH₂ with bulky aromatic amino acids at position 7 shows high antagonist potency and selectivity at specific melanocortin receptors. J Med Chem 38:3454-3461, 1995. In general, functional activity may be measured by any method, including methods of determining activation and/or signaling of a G-coupled receptor, and further including methods which may be hereafter developed or reported. Each of the foregoing articles, and the methods disclosed therein, is incorporated here by reference as if set forth in full.

A peptide is “functionally inactive” when the EC₅₀ value for such peptide, if ascertainable, is greater than about 1,000 nM.

The abbreviation “μm” is the symbol of an SI unit of measure known as a micrometer or micrometre, and also commonly known as a micron.

The term “particle,” as used herein, includes, without any limitations on the nature and size thereof, any particles, microparticles, spheres, beads, granules, pellets, particulates or any structural units that may be incorporated into an oral dosage form, and includes a “microparticle,” which as used herein includes a particle with a diameter of less than about 1000 μm.

The terms “treat,” “treating” and “treatment,” as used herein, contemplate an action that occurs while a patient is suffering from the specified disease or disorder, which reduces the severity of the disease or disorder.

As used herein, the term “pharmacologically effective amount” (including “therapeutically effective amount”) means an amount of a peptide administered according to the invention that is sufficient to induce a desired therapeutic or biological effect.

As used herein, the term “prophylactically effective” or “preventive” means the amount of a compound including a peptide of the invention that will prevent or inhibit affliction or mitigate affliction of a mammal with a medical condition that a medical doctor or other clinician is trying to prevent, inhibit, or mitigate before a patient begins to suffer from the specified disease or disorder.

### 2.0 Formulations and Uses

2.1 In a preferred embodiment, the melanocortin receptor-specific peptide, preferably a MC1r-specific cyclic peptide or a pharmaceutically acceptable salt thereof, is formulated in a pH dependent release form wherein the melanocortin receptor-specific peptide or a pharmaceutically acceptable salt thereof disposed within a particle or microparticle matrix comprising at least one delayed release polymer. The peptide or pharmaceutically acceptable salt thereof may be admixed within the microparticle matrix, thereby forming an admixture of the microparticle matrix and the peptide or pharmaceutically acceptable salt thereof. The admixture of the microparticle matrix and the peptide or pharmaceutically acceptable salt thereof may be disposed within an aqueous soluble capsule, which may be a gelatin capsule. Alternatively, the admixture of the microparticle matrix and the peptide or pharmaceutically acceptable salt thereof may be formed into a tablet, and the tablet may further comprise at least one of a seal coating and an enteric coating. The at least one delayed release polymer may include a pH-dependent release polymer, optionally comprising pH-sensitive methyl methacrylate/methacrylic copolymers, such as copolymers selected from the group consisting of Eudragit® L100-55, Eudragit® S100 and Eudragit® FS30D. The Eudragit® L100-55, Eudragit® S100 and Eudragit® FS30D copolymers may be present in a weight-to-weight ratio of L100-55 to S100 to FS30D selected from the group consisting of about 6:6:1, or about 6.2:6.2:1 or about 23.25:23:3.75.

In another embodiment, the melanocortin receptor-specific peptide or a pharmaceutically acceptable salt thereof is Ac-Nle-cyclo(Glu-His-D-Phe-Arg-Dap)-Trp-NH₂ (SEQ ID NO:6) or a pharmaceutically acceptable salt thereof. In the formulation including Ac-Nle-cyclo(Glu-His-D-Phe-Arg-Dap)-Trp-NH₂ (SEQ ID NO:6) or a pharmaceutically acceptable salt thereof, the particle or microparticle matrix may include a pH dependent delayed release polymer mixture comprising Eudragit® L100-55, Eudragit® S100 and Eudragit® FS30D present in a weight-to-weight ratio of L100-55 to S100 to FS30D selected from the group consisting of about 6:6:1, or about 6.2:6.2:1 or about 23.25:23:3.75. The Eudragit® L100-55, Eudragit® S100 and Eudragit® FS30D may be particles, such as microparticles, with a maximum particle size of no more than 1000 μm in diameter, preferably no more than about 600 μm in diameter, and further preferably at least about 250 μm in diameter. In one aspect, the maximum particle size may comprise at least about 1500 μm in diameter, 1400 μm in diameter, 1300 μm in diameter, 1200 μm in diameter, 1100 μm in diameter, 1000 μm in diameter, 900 μm in diameter, 800 μm in diameter, 700 μm in diameter, 600 μm in diameter, or 500 μm in diameter. In another aspect, the minimum particle may be no less than about 2.5 μm in diameter, 5 μm in diameter, 10 μm in diameter, 15 μm in diameter, 20 μm in diameter, 25 μm in diameter, 50 μm in diameter, 75 μm in diameter, 100 μm in diameter, 125 μm in diameter, 150 μm in diameter, 175 μm in diameter, 200 μm in diameter, 225 μm in diameter, 250 μm in diameter, 300 μm in diameter, 350 μm in diameter, or 400 μm in diameter. In yet another aspect, the minimum and maximum diameters are selected from the foregoing groups, and the difference between the minimum particle diameter and the maximum particle diameter is no more than about 100 μm, 125 μm, 150 μm, 200 μm, 250 μm, 300 μm, 350 μm, 400 μm, 450 μm, 500 μm, 550 μm, or 600 μm. In part, the maximum particle diameter, the minimum particle diameter and the difference between the minimum and maximum particle diameters can be optimized to obtain maximal delivery of the melanocortin receptor-specific peptide to the region of the GI tract desired to be treated.

In some embodiments, the melanocortin receptor-specific peptide, preferably a MC1r-specific cyclic peptide or a pharmaceutically acceptable salt thereof, is formulated in a pH dependent release form. Alternatively, such peptides are formulated in a form that releases the peptides at a specific region of the GI tract, such as the duodenum, jejunum, ileum, terminal ileum, ascending colon, traverse colon, descending colon, sigmoid colon or rectum. In one aspect, the formulation may contain an inert carrier coated with the MC1r-specific cyclic peptide or a pharmaceutically acceptable salt thereof and an enteric coating which releases the peptide at a specific pH (such as pH 5 or pH 7). In one aspect, a preferred pH for duodenum or jejunum release is pH 4.5-5.5 or pH 5.5-6.5. In another aspect, a preferred pH for ileum, terminal ileum, or colon release is pH 5.5-6.5 or pH 6.5-7.5. If an inert carrier is utilized, it may include, but is not limited to, mannitol, lactose, a microcrystalline cellulose, or starch.

For certain embodiments and IBD indications such as UC, it is desirable to utilize an oral compositive that commences release of the active drug, such as MC1r-specific cyclic peptide or a pharmaceutically acceptable salt thereof, at a pH of about 5.5, but releases no more than less than 20% of the active drug at pH 5.5, and releases not less than 80% of the active drug at a pH greater than about 6.0, or alternatively about 6.5, over a period more than two hours but less than seven hours, preferably over a period of about four to about seven hours.

In another embodiment, the MC1r-specific cyclic peptide or a pharmaceutically acceptable salt thereof is formulated in a particle or microparticle matrix, such as a delayed release polymer mixture or a pH dependent release polymer mixture, disposed within a capsule, which capsule may further include a seal coating or an enteric coating, or both. The pH dependent release polymer may include a polymer mixture comprising Eudragit® L100-55, Eudragit® S100 and Eudragit® FS30D present in a weight-to-weight ratio of L100-55 to S100 to FS30D selected from the group consisting of about 6:6:1, or about 6.2:6.2:1 or about 23.25:23:3.75. The Eudragit® L100-55, Eudragit® S100 and Eudragit® FS30D may be microparticles with a maximum particle size of no more than 1000 μm in diameter, preferably no more than about 600 μm in diameter, and further preferably at least about 25 μm in diameter, or at least about 250 μm in diameter.

In another embodiment, the MC1r-specific cyclic peptide or a pharmaceutically acceptable salt thereof is formulated in particulate or tablet form that includes a tablet core, a seal coating, and an enteric coating, where the tablet core includes one or more pharmaceutically acceptable excipients and the MC1r-specific cyclic peptide or a pharmaceutically acceptable salt thereof. By way of example and not limitation, the formulation of the tablet core may include a sugar alcohol, such as arabitol, erythritol, glycerol, isomalt, lactitol, maltitol, mannitol, sorbitol, or xylitol, or a microcrystalline cellulose with any desired average particle size, such as about 50 μm, about 100 μm, about 250 μm or any desire average particle size preferably less than about 1,000 μm. The tablet or other formulation may further include pharmaceutically acceptable excipients such as povidone, sodium lauryl sulphate, sodium starch glycollate, a salt of citrate such as sodium citrate or magnesium stearate. Such excipients comprise agents that may serve as a surfactant, a disintegrant, a lubricant, or a binder. Common pharmaceutical binders such as povidone, diluents, glidants, fillers such as microcrystalline cellulose, lubricants such as magnesium stearate, disintegrants such as croscarmellose sodium, preservatives, colorants and the like may thus be employed.

2.2 The compositions, formulations and methods disclosed herein can be used for both medical applications and animal husbandry or veterinary applications. Typically, the methods are used in humans, but may also be used in other mammals. The term “patient” denotes a mammalian individual, and is so used throughout the specification and in the claims. The primary applications of the present invention involve human patients, but the present invention may be applied to laboratory, farm, zoo, wildlife, pet, sport or other animals. Clinical indications and specific utilities include the following:

Peptides, compositions, formulations and methods of the present invention are directed towards the treatment of IBD, including but not limited to UC and Crohn's disease, in a subject. In another aspect, the inflammatory disease includes a form of IBD, such as Crohn's disease, UC, collagenous colitis, lymphocytic colitis, ischemic colitis, diversion colitis, Behçet's syndrome, infective colitis and indeterminate colitis.

Expression of various cytokines is increased during an inflammatory process, including an inflammatory process secondary to or accompanying certain forms of IBD. TNF-α is a pleiotropic cytokine produced mainly by macrophages, and also by other types of cells. Other cytokines which increase during an inflammatory process include IL-1 and IL-6. While cytokines such as TNF-α have beneficial effects in many instances, significantly increased levels, or increased levels for a substantial period of time, can have pathological effects.

In one embodiment, the invention is directed to methods of using one or more of the peptides of the present invention to decrease pro-inflammatory cytokine production and expression, including decreasing pro-inflammatory cytokine production and expression secondary to IBD. The decrease in pro-inflammatory cytokine production and expression, including without limitation one or more of TNF-α, IL-1 and IL-6, occurs preferably within a short time period following release of a peptide from a composition at the site of disease, such as IBD.

In a related embodiment, the invention is directed to methods of using one or more of the peptides of the present invention to increase anti-inflammatory cytokine production and expression. The increase in anti-inflammatory cytokine production and expression, including without limitation IL-10, occurs preferably within a short time period following release of a peptide from a composition at the site of disease, such as IBD.

In general, the actual quantity of MC1r-specific cyclic peptide or a pharmaceutically acceptable salt thereof administered to a patient will vary between fairly wide ranges depending upon the mode of administration, the formulation used, and the response desired. The dosage for treatment is administration, by any of the foregoing means or any other means known in the art, of an amount sufficient to bring about the desired therapeutic effect. Thus, a therapeutically effective amount includes an amount of a peptide or pharmaceutical composition of the present invention that is sufficient to therapeutically alleviate IBD in a patient, or to prevent or delay onset or recurrence of IBD, including UC and Crohn's disease, or to be prophylactically effective or preventive in preventing or limiting recurrences of exacerbations of IBD, including UC and Crohn's disease.

In general, the MC1r-specific cyclic peptide or a pharmaceutically acceptable salt thereof utilized in the practice of the invention are highly active. For example, the cyclic peptide can be administered to the lumen of the GI tract, such as the lumen of the colon or large intestinal, preferably proximal the site of IBD or other disease, at about 0.01, 0.05, 0.1, 0.5, 1, 5, 50, 100, 500, 1000 or 5000 μg/kg body weight, depending on the specific peptide selected, the delivery formulation, the desired therapeutic response, and other factors known to those of skill in the art.

### 3.0 Combination Therapy for Certain Indications

The peptides, compositions and methods of the present invention may be used for treatment of IBD, UC or Crohn's disease, or any disease, indication, condition or syndrome of the GI tract which is MC1r mediated or responsive, by administration in combination with one or more other pharmaceutically active compounds. Such combination administration may be by means of a single dosage form which includes both a peptide of the present invention and one more other pharmaceutically active compounds, such single dosage form including a tablet or capsule. Alternatively, combination administration may be by means of administration of two different dosage forms, with one dosage form containing a peptide of the present invention, and the other dosage form including another pharmaceutically active compound. In this instance, the dosage forms may be the same or different. The term “coadminister” indicates that each of at least two compounds in the combination therapy are administered during a time frame wherein the respective periods of biological activity or effects overlap. Thus the term includes sequential as well as concurrent administration of compounds where one compound is one or more of the peptides of the present invention. If more than one compound is coadministered, the routes of administration of the two or more compounds need not be the same. Without meaning to limit combination therapies, the following exemplifies certain combination therapies which may be employed.

For the treatment of inflammation-related diseases, indications, conditions and syndromes of the GI tract, peptides of the present invention may be used in combination therapy, including by means of coadministration, with one or more anti-inflammatory agents. One class of anti-inflammatory agent is glucocorticoids, including but not limited to cortisone, including cortisone acetate, hydrocortisone, prednisone, prednisolone, methylprednisolone, dexamethasone, betamethasone, triamcinolone, beclometasone, prednisone, fludrocortisone acetate, deoxycorticosterone acetate and aldosterone. Another class of anti-inflammatory agent is aminosalicylates, including but not limited to 5-aminosalicyclic acid, such as mesalamine, balsalazide and olsalazine.

Other anti-inflammatory agents that may be used in combination therapy, including by means of coadministration, include aspirin, non-steroidal antiinflammatory drugs (NSAIDs) (such as ibuprofen and naproxen), TNF-α inhibitors (such as tenidap and rapamycin or derivatives thereof), or TNF-α antagonists (e.g., infliximab, OR1384), cyclooxygenase inhibitors (i.e., COX-1 and/or COX-2 inhibitors), CTLA4-Ig agonists/antagonists, CD40 ligand antagonists, IMPDH inhibitors, such as mycophenolate, integrin antagonists, alpha-4 beta-7 integrin antagonists, cell adhesion inhibitors, interferon gamma antagonists, ICAM-1, prostaglandin synthesis inhibitors, budesonide, clofazimine, p38 mitogen-activated protein kinase inhibitors, protein tyrosine kinase (PTK) inhibitors, IKK inhibitors, other therapies for the treatment of irritable bowel syndrome (e.g., such as those disclosed in U.S. Pat. No. 6,184,231), or other NF-κB inhibitors, such as corticosteroids, calphostin, CSAIDs, 4-substituted imidazo[1,2-A]quinoxalines as disclosed in U.S. Pat. No. 4,200,750; Interleukin-10, salicylates, nitric oxide, and other immunosuppressants; and nuclear translocation inhibitors, such as deoxyspergualin Immunosuppressant drugs that may be coadministered include azathioprine, mercaptopurine, cyclosporine and methotrexate. Coadministration can also be employed with tumor necrosis factor (TNF)-alpha inhibitors such as infliximab, adalimumab and golimumab. Other biologic therapies that may be used include natalizumab, vedolizumab and ustekinumab. Co-administration may also be employed with proton pump inhibitors (such as omeprazole, pantoprazole, esomeprazole, lansoprazole, rabeprazole, dexlansoprazole, rabeprazole sodium, omeprazole magnesium, pantoprazole sodium, naproxen/esomeprazole, esomeprazole magnesium, esomeprazole sodium or omeprazole/vicarbonate ion), or with antibiotics to control small intestinal bacterial overgrowth (such as rifaximin or neomycin).

### 4.0 Methods of Making Multi-Particulate Delivery Formulations

In one aspect, peptides employed in the present invention, including MC1r-specific peptides, are formulated for oral delivery of intact peptide to the lumen of the GI tract, preferably the lumen of lower regions the GI tract, and further preferably prior to, including immediately prior to, any situs of disease, such as IBD, in the GI tract. Bypassing the stomach and upper regions of the GI tract, such as the small intestine, to deliver drugs to the lower regions of the GI tract is desired for many drug molecules, particularly proteinaceous drugs comprising proteins or peptides. The mouth and stomach include various enzymes which can break amino acid chains. The small intestine produces a variety of peptidases which can reduce amino acid chains, including peptides, to small units, including dipeptides and single amino acid residues, which can be absorbed and digested. Thus, for delivery of intact peptides to the lumen of the lower GI tract, including the colon, a method and formulation must be employed that transits the stomach and upper regions of the GI tract without peptidic degradation. This approach may also be used if the peptide is not stable in the acidic milieu of the stomach due to pH or enzymatic activity.

There are several approaches which may conceptually be utilized to achieve lower GI tract targeting, including the use of prodrugs, coating with pH-sensitive polymers, design of time-release dosage forms, or utilization of biodegradable polymers such as azopolymers and polysaccharides that degrade exclusively by the colonic bacteria. Each system has advantages as well as disadvantages.

Single-unit dosage forms for colonic delivery may suffer from the disadvantage of premature disintegration of the formulation due to high inter- and intra-subject viability and poor reproducibility which may lead to loss of local therapeutic action in the colon. Multi-particulate delivery systems offer advantages such as better bioavailability, decreased risk of local irritation and predictable gastric emptying.

Thus in one aspect the invention provides particulate dosage forms containing a melanocortin receptor-specific peptide, such as a MC1r-specific cyclic peptide or a pharmaceutically acceptable salt thereof, which particulate form protects the melanocortin receptor-specific peptide or pharmaceutically acceptable salt thereof while in the acid environment of the stomach, and prevents or limits protease degradation in the small intestine or upper GI tract, but releases the intact melanocortin receptor-specific peptide or pharmaceutically acceptable salt thereof in the lower GI tract, such as the large intestine or colon. By this means, the melanocortin receptor-specific peptide or pharmaceutically acceptable salt thereof binds to and agonizes one or more MC receptors, preferably MC1r, present on or in the lumen of the lower GI tract, including the large intestine or colon, or proximal to the lumen of the lower GI tract, including the large intestine or colon, thereby effecting a therapeutic response.

This approach may be employed by utilizing delayed release (enteric) microparticles using pH-sensitive methyl methacrylate/methacrylic copolymers. One form of pH-sensitive methyl methacrylate/methacrylic copolymers that may be utilized are Eudragit® polymers manufactured by Evonik Industries, it being understood that the use of other and different pH-sensitive methyl methacrylate/methacrylic copolymers, and other and different pH-sensitive polymers or copolymers, may be employed in the invention.

The melanocortin receptor-specific peptide may constitute from about 0.1% to about 30%, on a weight-to-weight basis, of the pH-sensitive delayed release particles. Preferable the melanocortin receptor-specific peptide constitutes about 1% to about 10%, or about 2% to about 5%, on a weight-to-weight basis, of the pH-sensitive delayed release particles.

The particles or microparticles may be filled into capsules, such as hard gelatin capsules, or may be formulated into tablets, beads, granules, powders, caplets, troches, sachets, cachets, pouches, gums, sprinkles, and suspensions or the like. In one aspect, if the particles comprising a melanocortin receptor-specific peptide, preferably a MC1r-specific cyclic peptide or a pharmaceutically acceptable salt thereof, and pH-sensitive methyl methacrylate/methacrylic copolymers are formulated into a solid form such as a capsule or a tablet, the capsule or tablet may be coated with a seal coating or an enteric coating, or both. In general, any solid forms of drug delivery, including tablets, bead, granules, caplets or the like, may be coated with a seal coating or an enteric coating, or both. The enteric coatings may comprise pH-sensitive sensitive methyl methacrylate/methacrylic copolymers.

In one aspect, the invention provides a formulation, dosage form and method wherein less than 10% of the active drug, such as a melanocortin receptor-specific peptide, MC1r-specific cyclic peptide or a pharmaceutically acceptable salt thereof, is released in an acid pH from about 1 to about 3 in a period of two hours, less than an additional 10% of the active drug is released in an acid pH from about 4.5 to 5.5 in a period of one hour, and not less than 80% of the active drug is released at a pH of greater than about 6 in a period of four to seven hours.

Of particular utility in the invention are pH-dependent polymethacrylates such as Eudragit® L100-55, Eudragit® L100, Eudragit® S100 and Eudragit® FS30D. These polymethacrylates comprise:

Eudragit® L100-55: Solid substance. The product contains 0.7% Sodium Laurilsulfate Ph. Eur./NF and 2.3% Polysorbate 80 Ph. Eur./NF on solid substance. Eudragit® L100-55 contains an anionic copolymer based on methacrylic acid and ethyl acrylate. The ratio of the free carboxyl groups to the ester groups is approximately 1:1. The monomers are randomly distributed along the copolymer chain. Based on SEC method the weight average molar mass (Mw) of Eudragit® L100-55 is approximately 320,000 g/mol.

Eudragit® L100: Solid substance. The product contains 0.3% Sodium Laurylsulfate Ph. Eur./NF on solid substance. Eudragit® L100 is an anionic copolymer based on methacrylic acid and methyl methacrylate. The ratio of the free carboxyl groups to the ester groups is approximately 1:1 in Eudragit® L100. Based on SEC method the weight average molar mass (Mw) of Eudragit® L100 is approximately 125,000 g/mol.

Eudragit® S100: Solid substance. The product contains 0.3% Sodium Laurylsulfate Ph. Eur./NF on solid substance. Eudragit® S100 is an anionic copolymer based on methacrylic acid and methyl methacrylate. The ratio of the free carboxyl groups to the ester groups is approximately 1:2 in Eudragit® S100. Based on SEC method the weight average molar mass (Mw) of Eudragit® S100 is approximately 125,000 g/mol.

Eudragit® FS30D: Supplied as an aqueous dispersion with 30% dry substance. The water is tested according to the specifications of “Purified Water in bulk” Ph. Eur. and according to the specifications for Conductivity of “Purified Water” USP. The dispersion contains 0.3% Sodium Laurilsulfate Ph. Eur./NF and 1.2% Polysorbate 80 Ph. Eur./NF on solid substance, as emulsifiers. Eudragit® FS30D is the aqueous dispersion of an anionic copolymer based on methyl acrylate, methyl methacrylate and methacrylic acid. The ratio of the free carboxyl groups to the ester groups is approximately 1:10. The monomers are randomly distributed along the copolymer chain. Based on SEC method the weight average molar mass (Mw) of Eudragit® FS30D is approximately 280,000 g/mol.

1 g of Eudragit® L100, Eudragit® L100-55 or Eudragit® S100 dissolves in 7 g methanol, ethanol, in aqueous isopropyl alcohol and in acetone (containing approximately 3% water), as well as in 1 N sodium hydroxide, to give clear to cloudy solutions. These specific Eudragit® preparations are practically insoluble in ethyl acetate, methylene chloride, petroleum ether and water. Eudragit® L100-55 dissolves above pH 5.5; Eudragit® L100 dissolves above pH 6.0; Eudragit® S100 dissolves above pH 7.0 and Eudragit® FS30D dissolves above pH 7.0.

Various techniques are available for drug encapsulation. In one aspect, microparticle formation through solid dispersion followed by micronization may be utilized, which is simple and provides for high encapsulation efficiency and high yield.

To make the drug product, the melanocortin receptor-specific peptide, MC1r-specific cyclic peptide or pharmaceutically acceptable salt thereof may be dispersed in a suitable solvent such as acetone, methanol or water, or combinations of some or all of the foregoing. The Eudragit® copolymer or copolymers may be dissolved in methanol or acetone. The drug dispersion comprising the peptide is added to the copolymer solution with stirring. The resulting mixture is then vacuum dried, pulverized and sieved through a suitable screen. In one aspect, 30-mesh over 60-mesh screens are employed wherein the resulting particle size collected on the 60-mesh screen is between 250 to 600 μm in diameter. In another aspect, the particles collected on the 60-mesh screen are suspended or rinsed with 0.1 M hydrochloric acid solution pH 1.2, to remove surface MC1r peptide drug molecules, and subsequently dried. The resulting microparticles may be encapsulated or tableted. The filled capsules or tablets may also be enteric coated to further reduce the amount of drug released in the upper gastrointestinal tract, thereby allowing more drug to reach the colon.

Alternatively, methanol, methanol-water (such as a 2:1 mixture) and water may be employed as a solvent/dispersant for melanocortin receptor-specific peptide or pharmaceutically acceptable salts thereof. Alternative, acetone or acetone-water may be employed as a solvent/dispersant. In one aspect, if water is employed it may be used in such quantity as is not greater than about 3% of the amount of acetone used in dissolving the copolymer(s).

The formulations employed in the invention may, in one embodiment, incorporates Eudragit® polymers, such as for example L100-55, that are soluble and release an associated peptide at a lower pH, combined with polymers that are soluble and release an associated peptide at a higher pH, such as Eudragit® S100 or FS30D, or both. This blend assures release across a wider pH range. The wider pH range release is superior to prior art formulations for colon release at a single specific pH, because it allows partial release higher up in the GI tract where disease may be present in some patients, and also because it provides for release in a portion of the GI tract of patients that has a lower pH GI tract than seen in normal subjects, with the lower pH value being due to the IBD disease state. If desired, as in the case of lower GI tract pH in certain IBD disease states, different Eudragit® polymers (for example, Eudragit® L100-55) may be partially neutralized and/or other additives such as alginic, sorbic or succinic acid or their salts added, to increase the release of the drug at a lower pH, such as 4.5 to 5.5. The utilization of a wide range pH release profile combined with a melanocortin receptor-specific peptide, which melanocortin receptor-specific peptide binds to receptors present on or in the luminal surface of the GI tract rather than providing a therapeutic benefit through systemic absorption, provides a therapeutic agent suitable for treatment of a wide variety of patients. Thus, because there is little or no systemic absorption of the melanocortin receptor-specific peptide, and little or no therapeutic benefit from any systemic absorption that may occur, the formulation is preferably intended to provide benefit through the range of the GI tract in which disease is or may be present, and to provide sufficient dosing within such range as to effect a remission or cure of the IBD. It is particularly important to note that because there is little or no systemic absorption of the melanocortin receptor-specific peptide, there is little or systemic toxicity or systemic side effects or adverse effects that limit the quantity of melanocortin receptor-specific peptide that may be delivered to the lumen of the gastrointestinal tract.

In some embodiments, combinations of different pH-sensitive methyl methacrylate/methacrylic copolymers formulated as delayed release (enteric) particles or microparticles are employed. In some embodiments, the particles or microparticles comprise Eudragit® L100-55 and Eudragit® S100 in a weight-to-weight ratio of L100-55 to S-100 of about 1:1, or about 2:3, or about 1:2, or about 3:2, or about 2:1. In other embodiments, the particles or microparticles comprise Eudragit® L100-55, Eudragit® L100 and Eudragit® S100 in a weight-to-weight ratio of L100-55 to L100 to S-100 of about 1:1:1, or about 4:3:3, or about 3:4:3, or about 1:1:1, or about 1:2:1, or about 1:2:2, or about 2:1:1, or about 2:2:1, or about 2:1:2. In other embodiments, the particles or microparticles comprise Eudragit® L100-55, Eudragit® S100 and Eudragit® FS30D in a weight-to-weight ratio of L100-55 to S100 to FS30D of about 6:6:1, or about 23.35:23:3.75, or about 5:5:1, or about 4:4:1, or about 6:5:1, or about 5:6:1, or about 3:3:1, or about 6:5:2, or about 5:6:2. Particularly preferred is a weight-to-weight ratio of L100-55 to S100 to FS30D of about 6:6:1 or about 23.25:23:3.75.

The amount of melanocortin receptor-specific peptide, on a weight-to-weight basis of the pH-sensitive delayed release polymers, may constitute from about 0.1% to about 30%. Preferable the melanocortin receptor-specific peptide constitutes about 1% to about 10%, or about 2% to about 5%, on a weight-to-weight basis, of the pH-sensitive delayed release polymers.

In general, solid forms of melanocortin receptor-specific peptide disposed within a pH-dependent release polymer matrix may be prepared by the methods described herein, or by techniques including, but not limited to, heating, cooling, freeze drying, spray drying, lyophilization, rapid solvent evaporation, solvent recrystallization, microwave-induced precipitation, sonication-induced precipitation, and the like. The particle size of the resulting solid forms, which can vary, for example from about 25 μm or more minimum dimensions to about 1000 μm diameter or lower maximum dimensions, can be controlled, such as by particle-size reduction techniques, including grinding, milling, micronizing or sonication, with or without sieving through suitable screens, or other methods known in the art to select desired ranges of particle size from a set minimum to a set maximum. In one aspect, the particle size is less than about 1000 μm in diameter, or less than about 600 μm in diameter, and more than about 25 μm in damage, or more than about 250 μm in diameter.

### 5.0 Enteric Coatings

In one aspect, the melanocortin receptor-specific peptide, MC1r-specific cyclic peptide or a pharmaceutically acceptable salt thereof is formulated for oral delivery, such as in capsule or tablet form. The peptide may be formulated such that the peptide is in capsule or tablet form encased in an enteric protectant, preferably such that the peptide is not released until the tablet or capsule has transited the stomach, and optionally has further transited all or a portion of the small intestine. In the context of this application it will be understood that the term enteric coating or material refers to a coating or material that will pass through the stomach essentially intact but will rapidly disintegrate in the intestine, preferably but not limited to the large intestine, to release the active peptide drug substance. One enteric coating solution that may be used includes cellulose acetate phthalate, and optionally other ingredients such as ammonium hydroxide, triacetin, ethyl alcohol, methylene blue, and purified water. Cellulose acetate phthalate is a polymer that may be used for enterically coating individual dosage forms such as tablets and capsules and is not soluble in water at a pH of less than about 5.5 to about 6.0. Enteric coatings including cellulose acetate phthalate provide protection against the acidic environment of the stomach, but begin to dissolve in environment of the duodenum (pH of about 6-6.5), and are completely dissolved by the time the dosage form reaches the ileum (pH of about 7-8). In addition to cellulose acetate phthalate, other enteric coating materials are known and may be used with the present invention, including without limitation hydroxypropylmethylethylcellulose succinate, hydroxypropylmethylcellulose phthalate, polyvinyl acetate phthalate, and methacrylic acid-methyl methacrylate copolymer. The enteric coating employed promotes dissolution of the dosage form primarily at a site outside the stomach, and may be selected such that the enteric coating dissolves at a pH of approximately at least 6.0, more preferable at a pH of from about 6.0 to about 8.0. In one preferred aspect, the enteric coating dissolves and breaks down in the proximity of the ileum.

In some embodiments, the melanocortin receptor-specific peptide, MC1r-specific cyclic peptide or a pharmaceutically acceptable salt thereof is formulated in particulate-filled capsule or tablet form with an outer coating, such coating optionally comprising or consisting of a polymer that is stable at low pH, such as pH≤6.0, but which dissolves at a pH greater than about 6.0. The outer coating can further comprise or consist of a polymer that is stable in acid conditions, including in the stomach, but that can dissolve at a higher pH, such as the pH of the lumen of the colon. It is also advantageous and contemplated that the rate of dissolution of the coating can vary depending on the desired release parameters.

The outer coating can, by way of example and not limitation, consist of or include a polymer responsive to and soluble in specified pH ranges, including polymers such as a poly(meth)acrylate. In one aspect, the outer coating consists of or includes one or more polymers or copolymers bearing an anionic group or group that can be converted to an anionic group. In another aspect, the outer coating consists of or includes one more (meth)acrylate copolymers bearing a cationic group or a group that can be converted to a cationic group together with one or more polymers or copolymers bearing an anionic group or group that can be converted to an anionic group. Certain such polymers, copolymers and (meth)acrylate copolymers are taught in U.S. Pat. No. 9,237,760, incorporated herein by reference as if set forth in full. Thus, the enteric coating may be an acrylate polymer such as Eudragit® S100 or Eudragit® L100. Eudragit® S100 dissolves at about pH 7.0 while Eudragit® L100 dissolves at about pH 6.0. Any of the foregoing enteric coatings can be employed with the foregoing formulations, including, without limitations, formulations which comprise a Eudragit® multi-particulate formulation.

In some embodiments, a pharmaceutical composition including a capsule or a tablet may further comprise a sealing or seal coating. This coating may prevent moisture penetration into the tablet. Thus, a seal coating can include a polymer or other material that provides a pharmaceutically acceptable barrier to moisture. Such seal coatings may include polyvinyl alcohol and various combinations of polymers and plasticizers, optionally with a desired pigment.

Other pH-dependent polymers that may be used as enteric coatings include, but are not limited to, enteric cellulose derivatives such as hydroxypropyl methylcellulose phthalate, hydroxypropyl methylcellulose acetate succinate, cellulose acetate phthalate; natural resins such as shellac and zein; enteric acetate derivates such as polyvinylacetate phthalate, cellulose acetate phthalate, acetaldehyde dimethylcellulose acetate; and various polymethacrylate-based polymers in addition to those disclosed above. The pH-dependent enteric coating may also comprise combinations of two or more pH-dependent polymers, including any of the foregoing.

### 6.0 Peptides Utilized in the Present Invention

In one aspect, the invention utilizes a cyclic peptide which contains a core sequence derived from His-Phe-Arg within the cyclic portion, but not including Trp within the core portion, and where Trp, or a derivative or mimetic thereof (defined as an amino acid residue with a side chain including at least one aryl or heteroaryl, including but not limited to Nal 1 or Nal 2), is the amino acid residue immediately outside the cyclic portion on the C-terminus side. In one aspect, the sequence His-Phe-Arg-Xaa⁶-Trp (SEQ ID NO:1) is employed, where Xaa⁶ is an amino acid wherein the side chain thereof forms a cyclic bridge with either the side chain of another amino acid of the peptide.

The core sequence derived from His-Phe-Arg-Xaa⁶-Trp (SEQ ID NO:1) may include a number of substitutions. The His position may be His, or may be a substituted or unsubstituted Pro or an amino acid with a side chain including at least one primary amine, secondary amine, alkyl, cycloalkyl, cycloheteroalkyl, aryl, heteroaryl, alcohol, ether, sulfide, sulfone, sufoxide, carbomyl or carboxyl. Substituted Pro includes, but is not limited to, amino acids such as Hyp, Hyp(Bzl), Pro(4R-Bzl) or Pro(4R—NH₂). The Phe position may be Phe, but is most typically substituted or unsubstituted D-Phe, D-Nal 1, D-Nal 2 or an amino acid with a side chain including pyridyl. The Arg position may be Arg, Lys, Orn, Dab or Dap, or a substituted or unsubstituted Pro, or Cit, or may be an amino acid with a side chain including at least one primary amine, secondary amine, guanidine, urea, alkyl, cycloalkyl, cycloheteroalkyl, aryl, heteroaryl, or ether. Xaa⁶ may be an amino acid with a side chain include a primary amine, such as Lys, Orn, Dab, Dap, an amino acid with a carboxyl group, such as Asp, Glu or hGlu, or an amino acid with a disulfide group, such as Cys or Pen, all depending on the nature of the cyclic bridge. The Trp position may be an amino acid with a side chain including at least one substituted or unsubstituted aryl or heteroaryl, such as Trp, Nal 1 or Nal 2.

In one aspect, the invention utilizes a formulation comprising a cyclic peptide of formula (I):

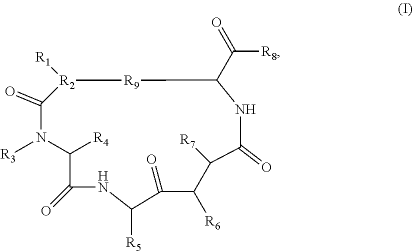

including all enantiomers, stereoisomers or diastereoisomers thereof, or a pharmaceutically acceptable salt of any of the foregoing,

wherein:

R₁ is —H, —NH—R₁₀, —NH—R₁₀-R₁₁ or —NH—R₁₁;

R₂ is —CH— or —N—;

R₃ is —H, —CH₃ or —CH₂—, and if it is —CH₂— forms with R₄ a ring of the general structure

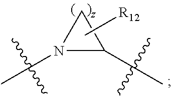

R₄ is —H, —(CH₂)_{z}— if R₃ is —CH₂—, and if it is —(CH₂)_{z}— forms the ring with R₃, wherein any H in —(CH₂)_{z}— is optionally substituted with R₁₂, or R₄ is —(CH₂)_{w}—R₁₃—(CH₂)_{w}—R₁₄, wherein any H in either (CH₂)_{w} is optionally substituted with —(CH₂)_{w}—CH₃;

R₅ is —(CH₂)_{w}—R₁₅;

R₆ is —H, —CH₃ or —CH₂—, and if it is —CH₂— forms with R₇ a ring of the general structure

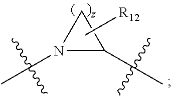

R₇ is —(CH₂)_{z}— if R₆ is —CH₂—, and if it is —(CH₂)_{z}— forms the ring with R₆, or R₇ is —(CH₂)_{w}—R₁₆;

R₈ is —R₁₇-R₁₈ or —R₁₈;

R₉ is
—(CH₂)ₓ—C(═O)—NH—(CH₂)_{y}—,
—(CH₂)ₓ—NH—C(═O)—(CH₂)_{y}—,
—(CH₂)ₓ—C(═O)—(CH₂)_{z}—C(═O)—(CH₂)_{y}—,
—(CH₂)ₓ—C(═O)—NH—C(═O)—(CH₂)_{y}—,
   —(CH₂)ₓ—NH—C(═O)—NH—(CH₂)_{y}—,
   —(CH₂)ₓ—NH—C(═O)—(CH₂)_{z}—C(═O)—NH—(CH₂)_{y}—, or
   —(CH₂)ₓ—S—S—(CH₂)_{y}—;

R₁₀ is from one to three amino acid residues;

R₁₁ is H or a C₁ to C₁₇ acyl group, wherein the C₁ to C₁₇ comprises a linear or branched alkyl, cycloalkyl, alkylcycloalkyl, aryl or alkylaryl;

R₁₂ is optionally present, and if present is independently in each instance —R₁₃—(CH₂)_{w}—R₁₄;

R₁₃ is optionally present, and if present is independently in each instance
—O—,
—S—,
—NH—,
—S(═O)₂—,
—S(═O)—,
—S(═O)₂—NH—,
—NH—S(═O)₂—,
—C(═O)—,
—C(═O)—O—,
—O—C(═O)—,
—NH—C(═O)—O—,
—O—C(═O)—NH—,
—NH—C(═O)—, or
—C(═O)—NH—;

R₁₄ is independently in each instance —H, —CH₃, —N(R₁₉ₐ)(R_{19b}), —NH—(CH₂)_{z}—N(R₁₉ₐ)(R_{19b}), —NH—CH(═NH)—N(R₁₉ₐ)(R_{19b}), —NH—CH(═O)—N(R₁₉ₐ)(R_{19b}), —O(R₁₉ₐ), —(R₁₉ₐ)(R_{19b}), —S(═O)₂(R₁₉ₐ), —C(═O)—O(R₁₉ₐ),

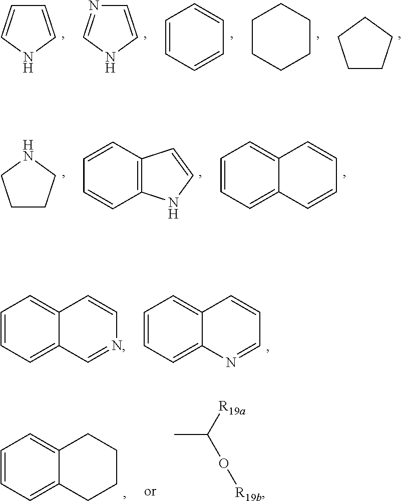

wherein any ring in R₁₄ is optionally substituted with one or more ring substituents, and when one or more substituents are present, are the same or different and independently hydroxyl, halogen, sulfonamide, alkyl, —O-alkyl, aryl, —O-aryl, C(═O)—OH, or C(═O)—N(R₁₉ₐ)(R_{19b});

R₁₅ is phenyl, naphthyl or pyridyl, optionally substituted with one or more substituents independently selected from halo, (C₁-C₁₀)alkyl-halo, (C₁-C₁₀)alkoxy, (C₁-C₁₀)alkylthio, aryl, aryloxy, nitro, nitrile, sulfonamide, amino, monosubstituted amino, disubstituted amino, hydroxy, carboxy, and alkoxy-carbonyl;

R₁₆ is —H, —N(R₁₉ₐ)(R_{19b}), —NH—(CH₂)_{z}—N(R₁₉ₐ)(R_{19b}), —NH—CH(═NH)—N(R₁₉ₐ)(R_{19b}), —NH—CH(═O)—N(R₁₉ₐ)(R_{19b}), —O(R₁₉ₐ), a linear or branched C₁ to C₁₇ alkyl chain, —C(═O)—N(R₁₉ₐ)(R_{19b}), —S(═O)₂(R₁₉ₐ),

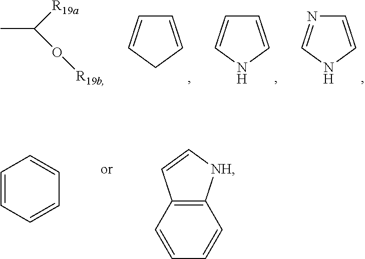

wherein any ring is optionally substituted with one or more optional ring substituents, and when one or more substituents are present, are the same or different and independently hydroxyl, halogen, sulfonamide, alkyl, —O-alkyl, aryl, aralkyl, O-aralkyl, or —O-aryl;

R₁₇ is from one to three amino acid residues;

R₁₈ is —OH, —N(R₁₉ₐ)(R_{19b}), —N(R₁₉ₐ)(CH₂)_{w}—(C₁-C₇)cycloalkyl, or —O—(CH₂)_{w}—(C₁-C₇)cycloalkyl;

R₁₉ₐ and R_{19b} are each independently H or a C₁ to C₄ linear or branched alkyl chain;

w is in each instance independent 0 to 5;

x is 1 to 5;

y is 1 to 5; and

z is in each instance independently 1 to 5.

In the cyclic peptide of formula (I) R₁₇ may be a single amino acid residue of the formula

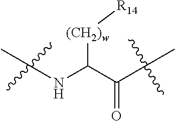

wherein R₂₀ is

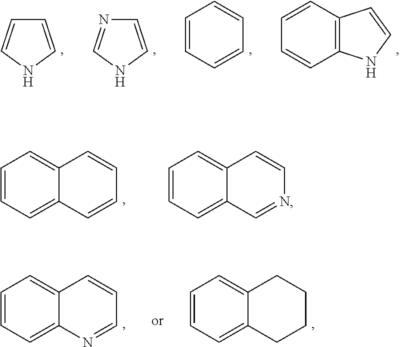

optionally substituted with one or more ring substituents, and when one or more are present, are the same or different and independently hydroxyl, halogen, sulfonamide, alkyl, —O-alkyl, aryl, or —O-aryl.

In another aspect, the invention utilizes a cyclic peptide of formula (II):

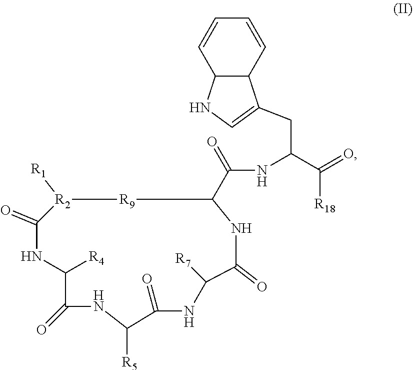

wherein variables are as assigned for formula (I).

In another aspect, the invention utilizes a cyclic peptide of formula (III):

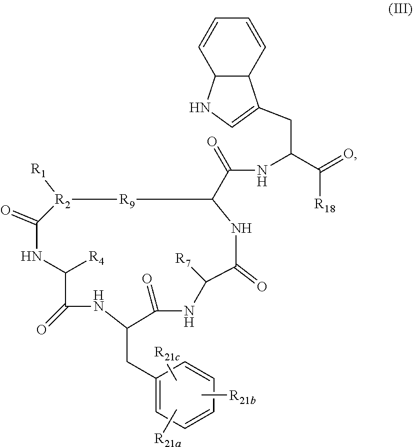

wherein R₂₁ₐ, R_{21b} and R_{21c} are independently in each instance hydrogen, halo, (C₁-C₁₀)alkyl-halo, (C₁-C₁₀)alkyl, (C₁-C₁₀)alkoxy, (C₁-C₁₀)alkylthio, aryl, aryloxy, nitro, nitrile, sulfonamide, amino, monosubstituted amino, disubstituted amino, hydroxy, carboxy, or alkoxy-carbonyl, and all other variables are as assigned for formula (I).

In another aspect, the invention utilizes a cyclic peptide of formula (IV):

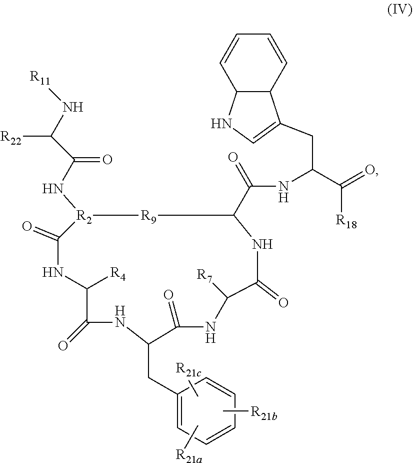

wherein R₂₂ is H or a C₁ to C₉ linear or branched alkyl, cycloalkyl, alkylcycloalkyl, aryl or alkylaryl;

R₂₁ₐ, R_{21b} and R_{21c} are as defined for formula (III); and

all other variables are as assigned for formula (I).

In another aspect, the invention utilizes a cyclic peptide of formula (V):

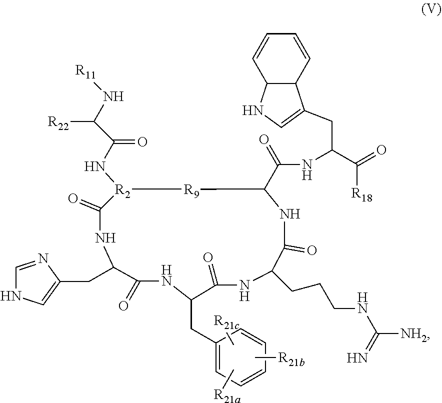

wherein variables are as assigned for the cyclic peptide of formula (IV).

In another aspect, the invention utilizes a cyclic peptide of formula (VI):

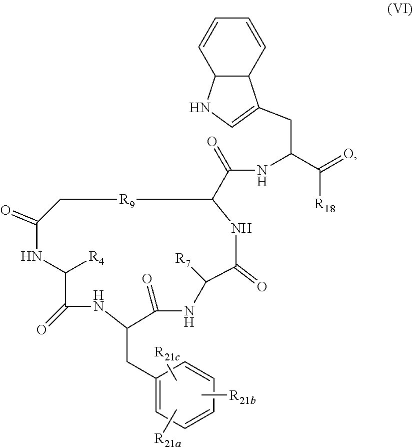

wherein variables are as assigned for the cyclic peptide of formula (III).

In the cyclic peptide of formula (I), R₉ may be —(CH₂)ₓ—C(═O)—NH—(CH₂)_{y}— where x is 4 and y is 3, where x is 3 and y is 2, or where x is 2 and y is 1. Alternatively, R₉ may be —(CH₂)ₓ—NH—C(═O)—(CH₂)_{y}— where x is 1 and y is 2, where x is 2 and y is 3, or where x is 3 and y is 4.

In the cyclic peptide of formula (I), R₃ may form with R₄ a ring of the general structure

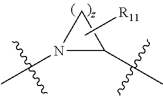

where z is 3.

In the cyclic peptide of formula (I), R₁₇ may be a single amino acid residue of the formula

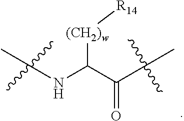

The invention thus in one aspect may utilize a cyclic peptide of formula (VII):

Z-Xaa¹-Xaa²-Xaa³-Xaa⁴-Xaa⁶-Xaa⁶-Xaa⁷-Y (VII)

or a pharmaceutically acceptable salt thereof, wherein:

Z is H or an N-terminal group;

Xaa¹ is optionally present, and if present is from one to three L- or D-isomer amino acid residues;

Xaa² and Xaa⁶ are L- or D-isomer amino acids wherein the side chains thereof comprise a cyclic bridge;

Xaa³ is L- or D-Pro, optionally substituted with hydroxyl, halogen, sulfonamide, alkyl, —O-alkyl, aryl, alkyl-aryl, alkyl-O-aryl, alkyl-O-alkyl-aryl, or —O-aryl, or Xaa³ is an L- or D-isomer of an amino acid with a side chain including at least one primary amine, secondary amine, alkyl, cycloalkyl, cycloheteroalkyl, aryl, heteroaryl, ether, sulfide, or carboxyl;

Xaa⁴ is an L- or D-isomer amino acid with a side chain including phenyl, naphthyl or pyridyl, optionally wherein the ring is substituted with one or more substituents independently selected from halo, (C₁-C₁₀)alkyl-halo, (C₁-C₁₀)alkyl, (C₁-C₁₀)alkoxy, (C₁-C₁₀)alkylthio, aryl, aryloxy, nitro, nitrile, sulfonamide, amino, monosubstituted amino, disubstituted amino, hydroxy, carboxy, and alkoxy-carbonyl;

Xaa⁵ is L- or D-Pro or Xaa⁵ is an L- or D-isomer amino acid with a side chain including at least one primary amine, secondary amine, guanidine, urea, alkyl, cycloalkyl, cycloheteroalkyl, aryl, heteroaryl, or ether;

Xaa⁷ is optionally present, and if present is from one to three L- or D-isomer amino acid residues; and

Y is a C-terminal group.

In one aspect, Xaa⁴ may be D-Phe, optionally substituted with one or more substituents independently selected from halo, (C₁-C₁₀)alkyl-halo, (C₁-C₁₀)alkyl, (C₁-C₁₀)alkoxy, (C₁-C₁₀)alkylthio, aryl, aryloxy, nitro, nitrile, sulfonamide, amino, monosubstituted amino, disubstituted amino, hydroxy, carboxy, and alkoxy-carbonyl.

In another aspect, one of Xaa² and Xaa⁶ may be an L- or D-isomer of Asp, hGlu or Glu and the other of Xaa² and Xaa⁶ is an L- or D-isomer of Lys, Orn, Dab or Dap. In an alternative aspect, each of Xaa² and Xaa⁶ may be Cys, D-Cys, Pen or D-Pen.

In another aspect, Xaa¹ may be an amino acid with a side chain including a linear or branched alkyl, cycloalkyl, cycloheteroalkyl, aryl or heteroaryl.

In another aspect, Xaa⁷ may be an amino acid with a side chain including at least one aryl or heteroaryl, optionally substituted with one or more ring substituents, and when one or more substituents are present, are the same or different and independently hydroxyl, halogen, sulfonamide, alkyl, —O-alkyl, aryl, or —O-aryl.

In another aspect, the N-terminal group may be a C₁ to C₁₇ acyl group, wherein the C₁ to C₁₇ comprises a linear or branched alkyl, cycloalkyl, alkylcycloalkyl, aryl or alkylaryl, a linear or branched C₁ to C₁₇ alkyl, aryl, heteroaryl, alkene, alkenyl, or aralkyl chain or an N-acylated linear or branched C₁ to C₁₇ alkyl, aryl, heteroaryl, alkene, alkenyl, or aralkyl chain.

In another aspect, Y may be a hydroxyl, an amide, or an amide substituted with one or two linear or branched C₁ to C₁₇ alkyl, cycloalkyl, aryl, alkyl cycloalkyl, aralkyl, heteroaryl, alkene, alkenyl, or aralkyl chains.

The invention thus provides in another aspect a cyclic peptide of formula (VII) defined as above, but wherein

Xaa⁴ is D-Phe, optionally substituted with one or more substituents independently selected from halo, (C₁-C₁₀)alkyl-halo, (C₁-C₁₀)alkyl, (C₁-C₁₀)alkoxy, (C₁-C₁₀)alkylthio, aryl, aryloxy, nitro, nitrile, sulfonamide, amino, monosubstituted amino, disubstituted amino, hydroxy, carboxy, and alkoxy-carbonyl;

Xaa⁵ is an L- or D-isomer of Arg, Lys, Orn, Dab or Dap; and

Xaa⁷ is an L- or D-isomer of Trp, Nal 1 or Nal 2.

In the foregoing, in one aspect Xaa³ may be an L- or D-isomer of His, and in another aspect Z may be a C₁ to C₁₇ acyl group and Xaa¹ may be an L- or D-isomer of Nle.

In the foregoing, and in formula (I), substituted Pro may be, for example, Hyp, Hyp(Bzl), Pro(4-Bzl), and Pro(4-NH₂).

The peptides encompassed within formulas (I) through (VII) contain one or more asymmetric elements such as stereogenic centers, stereogenic axes and the like, so that the peptides encompassed within formula (I) can exist in different stereoisomeric forms. For both specific and generically described peptides, including the peptides encompassed within formulas (I) through (VII), all forms of isomers at all chiral or other isomeric centers, including enantiomers and diastereomers, are intended to be covered herein. The peptides of the invention each include multiple chiral centers, and may be used as a racemic mixture or an enantiomerically enriched mixture, in addition to use of the peptides of the invention in enantiopure preparations. Typically, the peptides of the invention will be synthesized with the use of chirally pure reagents, such as specified L- or D-amino acids, using reagents, conditions and methods such that enantiomeric purity is maintained, but it is possible and contemplated that racemic mixtures may be made. Such racemic mixtures may optionally be separated using well-known techniques and an individual enantiomer may be used alone. In cases and under specific conditions of temperature, solvents and pH wherein peptides may exist in tautomeric forms, each tautomeric form is contemplated as being included within this invention whether existing in equilibrium or predominantly in one form. Thus a single enantiomer of a peptide of formula (I), which is an optically active form, can be obtained by asymmetric synthesis, synthesis from optically pure precursors, or by resolution of the racemates.

The invention is further intended to include prodrugs of the present peptides, which on administration undergo chemical conversion by metabolic processes before becoming active pharmacological peptides. In general, such prodrugs will be functional derivatives of the present peptides, which are readily convertible in vivo into a peptide of formula (I) through (VII). Prodrugs are any covalently bonded compounds, which release the active parent peptide drug of formula (I) through (VII) in vivo. Conventional procedures for the selection and preparation of suitable prodrug derivatives are described, for example, in “Design of Prodrugs”, ed. H. Bundgaard, Elsevier, 1985. Typical examples of prodrugs have biologically labile protecting groups on a functional moiety, such as for example by esterification of hydroxyl, carboxyl or amino functions. Thus by way of example and not limitation, a prodrug includes peptides of formula (I) wherein an ester prodrug form is employed, such as, for example, lower alkyl esters of an R group of formula (I), such as where R is —OH, which lower alkyl esters may include from 1-8 carbons in an alkyl radical or aralkyl esters which have 6-12 carbons in an aralkyl radical. Broadly speaking, prodrugs include compounds that can be oxidized, reduced, aminated, deaminated, hydroxylated, dehydroxylated, hydrolyzed, dehydrolyzed, alkylated, dealkylated, acylated, deacylated, phosphorylated or dephosphorylated to produce an active parent peptide drug of formula (I) in vivo.

The subject invention also includes peptides which are identical to those recited in formula (I) through (VI), but for the fact that one or more atoms depicted in formula (I) through (VI) are replaced by an atom having an atomic mass or mass number different from the atomic mass or mass number usually found in nature. Examples of isotopes that can be incorporated into compounds of the invention include isotopes of hydrogen, carbon, nitrogen and oxygen, such as ²H, ³H, ¹³C, ¹⁴C, ¹⁵N, ¹⁸O and ¹⁷O, respectively. Peptides of the present invention and pharmaceutically acceptable salts or solvates of said compounds which contain the aforementioned isotopes and/or other isotopes of other atoms are within the scope of this invention. Certain isotopically-labeled compounds of the present invention, for example those into which radioactive isotopes such as ³H and ¹⁴C are incorporated, may have use in a variety of assays, such as in drug and/or substrate tissue distribution assays. Substitution with heavier isotopes, such as substitution of one or more hydrogen atoms with deuterium (²H), can provide pharmacological advantages in some instances, including increased metabolic stability. Isotopically labeled peptides of formula (I) through (VI) can generally be prepared by substituting an isotopically labeled reagent for a non-isotopically labeled reagent.

### 7.0 Methods of Making Peptides Utilized in the Invention

In general, the peptides of the present invention may be synthesized by solid-phase synthesis and purified according to methods known in the art. Any of a number of well-known procedures utilizing a variety of resins and reagents may be used to prepare the peptides of the present invention.

The cyclic peptides of the present invention may be readily synthesized by known conventional procedures for the formation of a peptide linkage between amino acids. Such conventional procedures include, for example, any solution phase procedure permitting a condensation between the free alpha amino group of an amino acid or residue thereof having its carboxyl group and other reactive groups protected and the free primary carboxyl group of another amino acid or residue thereof having its amino group or other reactive groups protected. In a preferred conventional procedure, the cyclic peptides of the present invention may be synthesized by solid-phase synthesis and purified according to methods known in the art. Any of a number of well-known procedures utilizing a variety of resins and reagents may be used to prepare the peptides of the present invention.

The process for synthesizing the cyclic peptides may be carried out by a procedure whereby each amino acid in the desired sequence is added one at a time in succession to another amino acid or residue thereof or by a procedure whereby peptide fragments with the desired amino acid sequence are first synthesized conventionally and then condensed to provide the desired peptide. The resulting peptide is then cyclized to yield a cyclic peptide of the invention.

Solid phase peptide synthesis methods are well known and practiced in the art. In such methods the synthesis of peptides of the invention can be carried out by sequentially incorporating the desired amino acid residues one at a time into the growing peptide chain according to the general principles of solid phase methods. These methods are disclosed in numerous references, including Merrifield, R. B., “Solid phase synthesis (Nobel lecture),” Angew Chem 24:799-810 (1985) and Barany et al., The Peptides, Analysis, Synthesis and Biology, Vol. 2, Gross, E. and Meienhofer, J., Eds. Academic Press 1-284 (1980).

In chemical syntheses of peptides, reactive side chain groups of the various amino acid residues are protected with suitable protecting groups, which prevent a chemical reaction from occurring at that site until the protecting group is removed. Also common is the protection of the alpha amino group of an amino acid residue or fragment while that entity reacts at the carboxyl group, followed by the selective removal of the alpha amino protecting group to allow a subsequent reaction to take place at that site. Specific protecting groups have been disclosed and are known in solid phase synthesis methods and solution phase synthesis methods.

Alpha amino groups may be protected by a suitable protecting group, including a urethane-type protecting group, such as benzyloxycarbonyl (Z) and substituted benzyloxycarbonyl, such as p-chlorobenzyloxycarbonyl, p-nitrobenzyloxycarbonyl, p-bromobenzyloxycarbonyl, p-biphenyl-isopropoxycarbonyl, 9-fluorenylmethoxycarbonyl (Fmoc) and p-methoxybenzyloxycarbonyl (Moz) and aliphatic urethane-type protecting groups, such as t-butyloxycarbonyl (Boc), diisopropylmethoxycarbonyl, isopropoxycarbonyl, and allyloxycarbonyl (Alloc). Fmoc is preferred for alpha amino protection.

Guanidino groups may be protected by a suitable protecting group, such as nitro, p-toluenesulfonyl (Tos), Z, pentamethylchromanesulfonyl (Pmc), adamantyloxycarbonyl, pentamethyldihydrobenzofuran-5-sulfonyl (Pbf) and Boc. Pbf and Pmc are preferred protecting groups for Arg.

The peptides of the invention described herein were prepared using solid phase synthesis, such as by means of a Symphony Multiplex Peptide Synthesizer (Rainin Instrument Company) automated peptide synthesizer, using programming modules as provided by the manufacturer and following the protocols set forth in the manufacturer's manual.

Solid phase synthesis is commenced from the C-terminal end of the peptide by coupling a protected alpha amino acid to a suitable resin. Such starting material is prepared by attaching an alpha amino-protected amino acid by an amide linkage to 9-Fmoc-aminoxanthen-3-yloxy-Merrifield resin (Sieber Amide resin) or to 4-(2′,4′-Dimethoxyphenyl-Fmoc-aminomethyl)phenoxy resin (Rink Amide resin), by an ester linkage to a p-benzyloxybenzyl alcohol (Wang) resin, a 2-chlorotrityl chloride resin or an oxime resin, or by other means well known in the art. The resins are carried through repetitive cycles as necessary to add amino acids sequentially. The alpha amino Fmoc protecting groups are removed under basic conditions. Piperidine, piperazine, diethylamine, or morpholine (20-40% v/v) in N,N-dimethylformamide (DMF) may be used for this purpose.

Following removal of the alpha amino protecting group, the subsequent protected amino acids are coupled stepwise in the desired order to obtain an intermediate, protected peptide-resin. The activating reagents used for coupling of the amino acids in the solid phase synthesis of the peptides are well known in the art. After the peptide is synthesized, if desired, the orthogonally protected side chain protecting groups may be removed using methods well known in the art for further derivatization of the peptide.

Typically, orthogonal protecting groups are used as appropriate. For example, the peptides of the invention contain multiple amino acids with an amino group-containing side chain. In one aspect, an Allyl-Alloc protection scheme is employed with the amino acids forming a lactam bridge through their side chains, and orthogonal protecting groups, cleavable under different reactive conditions, use for other amino acids with amino group-containing side chains. Thus, for example, Fmoc-Lys(Alloc)-OH, Fmoc-Orn(Alloc)-OH, Fmoc-Dap(Alloc)-OH, Fmoc-Dab(Alloc)-OH, Fmoc-Asp(OAII)-OH or Fmoc-Glu(OAII)-OH amino acids can be employed for the positions forming a lactam bridge upon cyclization, while other amino acids with amino group-containing side chains have a different and orthogonal protecting group, such as with Fmoc-Arg(Pbf)-OH, Fmoc-Lys(Boc)-OH, Fmoc-Dab(Boc)-OH or the like. Other protecting groups may be similarly employed; by way of example and not limitation, Mtt/OPp (4-methyltrityl/2-phenylisopropyl) can be employed with the side chains forming a lactam bridge upon cyclization, with orthogonal protecting groups being utilized for other positions that are not cleavable using conditions suitable for cleavage of Mtt/OPp.

Reactive groups in a peptide can be selectively modified, either during solid phase synthesis or after removal from the resin. For example, peptides can be modified to obtain N-terminus modifications, such as acetylation, while on resin, or may be removed from the resin by use of a cleaving reagent and then modified. Similarly, methods for modifying side chains of amino acids are well known to those skilled in the art of peptide synthesis. The choice of modifications made to reactive groups present on the peptide will be determined, in part, by the characteristics that are desired in the peptide.

In the peptides of the present invention, in one embodiment the N-terminus group is modified by introduction of an N-acetyl group. In one aspect, a method is employed wherein after removal of the protecting group at the N-terminal, the resin-bound peptide is reacted with acetic anhydride in N,N-dimethylformamide (DMF) in the presence of an organic base, such as pyridine. Other methods of N-terminus acetylation are known in the art, including solution phase acetylation, and may be employed.

The peptide can, in one embodiment, be cyclized prior to cleavage from the peptide resin. For cyclization through reactive side chain moieties, the desired side chains are deprotected, and the peptide suspended in a suitable solvent and a cyclic coupling agent added. Suitable solvents include, for example DMF, dichloromethane (DCM) or 1-methyl-2-pyrrolidone (NMP). Suitable cyclic coupling reagents include, for example, 2-(1H-benzotriazol-1-yl)-1,1,3,3-tetramethyluronium tetrafluoroborate (TBTU), 2-(1H-benzotriazol-1-yl)-1,1,3,3-tetramethyluronium hexafluorophosphate (HBTU), benzotriazole-1-yl-oxy-tris(dimethylamino)phosphoniumhexafluorophosphate (BOP), benzotriazole-1-yl-oxy-tris(pyrrolidino)phosphoniumhexafluorophosphate (PyBOP), 2-(7-aza-1H-benzotriazol-1-yl)-1,1,3,3-tetramethyluronium tetrafluoroborate (TATU), 2-(2-oxo-1(2H)-pyridyl)-1,1,3,3-tetramethyluronium tetrafluoroborate (TPTU) or N,N′-dicyclohexylcarbodiimide/1-hydroxybenzotriazole (DCCI/HOBt). Coupling is conventionally initiated by use of a suitable base, such as N,N-diisopropylethylamine (DIPEA), sym-collidine or N-methylmorpholine (NMM).

For peptides with a non-lactam cyclic bridge, such as peptides containing the bridge:

—(CH₂)ₓ—NH—C(═O)—(CH₂)_{z}—C(═O)—NH—(CH₂)_{y}—,

where x, y and z are each independently 1 to 5, the peptides may be made using solid phase synthesis employing a side-chain protected diamine amino acid for the positions to be cyclized. Particularly preferred in such positions are Dap, Dab or Lys, preferably with an amine protecting group such as Alloc, Mtt, Mmt (methoxytrityl), Dde (1-(4,4-dimethyl-2,6-dioxocyclohex-1-ylidene))ethyl), ivDde (1-(4,4-dimethyl-2,6-dioxocyclohex-1-ylidene)-3-methylbutyl) or any other orthogonally cleavable protecting group. Typically, one side chain protecting group is removed first, such as removal of Mtt using 2% TFA in dichloromethane. Following washing of the resin, the resulting resin-bound unprotected amine is acylated, such as with a 0.5 M solution of a cyclic anhydride such as succinic anhydride or glutaric anhydride in dichloromethane/pyridine 1:1. Following additional wash steps, the orthogonally cleavable protecting group of the second diamino amino acid is cleaved, such as removal of Alloc using tetrakis(triphenylphosphine)palladium(0) and phenyl silane in dichloromethane. After washing with dichloromethane and DMF the resin-bound peptide is cyclized using standard coupling reagents such as TBTU and a base. Alternatively, an ivDde protected resin-bound diamino amino acid can be deprotected using a solution of 5% of hydrazine in DMF, and after washing with DMF the resulting resin bound amine can either be acylated with a cyclic anhydride or can be cyclized with a resin bound carboxylic acid.

The cyclized peptides can then be cleaved from solid phase, using any suitable reagent, such as ethylamine in DCM or various combinations of agents, such as trifluoroacetic acid (TFA), tri-isopropylsilane (TIS), dimethoxybenezene (DMB), water and the like. The resulting crude peptide is dried and remaining amino acid side chain protecting groups, if any, are cleaved using any suitable reagent, such as (TFA) in the presence of water, TIS, 2-mercaptopethane (ME), and/or 1,2-ethanedithiol (EDT). The final product is precipitated by adding cold ether and collected by filtration. Final purification is by reverse phase high performance liquid chromatography (RP-HPLC), using a suitable column, such as a C₁₈ column, or other methods of separation or purification, such as methods based on the size or charge of the peptide, can also be employed. Once purified, the peptide can be characterized by any number of methods, such as high performance liquid chromatograph (HPLC), amino acid analysis, mass spectrometry, and the like.

For peptides of the present invention which have a C-terminus substituted amide derivative or N-alkyl group, synthesis may proceed by solid phase synthesis commenced from the C-terminal end of the peptide by coupling a protected alpha amino acid to a suitable resin. Such methods for preparing substituted amide derivatives on solid-phase have been described in the art. See, for example, Barn, D. R., et al., “Synthesis of an array of amides by aluminum chloride assisted cleavage on resin bound esters,” Tetrahedron Letters, 37:3213-3216 (1996); DeGrado, W. F. and Kaiser E. T., “Solid-phase synthesis of protected peptides on a polymer bound oxime: Preparation of segments comprising the sequences of a cytotoxic 26-peptide analogue,” J. Org. Chem., 47:3258-3261 (1982). Such a starting material can be prepared by attaching an alpha amino-protected amino acid by an ester linkage to a p-benzyloxybenzyl alcohol (Wang) resin or an oxime resin by well known means. The peptide chain is grown with the desired sequence of amino acids, the peptide cyclized and the peptide-resin treated with a solution of appropriate amine (such as methyl amine, dimethyl amine, ethylamine, and so on). Peptides employing a p-benzyloxybenzyl alcohol (Wang) resin may be cleaved from resin by aluminum chloride in DCM, and peptides employing an oxime resin may be cleaved by DCM. Another method to prepare a peptide with a C-terminus substituted amide is to attach an alkyl amine by reductive amination to a formyl resin, such as 4-(4-Formyl-3-methoxyphenoxy)butyryl-AM resin (FMPB AM resin), and then sequentially incorporate desired amino acid residues utilizing general principles of solid phase synthesis.

While synthesis has been described primarily with reference to solid phase Fmoc chemistry, it is to be understood that other chemistries and synthetic methods may be employed to make the cyclic peptides of the invention, such as by way of example and not limitation, methods employing Boc chemistry, solution chemistry, and other chemistries and synthetic methods.

### 8.0 Tests and Assays Employed in Evaluation of Peptides Utilized in the Present Invention

The melanocortin receptor-specific peptides utilized in the present invention may be tested by a variety of assay systems and animal models to determine binding, functional status and efficacy.

8.1 Competitive Inhibition Assay using [I¹²⁵]-NDP-α-MSH.

A competitive inhibition binding assay was performed using membrane homogenates prepared from HEK-293 cells that express recombinant hMC1r or hMC4r (in each instance where the h prefix refers to human), or alternatively membrane homogenates from B16-F10 mouse melanoma cells containing endogenous murine MC1r. In the examples that follow, all MC1r and MC4r values are for human recombinant receptors, unless otherwise noted. Assays were performed in 96 well polypropylene round-bottom plates (VWR catalog number 12777-030). Membrane homogenates were incubated with 0.1 nM [I¹²⁵]-NDP-α-MSH (Perkin Elmer) and increasing concentrations of test peptides of the present invention in buffer containing 25 mM HEPES buffer (pH 7.5) with 100 mM NaCl, 2 mM CaCl₂, 2 mM MgCl₂, 0.3 mM 1,10-phenanthroline, and 0.2% bovine serum albumin. After incubation for 90 minutes at 37° C., the assay mixture was filtered onto GF/B Unifilter plates (Perkin-Elmer catalog number 6005177) and washed with 3 mL of ice-cold buffer per well. Filters were air dried and 35 μL of scintillation cocktail added to each well. Plates were counted in a Microbeta counter for bound radioactivity. Non-specific binding was measured by inhibition of binding of [I¹²⁵]-NDP-α-MSH in the presence of 1 μM NDP-α-MSH. Maximal specific binding (100%) was defined as the difference in radioactivity (cpm) bound to cell membranes in the absence and presence of 1 μM NDP-α-MSH. Each assay was conducted in duplicate and the actual mean values are described, with results less than 0% reported as 0%. Ki values for peptides of the present invention were determined using Graph-Pad Prism® curve-fitting software.

8.2 Assay for Agonist Activity.

Accumulation of intracellular cAMP was examined as a measure of the ability of the peptides of the present invention to elicit a functional response in a human melanoma cell line, HBL, that express hMC1r (see Kang, L., et al., “A selective small molecule agonist of MC1r inhibits lipopolysaccharide-induced cytokine accumulation and leukocyte infiltration in mice,” J. Leuk. Biol. 80:897-904 (2006)) or HEK-293 cells that express hMC4r. Confluent HBL cells that express hMC1r or HEK-293 cells that express recombinant hMC4r were detached from culture plates by incubation in enzyme-free cell dissociation buffer. Dispersed cells were suspended in Earle's Balanced Salt Solution containing 10 mM HEPES (pH 7.5), 1 mM MgCl₂, 1 mM glutamine, 0.5% albumin and 0.3 mM 3-isobutyl-1-methyl-xanthine (IBMX), a phosphodiesterase inhibitor. The cells were plated in 96-well plates at a density of 0.4×10⁵ cells per well for HBL cells and 0.5×10⁵ cells per well for HEK-293 cells and pre-incubated for 10 minutes. Cells were exposed for 15 minutes at 37° C. to peptides of the present invention dissolved in DMSO (final DMSO concentration of 1%) at a concentration range of 0.05-5000 nM in a total assay volume of 200 μL. NDP-α-MSH was used as the reference agonist. cAMP levels were determined by an HTRF® cAMP cell-based assay system from Cisbio Bioassays utilizing cryptate-labeled anti-cAMP and d2-labeled cAMP, with plates read on a Perkin-Elmer Victor plate reader at 665 and 620 nM. Data analysis was performed by nonlinear regression analysis with Graph-Pad Prism® software. Maximum efficacy (Eₘₐₓ) values were determined for each test peptide of the present invention, compared to that achieved by the reference melanocortin agonist NDP-α-MSH.

### 9.0 Examples of Peptides Utilized in the Invention

Peptides of the following structures were synthesized and averaged MC1r and MC4r Ki values were determined as indicated. Ki values were determined using [I¹²⁵]-NDP-α-MSH. All results are expressed in nM except for Eₘₐₓ values, which are percentage values. Peptides with the captioned primary sequence were synthesized and purified as described in Section 7 above, with the resulting peptide having the structure depicted. After synthesis and purification, peptides were tested as described in Section 8 above, with the results as shown.

| 9.1 | Ac-Nle-cyclo(Glu-His-D-Phe-Arg-Lys)-Trp-NH₂ (SEQ ID NO: 4) | |
|---|---|---|
| | 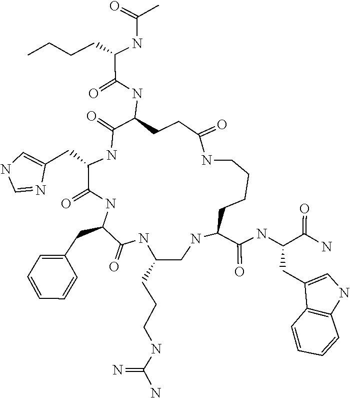 | |
| | Assay | Result |
| | MC-4 Ki (average) | 45 |
| | MC-1 Ki (average) | 0.01 |
| | MC-1 EC₅₀ (average; cAMP HBL) | 0.007 |
| | MC-1 Eₘₐₓ (average; cAMP HBL) | 97% |
| 9.2 | Ac-Nle-cyclo(Glu-His-D-Phe-Arg-Dab)-Trp-NH₂ (SEQ ID NO: 5) | |
| | 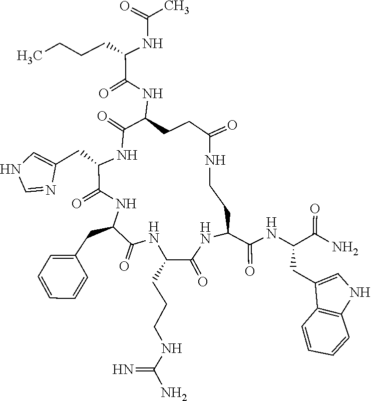 [SMILES: CCCC[C@H](NC(C)=O)C(=O)N[C@H]1CCC(=O)NCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 110 |
| | MC-1 Ki (average) | 0.012 |
| | MC-1 EC₅₀ (average; cAMP HBL) | 0.006 |
| | MC-1 Eₘₐₓ (average; cAMP HBL) | 95% |
| 9.3 | Ac-Nle-cyclo(Glu-His-D-Phe-Arg-Dap)-Trp-NH₂ (SEQ ID NO: 6) | |
| | 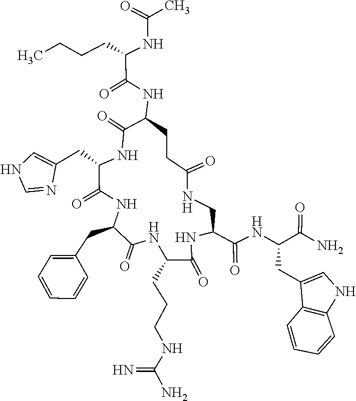 [SMILES: CCCC[C@H](NC(C)=O)C(=O)N[C@H]1CCC(=O)NC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 510 |
| | MC-1 Ki (average) | 0.04 |
| | MC-1 EC₅₀ (average; cAMP HBL) | 0.008 |
| | MC-1 Eₘₐₓ (average; cAMP HBL) | 91% |
| 9.4 | Ac-Nle-cyclo(Asp-His-D-Phe-Arg-Dap)-Trp-NH₂ (SEQ ID NO: 7) | |
| | 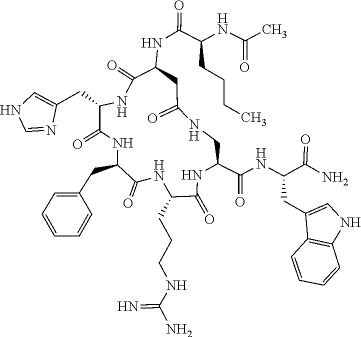 [SMILES: CCCC[C@H](NC(C)=O)C(=O)N[C@H]1CC(=O)NC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 1325 |
| | MC-1 Ki (average) | 2 |
| | MC-1 EC₅₀ (average; cAMP HBL) | 0.195 |
| | MC-1 Eₘₐₓ (average; cAMP HBL) | 88% |
| 9.5 | Ac-Nle-cyclo(Cys-His-D-Phe-Arg-Cys)-Trp-NH₂ (SEQ ID NO: 8) | |
| | 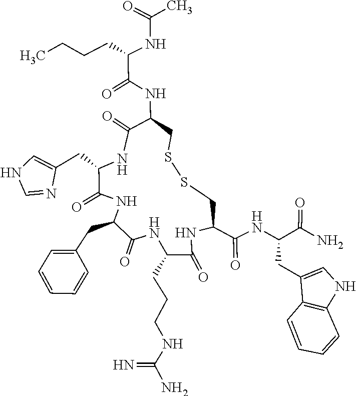 [SMILES: CCCC[C@H](NC(C)=O)C(=O)N[C@H]1CSSC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 540 |
| | MC-1 Ki (average) | 0.35 |
| | MC-1 EC₅₀ (average; cAMP HBL) | 0.025 |
| | MC-1 Eₘₐₓ (average; cAMP HBL) | 87% |
| 9.6 | Ac-Nle-cyclo(Glu-His-D-Phe-Arg-Orn)-Trp-NH₂ (SEQ ID NO: 9) | |
| | 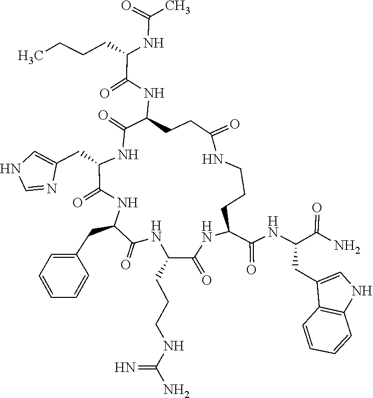 [SMILES: CCCC[C@H](NC(C)=O)C(=O)N[C@H]1CCC(=O)NCCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 295 |
| | MC-1 Ki (average) | 0.07 |
| | MC-1 EC₅₀ (average; cAMP HBL) | 0.014 |
| | MC-1 Eₘₐₓ (average; cAMP HBL) | 91% |
| 9.7 | Ac-Nle-cyclo(Glu-His-D-Phe-Arg-Dab)-D-Trp-NH₂ (SEQ ID NO: 10) | |
| | 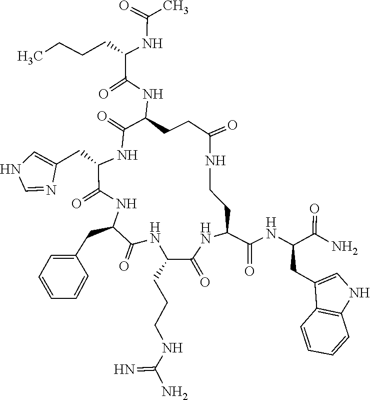 [SMILES: CCCC[C@H](NC(C)=O)C(=O)N[C@H]1CCC(=O)NCC[C@@H](C(=O)N[C@H](Cc2c[nH]c3ccccc23)C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 33 |
| | MC-1 Ki (average) | 0.55 |
| | MC-1 EC₅₀ (average; cAMP HBL) | 0.025 |
| | MC-1 Eₘₐₓ (average; cAMP HBL) | 93% |
| 9.8 | Ac-Nle-cyclo(Glu-His-Phe-Arg-Dab)-Trp-NH₂ (SEQ ID NO: 11) | |
| | 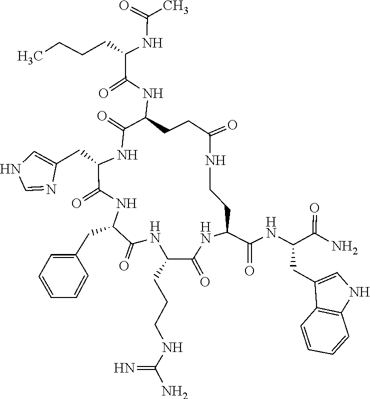 [SMILES: CCCC[C@H](NC(C)=O)C(=O)N[C@H]1CCC(=O)NCC[C@@H](C(=O)N[C@@H](Cc2c[nH]c3ccccc23)C(N)=O)NC(=O)[C@H](CCCNC(=N)N)NC(=O)[C@H](Cc2ccccc2)NC(=O)[C@H](Cc2c[nH]cn2)NC1=O] | |
| | Assay | Result |
| | MC-4 Ki (average) | 10000 |
| | MC-1 Ki (average) | 2 |
| | MC-1 EC₅₀ (average; cAMP HBL) | 0.052 |
| | MC-1 Eₘₐₓ (average; cAMP HBL) | 88% |
| 9.9 | Ac-Nle-cyclo(Glu-His-D-Phe-Arg-Dab)-D-Nal 1-NH₂ (SEQ ID NO: 12) | |
| | 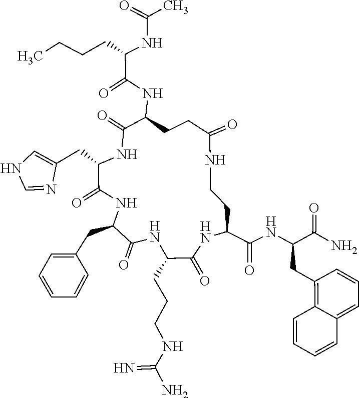 [SMILES: CCCC[C@H](NC(C)=O)C(=O)N[C@H]1CCC(=O)NCC[C@@H](C(=O)N[C@H](Cc2cccc3ccccc23)C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 2 |
| | MC-1 Ki (average) | 0.1 |
| | MC-1 EC₅₀ (average; cAMP HBL) | 0.008 |
| | MC-1 Eₘₐₓ (average; cAMP HBL) | 73% |
| 9.10 | Ac-Nle-cyclo(Glu-His-D-Phe-Arg-Dab)-Nal 2-NH₂ (SEQ ID NO: 13) | |
| | 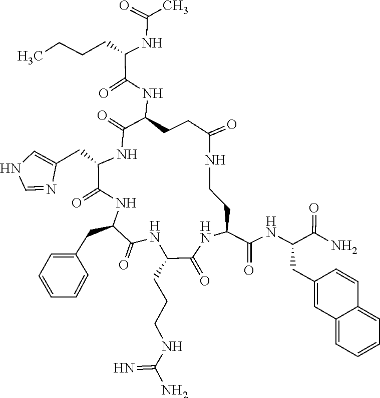 [SMILES: CCCC[C@H](NC(C)=O)C(=O)N[C@H]1CCC(=O)NCC[C@@H](C(=O)N[C@@H](Cc2ccc3ccccc3c2)C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 3 |
| | MC-1 Ki (average) | 0.01 |
| | MC-1 EC₅₀ (average; cAM PHBL) | 0.004 |
| | MC-1 Eₘₐₓ (average; cAMP HBL) | 83% |
| 9.11 | Ac-Nle-cyclo(Glu-His-D-Phe-Arg-Dab)-D-Nal 2-NH₂ (SEQ ID NO: 14) | |
| | 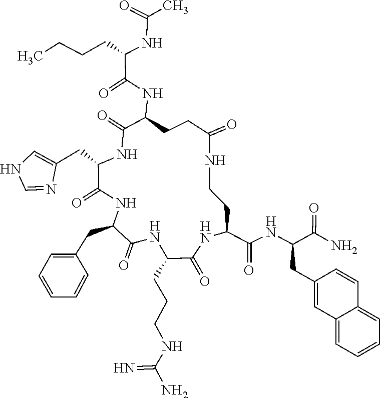 [SMILES: CCCC[C@H](NC(C)=O)C(=O)N[C@H]1CCC(=O)NCC[C@@H](C(=O)N[C@H](Cc2ccc3ccccc3c2)C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 0.095 |
| | MC-1 Ki (average) | 0.02 |
| | MC-1 EC₅₀ (average; cAMP HBL) | 0.005 |
| | MC-1 Eₘₐₓ (average; cAMP HBL) | 69% |
| 9.12 | Ac-D-Phe-cyclo(Glu-His-D-Phe-Arg-Dab)-Trp-NH₂ (SEQ ID NO: 15) | |
| | 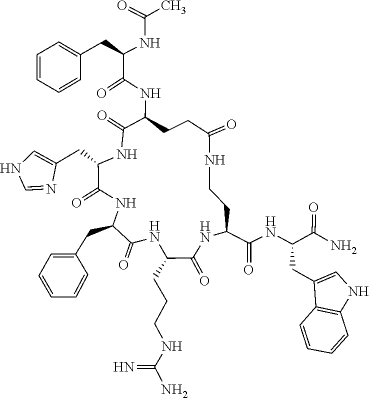 [SMILES: CC(=O)N[C@H](Cc1ccccc1)C(=O)N[C@H]1CCC(=O)NCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 535 |
| | MC-1 Ki (average) | 0.35 |
| | MC-1 EC₅₀ (average; cAMP HBL) | 0.015 |
| | MC-1 Eₘₐₓ (average; cAMP HBL) | 75% |
| 9.13 | Ac-Phe-cyclo(Glu-His-D-Phe-Arg-Dab)-Trp-NH₂ (SEQ ID NO: 16) | |
| | 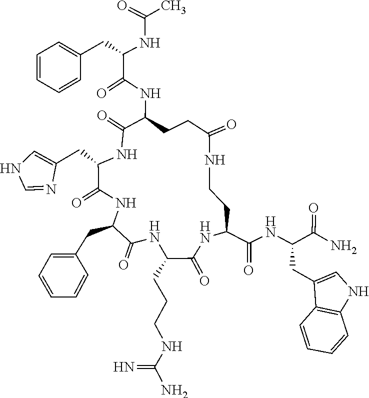 [SMILES: CC(=O)N[C@@H](Cc1ccccc1)C(=O)N[C@H]1CCC(=O)NCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 510 |
| | MC-1 Ki (average) | 0.195 |
| | MC-1 EC₅₀ (average; cAMP HBL) | 0.01 |
| | MC-1 Eₘₐₓ (average; cAMP HBL) | 75% |
| 9.14 | cyclo(Suc-His-Phe-Arg-Dab)-Trp-NH₂ (SEQ ID NO: 17) | |
| | 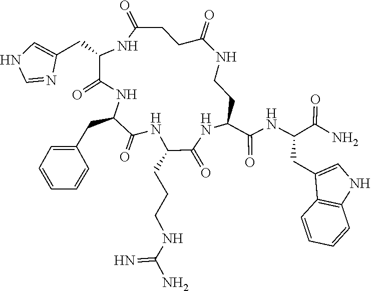 [SMILES: N=C(N)NCCC[C@@H]1NC(=O)[C@@H](Cc2ccccc2)NC(=O)[C@H](Cc2c[nH]cn2)NC(=O)CCC(=O)NCC[C@@H](C(=O)N[C@@H](Cc2c[nH]c3ccccc23)C(N)=O)NC1=O] | |
| | Assay | Result |
| | MC-4 Ki (average) | 10000 |
| | MC-1 Ki (average) | 310 |
| | MC-1 EC₅₀ (average; cAMP HBL) | 31 |
| | MC-1 Eₘₐₓ (average; cAMP HBL) | 80% |
| 9.15 | CH₃—(CH₂)₂—C(═O)-cyclo(Glu-His-D-Phe-Arg-Dab)-Trp-NH₂ (SEQ ID NO: 18) | |
| | 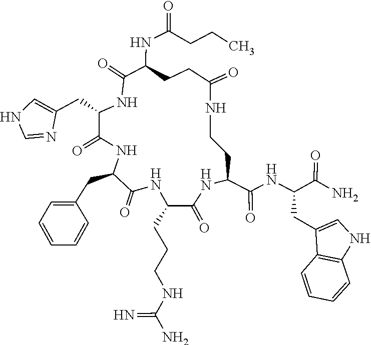 [SMILES: CCCC(=O)N[C@H]1CCC(=O)NCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 890 |
| | MC-1 Ki (average) | 0.65 |
| | MC-1 EC₅₀ (average; cAMP HBL) | 0.012 |
| | MC-1 Eₘₐₓ (average; cAMP HBL) | 91% |
| 9.16 | CH₃—(CH₂)₃—C(═O)-cyclo(Glu-His-D-Phe-Arg-Dab)-Trp-NH₂ (SEQ ID NO: 19) | |
| | 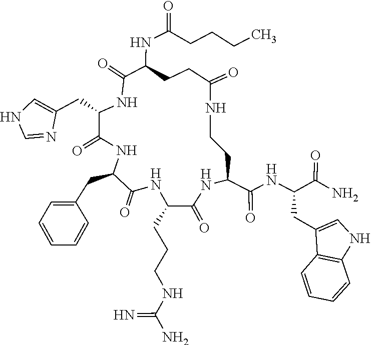 [SMILES: CCCCC(=O)N[C@H]1CCC(=O)NCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 365 |
| | MC-1 Ki (average) | 0.12 |
| | MC-1 EC₅₀ (average; cAMP HBL) | 0.005 |
| | MC-1 Eₘₐₓ (average; cAMP HBL) | 89% |
| 9.17 | CH₃—(CH₂)₄—C(═O)-cyclo(Glu-His-D-Phe-Arg-Dab)-Trp-NH₂ (SEQ ID NO: 20) | |
| | 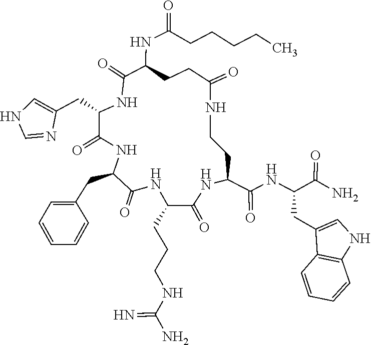 [SMILES: CCCCCC(=O)N[C@H]1CCC(=O)NCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 110 |
| | MC-1 Ki (average) | 0.025 |
| | MC-1 EC₅₀ (average; cAMP HBL) | 0.004 |
| | MC-1 Eₘₐₓ (average; cAMP HBL) | 90% |
| 9.18 | CH₃—(CH₂)₅—C(═O)-cyclo(Glu-His-D-Phe-Arg-Dab)-Trp-NH₂ (SEQ ID NO: 21) | |
| | 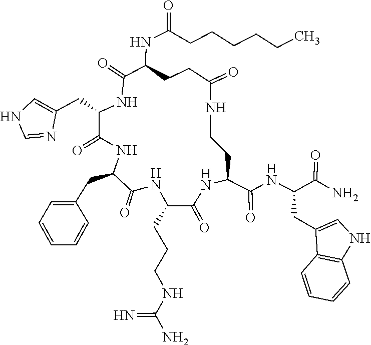 [SMILES: CCCCCCC(=O)N[C@H]1CCC(=O)NCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 100 |
| | MC-1 Ki (average) | 0.015 |
| | MC-1 EC₅₀ (average; cAMP HBL) | 0.003 |
| | MC-1 Eₘₐₓ (average; cAMP HBL) | 87% |
| 9.19 | cyclo-propanoyl-cyclo(Glu-His-D-Phe-Arg-Dab)-Trp-NH₂ (SEQ ID NO: 22) | |
| | 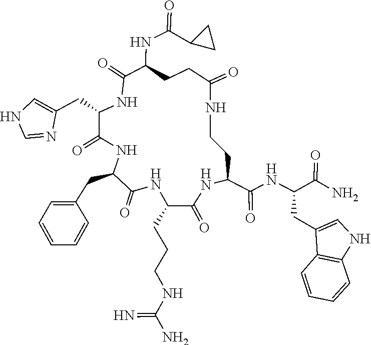 [SMILES: N=C(N)NCCC[C@@H]1NC(=O)[C@@H](Cc2ccccc2)NC(=O)[C@H](Cc2c[nH]cn2)NC(=O)[C@@H](NC(=O)C2CC2)CCC(=O)NCC[C@@H](C(=O)N[C@@H](Cc2c[nH]c3ccccc23)C(N)=O)NC1=O] | |
| | Assay | Result |
| | MC-4 Ki (average) | 640 |
| | MC-1 Ki (average) | 3 |
| | MC-1 EC₅₀ (average; cAMP HBL) | 0.031 |
| | MC-1 Eₘₐₓ (average; cAMP HBL) | 83% |
| 9.20 | cyclo-hexanoyl-cyclo(Glu-His-D-Phe-Arg-Dab)-Trp-NH₂ (SEQ ID NO: 23) | |
| | 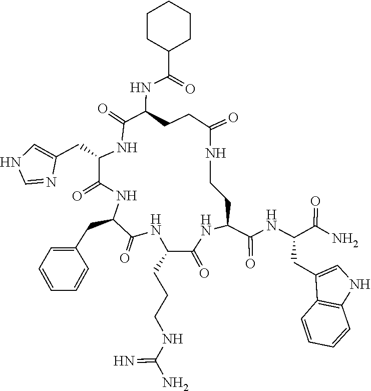 [SMILES: N=C(N)NCCC[C@@H]1NC(=O)[C@@H](Cc2ccccc2)NC(=O)[C@H](Cc2c[nH]cn2)NC(=O)[C@@H](NC(=O)C2CCCCC2)CCC(=O)NCC[C@@H](C(=O)N[C@@H](Cc2c[nH]c3ccccc23)C(N)=O)NC1=O] | |
| | Assay | Result |
| | MC-4 Ki (average) | 165 |
| | MC-1 Ki (average) | 0.025 |
| | MC-1 EC₅₀ (average; cAMP HBL) | 0.004 |
| | MC-1 Eₘₐₓ (average; cAMP HBL) | 79% |
| 9.21 | cyclopentyl acetyl-cyclo(Glu-His-D-Phe-Arg-Dab)-Trp-NH₂ (SEQ ID NO: 24) | |
| | 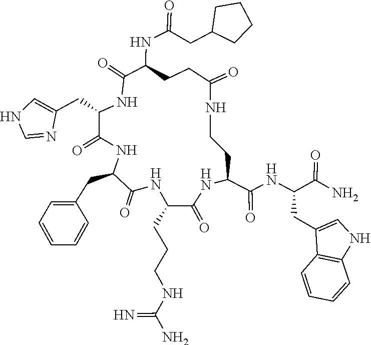 [SMILES: N=C(N)NCCC[C@@H]1NC(=O)[C@@H](Cc2ccccc2)NC(=O)[C@H](Cc2c[nH]cn2)NC(=O)[C@@H](NC(=O)CC2CCCC2)CCC(=O)NCC[C@@H](C(=O)N[C@@H](Cc2c[nH]c3ccccc23)C(N)=O)NC1=O] | |
| | Assay | Result |
| | MC-4 Ki (average) | 93 |
| | MC-1 Ki (average) | 0.01 |
| | MC-1 EC₅₀ (average; cAMP HBL) | 0.004 |
| | MC-1 Eₘₐₓ (average; cAMP HBL) | 83% |
| 9.22 | cyclohexyl acetyl-cyclo(Glu-His-D-Phe-Arg-Dab)-Trp-NH₂ (SEQ ID NO: 25) | |
| | 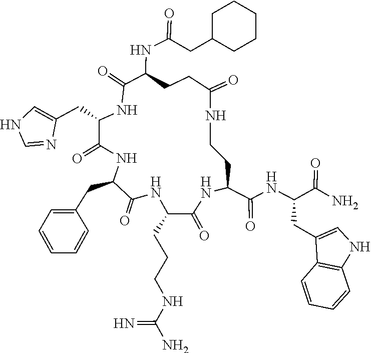 [SMILES: N=C(N)NCCC[C@@H]1NC(=O)[C@@H](Cc2ccccc2)NC(=O)[C@H](Cc2c[nH]cn2)NC(=O)[C@@H](NC(=O)CC2CCCCC2)CCC(=O)NCC[C@@H](C(=O)N[C@@H](Cc2c[nH]c3ccccc23)C(N)=O)NC1=O] | |
| | Assay | Result |
| | MC-4 Ki (average) | 63 |
| | MC-1 Ki (average) | 0.01 |
| | MC-1 EC₅₀ (average; cAMP HBL) | 0.004 |
| | MC-1 Eₘₐₓ (average; cAMP HBL) | 85% |
| 9.23 | phenyl acetyl-cyclo(Glu-His-D-Phe-Arg-Dab)-Trp-NH₂ (SEQ ID NO: 26) | |
| | 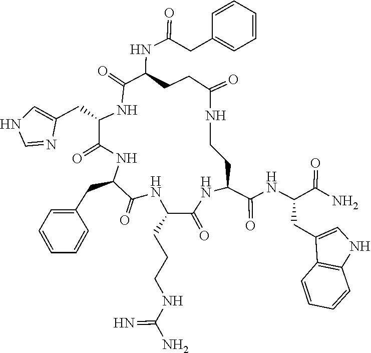 [SMILES: N=C(N)NCCC[C@@H]1NC(=O)[C@@H](Cc2ccccc2)NC(=O)[C@H](Cc2c[nH]cn2)NC(=O)[C@@H](NC(=O)Cc2ccccc2)CCC(=O)NCC[C@@H](C(=O)N[C@@H](Cc2c[nH]c3ccccc23)C(N)=O)NC1=O] | |
| | Assay | Result |
| | MC-4 Ki (average) | 205 |
| | MC-1 Ki (average) | 0.04 |
| | MC-1 EC₅₀ (average; cAMP HBL) | 0.007 |
| | MC-1 Eₘₐₓ (average; cAMP HBL) | 82% |
| 9.24 | phenyl propanoyl-cyclo(Glu-His-D-Phe-Arg-Dab)-Trp-NH₂ (SEQ ID NO: 27) | |
| | 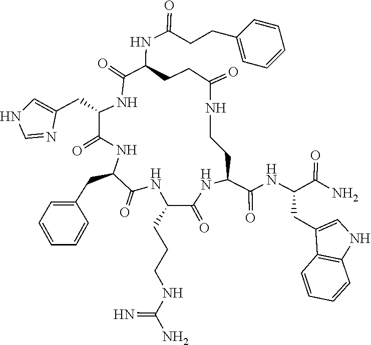 [SMILES: N=C(N)NCCC[C@@H]1NC(=O)[C@@H](Cc2ccccc2)NC(=O)[C@H](Cc2c[nH]cn2)NC(=O)[C@@H](NC(=O)CCc2ccccc2)CCC(=O)NCC[C@@H](C(=O)N[C@@H](Cc2c[nH]c3ccccc23)C(N)=O)NC1=O] | |
| | Assay | Result |
| | MC-4 Ki (average) | 285 |
| | MC-1 Ki (average) | 0.03 |
| | MC-1 EC₅₀ (average; cAMP HBL) | 0.006 |
| | MC-1 Eₘₐₓ (average; cAMP HBL) | 83% |
| 9.25 | Ac-Nle-cyclo(Glu-His-D-Phe-Arg-Dab)-Ala-NH₂ (SEQ ID NO: 28) | |
| | 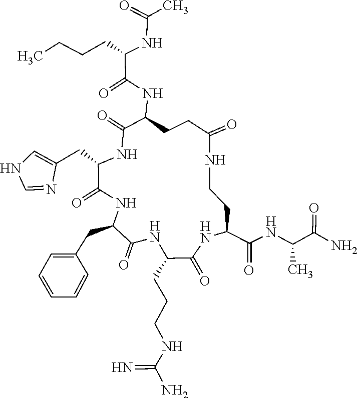 [SMILES: CCCC[C@H](NC(C)=O)C(=O)N[C@H]1CCC(=O)NCC[C@@H](C(=O)N[C@@H](C)C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 7475 |
| | MC-1 Ki (average) | 2 |
| | MC-1 EC₅₀ (average; cAMP HBL) | 0.08 |
| | MC-1 Eₘₐₓ (average; cAMP HBL) | 97% |
| 9.26 | Ac-Nle-cyclo(Glu-His-D-Phe-Arg-Dap)-Trp-OH (SEQ ID NO: 29) | |
| | 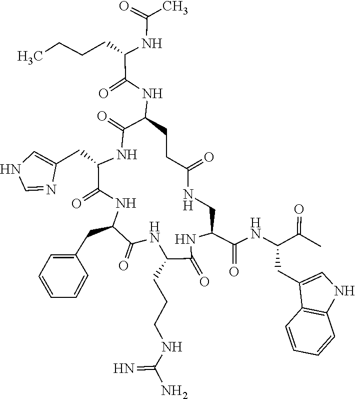 [SMILES: CCCC[C@H](NC(C)=O)C(=O)N[C@H]1CCC(=O)NC[C@@H](C(=O)N[C@@H](Cc2c[nH]c3ccccc23)C(C)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 4700 |
| | MC-1 Ki (average) | 1 |
| | MC-1 EC₅₀ (average; cAMP HBL) | 0.107 |
| | MC-1 Eₘₐₓ (average; cAMP HBL) | 81% |
| 9.27 | Ac-Nle-cyclo(Dab-His-D-Phe-Arg-Dab)-Trp-NH₂ (SEQ ID NO: 30) | |
| | 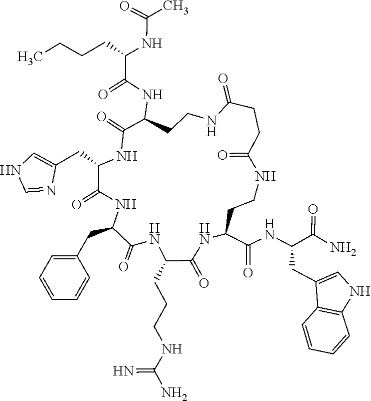 [SMILES: CCCC[C@H](NC(C)=O)C(=O)N[C@H]1CCNC(=O)CCC(=O)NCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 380 |
| | MC-1 Ki (average) | 0.015 |
| | MC-1 EC₅₀ (average; cAMP HBL) | 0.009 |
| | MC-1 Eₘₐₓ (average; cAMP HBL) | 92% |
| 9.28 | Ac-Nle-cyclo(Dap-His-D-Phe-Arg-Dap)-Trp-NH₂ (SEQ ID NO: 31) | |
| | 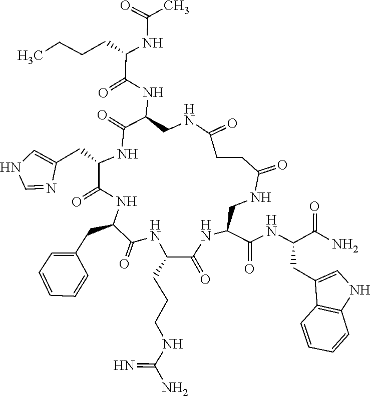 [SMILES: CCCC[C@H](NC(C)=O)C(=O)N[C@H]1CNC(=O)CCC(=O)NC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 680 |
| | MC-1 Ki (average) | 0.03 |
| | MC-1 EC₅₀ (average; cAMP HBL) | 0.007 |
| | MC-1 Eₘₐₓ (average; cAMP HBL) | 70% |
| 9.29 | Ac-Nle-cyclo(Dab-His-D-Phe-Arg-Dap)-Trp-NH₂ (SEQ ID NO: 32) | |
| | 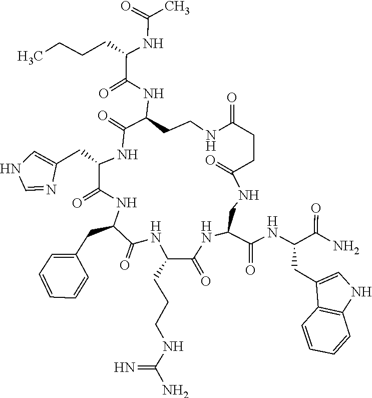 [SMILES: CCCC[C@H](NC(C)=O)C(=O)N[C@H]1CCNC(=O)CCC(=O)NC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 325 |
| | MC-1 Ki (average) | 0.025 |
| | MC-1 EC₅₀ (average; cAMP HBL) | 0.006 |
| | MC-1 Eₘₐₓ (average; cAMP HBL) | 78% |
| 9.30 | Ac-Nle-cyclo(Glu-His-D-Phe-Arg-Dab)-Nal 1-NH₂ (SEQ ID NO: 33) | |
| | 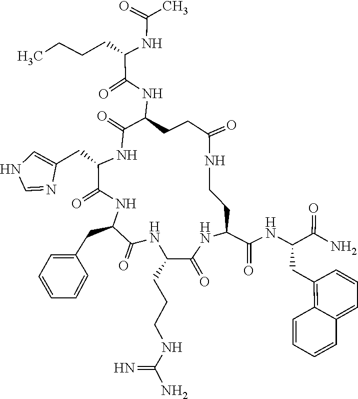 [SMILES: CCCC[C@H](NC(C)=O)C(=O)N[C@H]1CCC(=O)NCC[C@@H](C(=O)N[C@@H](Cc2cccc3ccccc23)C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 0.75 |
| | MC-1 Ki (average) | 0.005 |
| | MC-1 EC₅₀ (average; cAMP HBL) | 0.002 |
| | MC-1 Eₘₐₓ (average; cAMP HBL) | 72% |
| 9.31 | Ac-Nle-cyclo(Glu-His-D-Phe-Arg-Dab)-Trp-OH (SEQ ID NO: 34) | |
| | 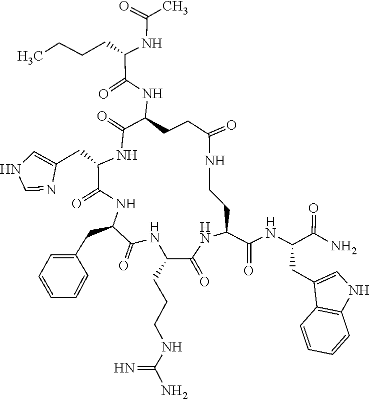 [SMILES: CCCC[C@H](NC(C)=O)C(=O)N[C@H]1CCC(=O)NCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 10000 |
| | MC-1 Ki (average) | 2 |
| | MC-1 EC₅₀ (average; cAMP HBL) | 0.095 |
| | MC-1 Eₘₐₓ (average; cAMP HBL) | 79% |
| 9.32 | Ac-Nle-cyclo(Glu-His-D-Phe-Arg-Dab)-NH₂ (SEQ ID NO: 35) | |
| | 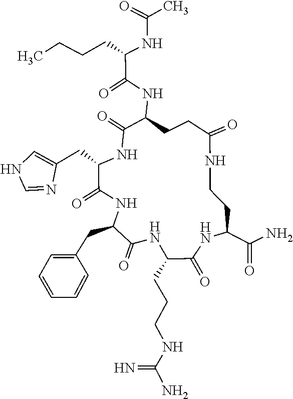 [SMILES: CCCC[C@H](NC(C)=O)C(=O)N[C@H]1CCC(=O)NCC[C@@H](C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 10000 |
| | MC-1 Ki (average) | 7 |
| | MC-1 EC₅₀ (average; cAMP HBL) | 0.32 |
| | MC-1 Eₘₐₓ (average; cAMP HBL) | 85% |
| 9.33 | Ac-Nle-cyclo(Glu-His-D-Phe-Ala-Dab)-NH₂ (SEQ ID NO: 36) | |
| | 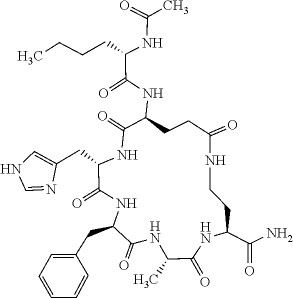 [SMILES: CCCC[C@H](NC(C)=O)C(=O)N[C@H]1CCC(=O)NCC[C@@H](C(N)=O)NC(=O)[C@H](C)NC(=O)[C@@H](Cc2ccccc2)NC(=O)[C@H](Cc2c[nH]cn2)NC1=O] | |
| | Assay | Result |
| | MC-4 Ki (average) | 10000 |
| | MC-1 Ki (average) | 645 |
| | MC-1 EC₅₀ (average; cAMP HBL) | 44 |
| | MC-1 Eₘₐₓ (average; cAMP HBL) | 73% |
| 9.34 | Ac-Nle-cyclo(Glu-His-D-Phe-Gly-Dab)-Trp-NH₂ (SEQ ID NO: 37) | |
| | 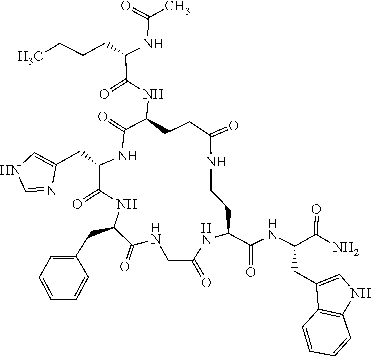 [SMILES: CCCC[C@H](NC(C)=O)C(=O)N[C@H]1CCC(=O)NCC[C@@H](C(=O)N[C@@H](Cc2c[nH]c3ccccc23)C(N)=O)NC(=O)CNC(=O)[C@@H](Cc2ccccc2)NC(=O)[C@H](Cc2c[nH]cn2)NC1=O] | |
| | Assay | Result |
| | MC-4 Ki (average) | 10000 |
| | MC-1 Ki (average) | 9 |
| | MC-1 EC₅₀ (average; cAMP HBL) | 4 |
| | MC-1 Eₘₐₓ (average; cAMP HBL) | 59% |
| 9.35 | Ac-Nle-cyclo(Glu-His-D-Phe-Ala-Dab)-Trp-NH₂ (SEQ ID NO: 38) | |
| | 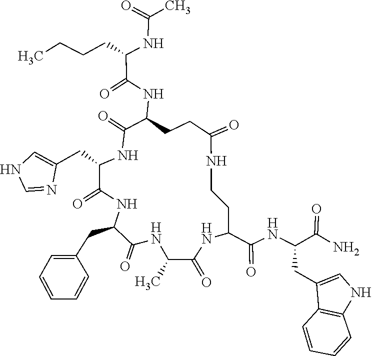 [SMILES: CCCC[C@H](NC(C)=O)C(=O)N[C@H]1CCC(=O)NCCC(C(=O)N[C@@H](Cc2c[nH]c3ccccc23)C(N)=O)NC(=O)[C@H](C)NC(=O)[C@@H](Cc2ccccc2)NC(=O)[C@H](Cc2c[nH]cn2)NC1=O] | |
| | Assay | Result |
| | MC-4 Ki (average) | 10000 |
| | MC-1 Ki (average) | 0.8 |
| | MC-1 EC₅₀ (average; cAMP HBL) | 0.115 |
| | MC-1 Eₘₐₓ (average; cAMP HBL) | 72% |
| 9.36 | Ac-Nle-cyclo(Glu-Ala-D-Phe-Arg-Dab)-Trp-NH₂ (SEQ ID NO: 39) | |
| | 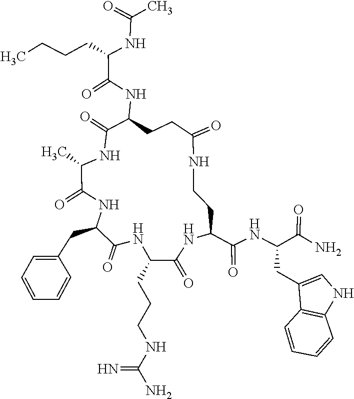 [SMILES: CCCC[C@H](NC(C)=O)C(=O)N[C@H]1CCC(=O)NCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](C)NC1=O] | |
| | Assay | Result |
| | MC-4 Ki (average) | 455 |
| | MC-1 Ki (average) | 4 |
| | MC-1 EC₅₀ (average; cAMP HBL) | 0.21 |
| | MC-1 Eₘₐₓ (average; cAMP HBL) | 86% |
| 9.37 | Ac-Nle-cyclo(Glu-Arg-D-Phe-Arg-Dab)-Trp-NH₂ (SEQ ID NO: 40) | |
| | 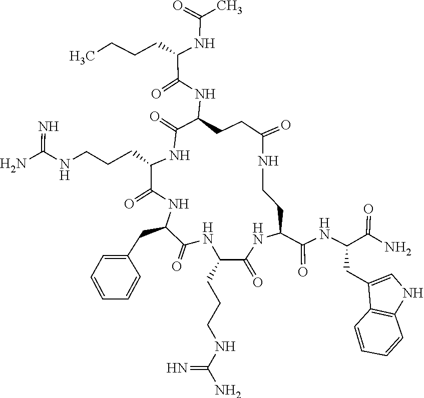 [SMILES: CCCC[C@H](NC(C)=O)C(=O)N[C@H]1CCC(=O)NCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](CCCNC(=N)N)NC1=O] | |
| | Assay | Result |
| | MC-4 Ki (average) | 20 |
| | MC-1 Ki (average) | 0.014 |
| | MC-1 EC₅₀ (average; cAMP HBL) | 0.003 |
| | MC-1 Eₘₐₓ (average; cAMP HBL) | 100% |
| 9.38 | Ac-Nle-cyclo(Glu-Cit-D-Phe-Arg-Dab)-Trp-NH₂ (SEQ ID NO: 41) | |
| | 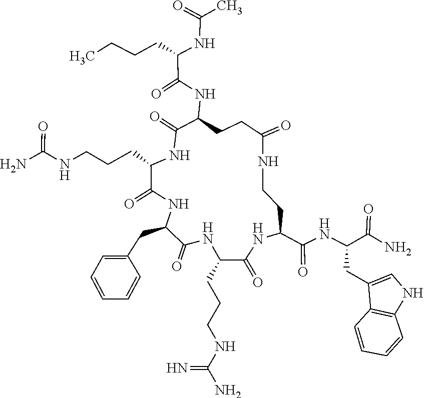 [SMILES: CCCC[C@H](NC(C)=O)C(=O)N[C@H]1CCC(=O)NCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](CCCNC(N)=O)NC1=O] | |
| | Assay | Result |
| | MC-4 Ki (average) | 98 |
| | MC-1 Ki (average) | 0.45 |
| | MC-1 EC₅₀ (average; cAMP HBL) | 0.065 |
| | MC-1 Eₘₐₓ (average; cAMP HBL) | 97% |
| 9.39 | Ac-Nle-cyclo(Glu-His-D-Phe-Arg-Dab)-Lys-NH₂ (SEQ ID NO: 42) | |
| | 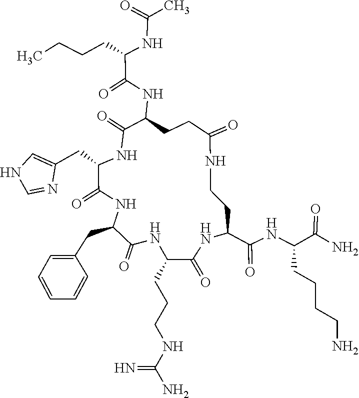 [SMILES: CCCC[C@H](NC(C)=O)C(=O)N[C@H]1CCC(=O)NCC[C@@H](C(=O)N[C@@H](CCCCN)C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 7375 |
| | MC-1 Ki (average) | 2 |
| | MC-1 EC₅₀ (average; cAMP HBL) | 0.175 |
| | MC-1 Eₘₐₓ (average; cAMP HBL) | 91% |
| 9.40 | Ac-Nle-cyclo(Glu-Lys-D-Phe-Arg-Dab)-Trp-NH₂ (SEQ ID NO: 43) | |
| | 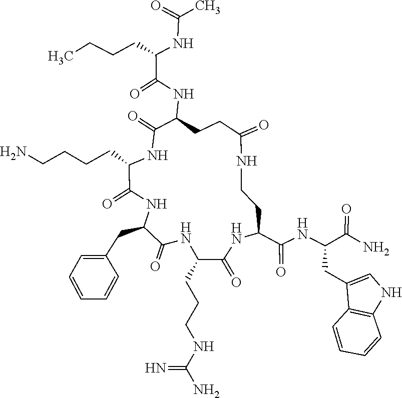 [SMILES: CCCC[C@H](NC(C)=O)C(=O)N[C@H]1CCC(=O)NCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](CCCCN)NC1=O] | |
| | Assay | Result |
| | MC-4 Ki (average) | 95 |
| | MC-1 Ki (average) | 0.04 |
| | MC-1 EC₅₀ (average; cAMP HBL) | 0.006 |
| | MC-1 Eₘₐₓ (average; cAMP HBL) | 108% |
| 9.41 | Ac-Nle-cyclo(Glu-Dab-D-Phe-Arg-Dab)-Trp-NH₂ (SEQ ID NO: 44) | |
| | 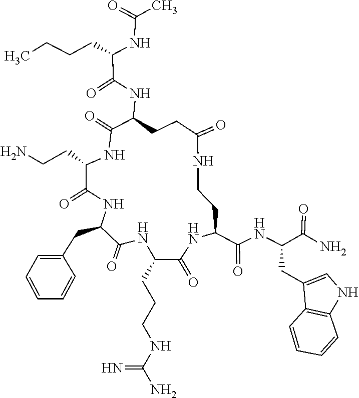 [SMILES: CCCC[C@H](NC(C)=O)C(=O)N[C@H]1CCC(=O)NCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](CCN)NC1=O] | |
| | Assay | Result |
| | MC-4 Ki (average) | 98 |
| | MC-1 Ki (average) | 0.05 |
| | MC-1 EC₅₀ (average; cAMP HBL) | 0.008 |
| | MC-1 Eₘₐₓ (average; cAMP HBL) | 108% |
| 9.42 | Ac-Nle-cyclo(Glu-Orn-D-Phe-Arg-Dab)-Trp-NH₂ (SEQ ID NO: 45) | |
| | 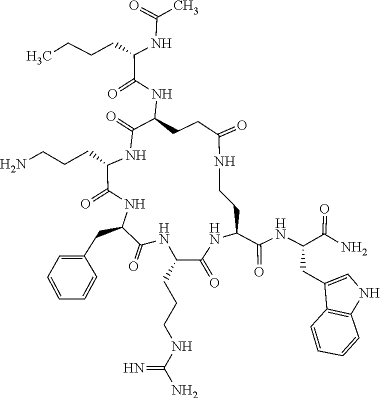 [SMILES: CCCC[C@H](NC(C)=O)C(=O)N[C@H]1CCC(=O)NCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](CCCN)NC1=O] | |
| | Assay | Result |
| | MC-4 Ki (average) | 45 |
| | MC-1 Ki (average) | 0.015 |
| | MC-1 EC5₀ (average; cAMP HBL) | 0.002 |
| | MC-1 Eₘₐₓ (average; cAMP HBL) | 109% |
| 9.43 | Ac-Nle-cyclo(Dap-His-D-Phe-Arg-Dab)-Trp-NH₂ (SEQ ID NO: 46) | |
| | 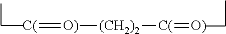 | |
| | 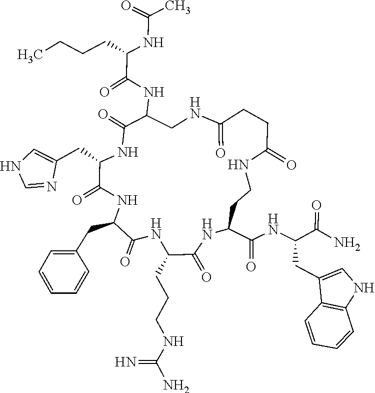 [SMILES: CCCC[C@H](NC(C)=O)C(=O)NC1CNC(=O)CCC(=O)NCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](Cc2c[nH]cn2)NC1=O] | |
| | Assay | Result |
| | MC-4 Ki (average) | 860 |
| | MC-1 Ki (average) | 0.065 |
| | MC-1 EC₅₀ (average; cAMP HBL) | 0.016 |
| | MC-1 Eₘₐₓ (average; cAMP HBL) | 85% |
| 9.44 | Ac-Nle-cyclo(Glu-His-D-Phe-Arg-Dab)-NH₂ (SEQ ID NO: 47) | |
| | 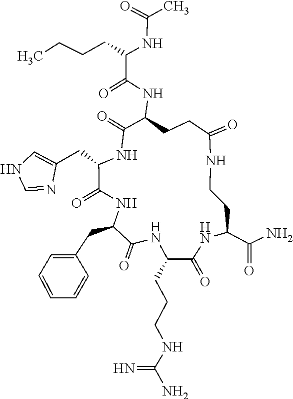 | |
| | Assay | Result |
| | MC-4 Ki (average) | 10000 |
| | MC-1 Ki (average) | 7 |
| | MC-1 EC₅₀ (average; cAM PHBL) | 0.32 |
| | MC-1 Eₘₐₓ (average; cAMP HBL) | 85% |
| 9.45 | Ac-Nle-cyclo(Glu-His-D-Phe-Ala-Dab)-NH₂ (SEQ ID NO: 48) | |
| | 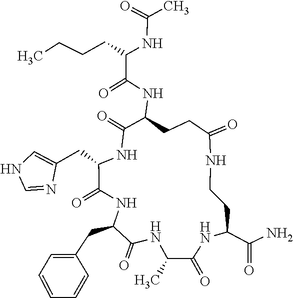 | |
| | Assay | Result |
| | MC-4 Ki (average) | 10000 |
| | MC-1 Ki (average) | 645 |
| | MC-1 EC₅₀ (average; cAMPHBL) | 44 |
| | MC-1 Eₘₐₓ (average; cAMP HBL) | 73% |
| 9.46 | Ac-Nle-cyclo(Glu-His-D-Phe-Dab)-NH₂ (SEQ ID NO: 49) | |
| | 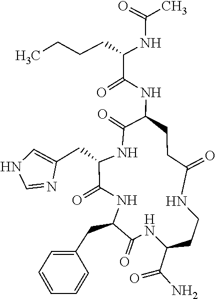 [SMILES: CCCC[C@H](NC(C)=O)C(=O)N[C@H]1CCC(=O)NCC[C@@H](C(N)=O)NC(=O)[C@@H](Cc2ccccc2)NC(=O)[C@H](Cc2c[nH]cn2)NC1=O] | |
| | Assay | Result |
| | MC-4 Ki (average) | 10000 |
| | MC-1 Ki (average) | 10000 |
| | MC-1 EC₅₀ (average; cAMP HBL) | NA |
| | MC-1 Eₘₐₓ (average; cAMP HBL) | 51% |
| 9.47 | Ac-Nle-cyclo(Glu-His-D-Phe-Gly-Dab)-Trp-NH₂ (SEQ ID NO: 50) | |
| | 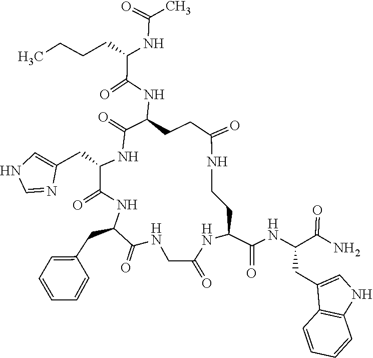 [SMILES: CCCC[C@H](NC(C)=O)C(=O)N[C@H]1CCC(=O)NCC[C@@H](C(=O)N[C@@H](Cc2c[nH]c3ccccc23)C(N)=O)NC(=O)CNC(=O)[C@@H](Cc2ccccc2)NC(=O)[C@H](Cc2c[nH]cn2)NC1=O] | |
| | Assay | Result |
| | MC-4 Ki (average) | 10000 |
| | MC-1 Ki (average) | 9 |
| | MC-1 EC₅₀ (average; cAMP HBL) | 4 |
| | MC-1 Eₘₐₓ (average; cAMP HBL) | 59% |
| 9.48 | Ac-Nle-cyclo(Glu-His-D-Phe-Ala-Dab)-Trp-NH₂ (SEQ ID NO: 51) | |
| | 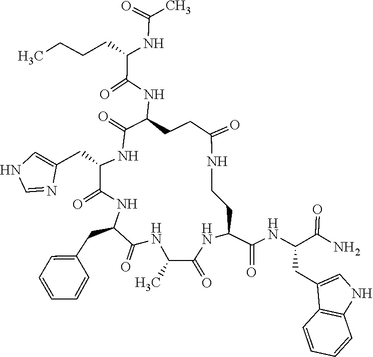 [SMILES: CCCC[C@H](NC(C)=O)C(=O)N[C@H]1CCC(=O)NCC[C@@H](C(=O)N[C@@H](Cc2c[nH]c3ccccc23)C(N)=O)NC(=O)[C@H](C)NC(=O)[C@@H](Cc2ccccc2)NC(=O)[C@H](Cc2c[nH]cn2)NC1=O] | |
| | Assay | Result |
| | MC-4 Ki (average) | 10000 |
| | MC-1 Ki (average) | 0.8 |
| | MC-1 EC₅₀ (average; cAMP HBL) | 0.115 |
| | MC-1 Eₘₐₓ (average; cAMP HBL) | 72% |
| 9.49 | Ac-Nle-cyclo(Glu-Ala-D-Phe-Arg-Dab)-Trp-NH₂ (SEQ ID NO: 52) | |
| | 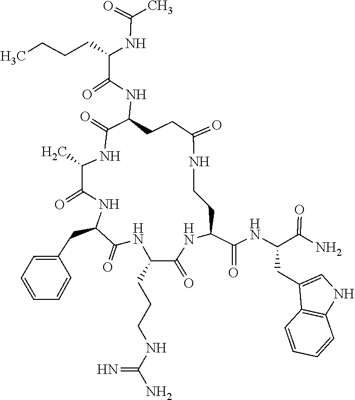 [SMILES: CCCC[C@H](NC(C)=O)C(=O)N[C@H]1CCC(=O)NCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](C)NC1=O] | |
| | Assay | Result |
| | MC-4 Ki (average) | 455 |
| | MC-1 Ki (average) | 4 |
| | MC-1 EC₅₀ (average; cAMP HBL) | 0.21 |
| | MC-1 Eₘₐₓ (average; cAMP HBL) | 86% |
| 9.50 | Ac-Nle-cyclo(Glu-Arg-D-Phe-Arg-Dab)-Trp-NH₂ (SEQ ID NO: 53) | |
| | 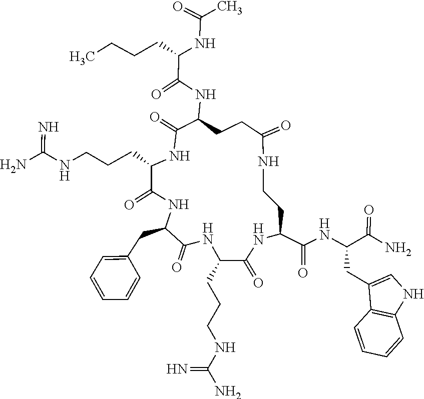 [SMILES: CCCC[C@H](NC(C)=O)C(=O)N[C@H]1CCC(=O)NCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](CCCNC(=N)N)NC1=O] | |
| | Assay | Result |
| | MC-4 Ki (average) | 20 |
| | MC-1 Ki (average) | 0.014 |
| | MC-1 EC₅₀ (average; cAMP HBL) | 0.003 |
| | MC-1 Eₘₐₓ (average; cAMP HBL) | 100% |
| 9.51 | Ac-Nle-cyclo(Glu-Cit-D-Phe-Arg-Dab)-Trp-NH₂ (SEQ ID NO: 54) | |
| | 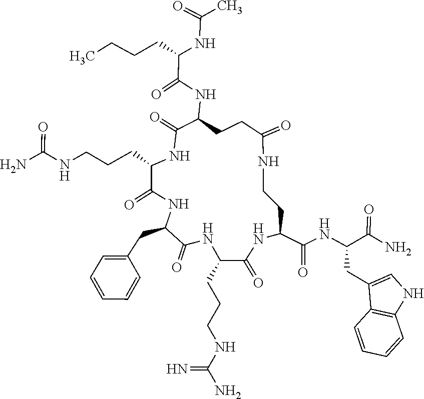 [SMILES: CCCC[C@H](NC(C)=O)C(=O)N[C@H]1CCC(=O)NCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](CCCNC(N)=O)NC1=O] | |
| | Assay | Result |
| | MC-4 Ki (average) | 98 |
| | MC-1 Ki (average) | 0.45 |
| | MC-1 EC₅₀ (average; cAMP HBL) | 0.065 |
| | MC-1 Eₘₐₓ (average; cAMP HBL) | 97% |
| 9.52 | Ac-Nle-cyclo(Glu-His-D-Phe-Arg-Dab)-Lys-NH₂ (SEQ ID NO: 55) | |
| | 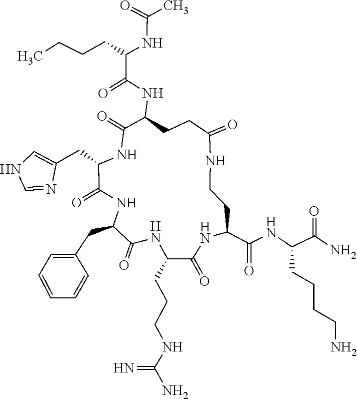 | |
| | Assay | Result |
| | MC-4 Ki (average) | 7375 |
| | MC-1 Ki (average) | 2 |
| | MC-1 EC₅₀ (average; cAMP HBL) | 0.175 |
| | MC-1 Eₘₐₓ (average; cAMP HBL) | 91% |
| 9.53 | Ac-Nle-cyclo(Glu-Lys-D-Phe-Arg-Dab)-Trp-NH₂ (SEQ ID NO: 56) | |
| | 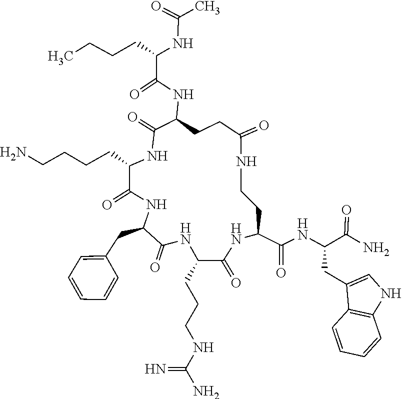 [SMILES: CCCC[C@H](NC(C)=O)C(=O)N[C@H]1CCC(=O)NCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](CCCCN)NC1=O] | |
| | Assay | Result |
| | MC-4 Ki (average) | 95 |
| | MC-1 Ki (average) | 0.04 |
| | MC-1 EC₅₀ (average; cAMP HBL) | 0.006 |
| | MC-1 Eₘₐₓ (average; cAMP HBL) | 108% |
| 9.54 | Ac-Nle-cyclo(Glu-Dab-D-Phe-Arg-Dab)-Trp-NH₂ (SEQ ID NO: 57) | |
| | 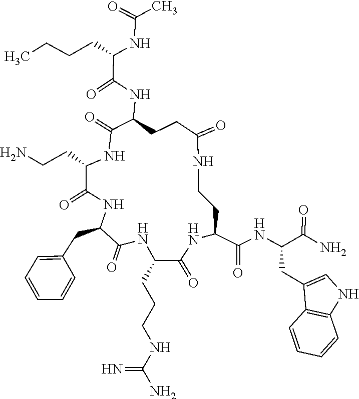 [SMILES: CCCC[C@H](NC(C)=O)C(=O)N[C@H]1CCC(=O)NCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](CCN)NC1=O] | |
| | Assay | Result |
| | MC-4 Ki (average) | 98 |
| | MC-1 Ki (average) | 0.05 |
| | MC-1 EC₅₀ (average; cAMP HBL) | 0.008 |
| | MC-1 Eₘₐₓ (average; cAMP HBL) | 108% |
| 9.55 | Ac-Nle-cyclo(Glu-Orn-D-Phe-Arg-Dab)-Trp-NH₂ (SEQ ID NO: 58) | |
| | 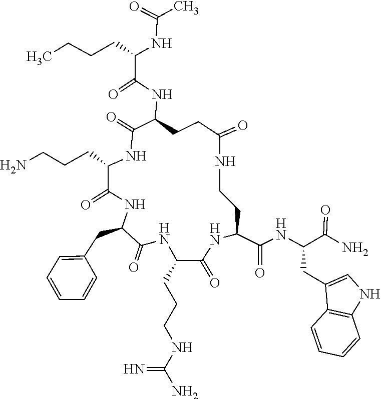 [SMILES: CCCC[C@H](NC(C)=O)C(=O)N[C@H]1CCC(=O)NCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H](CCCN)NC1=O] | |
| | Assay | Result |
| | MC-4 Ki (average) | 45 |
| | MC-1 Ki (average) | 0.015 |
| | MC-1 EC₅₀ (average; cAMP HBL) | 0.002 |
| | MC-1 Eₘₐₓ (average; cAMP HBL) | 109% |
| 9.56 | Ac-Nle-cyclo(Glu-Orn-D-Phe-Arg-Dab)-NH₂ (SEQ ID NO: 59) | |
| | 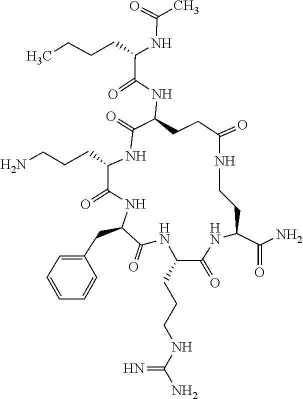 [SMILES: CCCC[C@H](NC(C)=O)C(=O)N[C@H]1CCC(=O)NCC[C@@H](C(N)=O)NC(=O)[C@H](CCCNC(=N)N)NC(=O)[C@@H](Cc2ccccc2)NC(=O)[C@H](CCCN)NC1=O] | |
| | Assay | Result |
| | MC-4 Ki (average) | 3625 |
| | MC-1 Ki (average) | 4 |
| | MC-1 EC₅₀ (average; cAMP HBL) | 0.5 |
| | MC-1 Eₘₐₓ (average; cAMP HBL) | 102% |
| 9.57 | Ac-Nle-cyclo(Glu-Orn-D-Phe(2-Cl)-Arg-Dab)-NH₂ (SEQ ID NO: 60) | |
| | 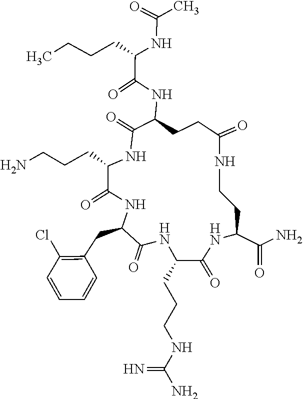 [SMILES: CCCC[C@H](NC(C)=O)C(=O)N[C@H]1CCC(=O)NCC[C@@H](C(N)=O)NC(=O)[C@H](CCCNC(=N)N)NC(=O)[C@@H](Cc2ccccc2Cl)NC(=O)[C@H](CCCN)NC1=O] | |
| | Assay | Result |
| | MC-4 Ki (average) | 10000 |
| | MC-1 Ki (average) | 30 |
| | MC-1 EC₅₀ (average; cAMP HBL) | 1 |
| | MC-1 Eₘₐₓ (average; cAMP HBL) | 88% |
| 9.58 | Ac-Nle-cyclo(Glu-Orn-D-Phe(3-Cl)-Arg-Dab)-NH₂ (SEQ ID NO: 61) | |
| | 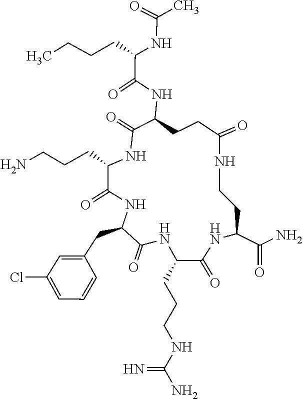 [SMILES: CCCC[C@H](NC(C)=O)C(=O)N[C@H]1CCC(=O)NCC[C@@H](C(N)=O)NC(=O)[C@H](CCCNC(=N)N)NC(=O)[C@@H](Cc2cccc(Cl)c2)NC(=O)[C@H](CCCN)NC1=O] | |
| | Assay | Result |
| | MC-4 Ki (average) | 10000 |
| | MC-1 Ki (average) | 35 |
| | MC-1 EC₅₀ (average; cAMP HBL) | 2 |
| | MC-1 Eₘₐₓ (average; cAMP HBL) | 86% |
| 9.59 | Ac-Nle-cyclo(Glu-Orn-D-Phe(4-Cl)-Arg-Dab)-NH₂ (SEQ ID NO: 62) | |
| | 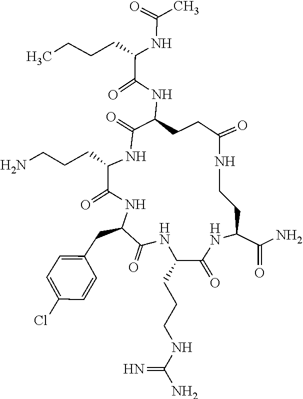 [SMILES: CCCC[C@H](NC(C)=O)C(=O)N[C@H]1CCC(=O)NCC[C@@H](C(N)=O)NC(=O)[C@H](CCCNC(=N)N)NC(=O)[C@@H](Cc2ccc(Cl)cc2)NC(=O)[C@H](CCCN)NC1=O] | |
| | Assay | Result |
| | MC-4 Ki (average) | 1235 |
| | MC-1 Ki (average) | 2 |
| | MC-1 EC₅₀ (average; cAMP HBL) | 0.1 |
| | MC-1 Eₘₐₓ (average; cAMP HBL) | 100% |
| 9.60 | Ac-Nle-cyclo(Glu-Orn-D-Phe(2-F)-Arg-Dab)-NH₂ (SEQ ID NO: 63) | |
| | 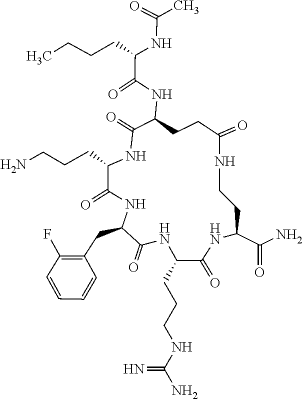 [SMILES: CCCC[C@H](NC(C)=O)C(=O)N[C@H]1CCC(=O)NCC[C@@H](C(N)=O)NC(=O)[C@H](CCCNC(=N)N)NC(=O)[C@@H](Cc2ccccc2F)NC(=O)[C@H](CCCN)NC1=O] | |
| | Assay | Result |
| | MC-4 Ki (average) | 10000 |
| | MC-1 Ki (average) | 18 |
| | MC-1 EC₅₀ (average; cAMP HBL) | 0.8 |
| | MC-1 Eₘₐₓ (average; cAMP HBL) | 95% |
| 9.61 | Ac-Nle-cyclo(Glu-Orn-D-Phe(4-F)-Arg-Dab)-NH₂ (SEQ ID NO: 64) | |
| | 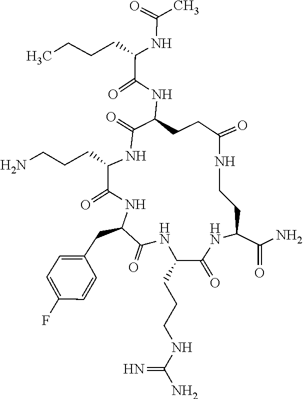 [SMILES: CCCC[C@H](NC(C)=O)C(=O)N[C@H]1CCC(=O)NCC[C@@H](C(N)=O)NC(=O)[C@H](CCCNC(=N)N)NC(=O)[C@@H](Cc2ccc(F)cc2)NC(=O)[C@H](CCCN)NC1=O] | |
| | Assay | Result |
| | MC-4 Ki (average) | 10000 |
| | MC-1 Ki (average) | 20 |
| | MC-1 EC₅₀ (average; cAMP HBL) | 1 |
| | MC-1 Eₘₐₓ (average; cAMP HBL) | 102% |
| 9.62 | Ac-Nle-cyclo(Glu-Orn-D-Phe(3,4-F)-Arg-Dab)-NH₂ (SEQ ID NO: 65) | |
| | 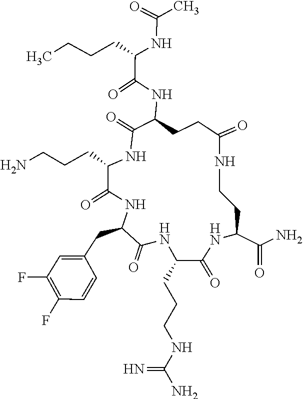 [SMILES: CCCC[C@H](NC(C)=O)C(=O)N[C@H]1CCC(=O)NCC[C@@H](C(N)=O)NC(=O)[C@H](CCCNC(=N)N)NC(=O)[C@@H](Cc2ccc(F)c(F)c2)NC(=O)[C@H](CCCN)NC1=O] | |
| | Assay | Result |
| | MC-4 Ki (average) | 10000 |
| | MC-1 Ki (average) | 40 |
| | MC-1 EC₅₀ (average; cAMP HBL) | 1 |
| | MC-1 Eₘₐₓ (average; cAMP HBL) | 97% |
| 9.63 | Ac-Nle-cyclo(Glu-Orn-D-Phe(4-Me)-Arg-Dab)-NH₂ (SEQ ID NO: 66) | |
| | 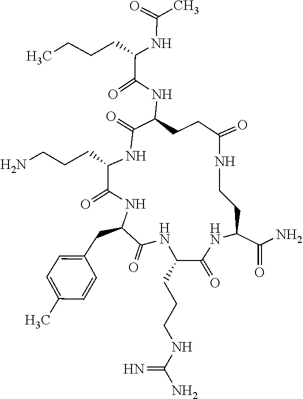 [SMILES: CCCC[C@H](NC(C)=O)C(=O)N[C@H]1CCC(=O)NCC[C@@H](C(N)=O)NC(=O)[C@H](CCCNC(=N)N)NC(=O)[C@@H](Cc2ccc(C)cc2)NC(=O)[C@H](CCCN)NC1=O] | |
| | Assay | Result |
| | MC-4 Ki (average) | 10000 |
| | MC-1 Ki (average) | 3 |
| | MC-1 EC₅₀ (average; cAMP HBL) | 0.076 |
| | MC-1 Eₘₐₓ (average; cAMP HBL) | 96% |
| 9.64 | Ac-Nle-cyclo(Glu-Orn-D-Phe(4-OMe)-Arg-Dab)-NH₂ (SEQ ID NO: 67) | |
| | 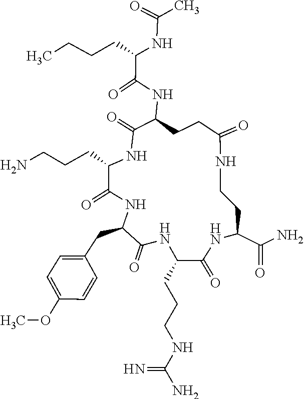 [SMILES: CCCC[C@H](NC(C)=O)C(=O)N[C@H]1CCC(=O)NCC[C@@H](C(N)=O)NC(=O)[C@H](CCCNC(=N)N)NC(=O)[C@@H](Cc2ccc(OC)cc2)NC(=O)[C@H](CCCN)NC1=O] | |
| | Assay | Result |
| | MC-4 Ki (average) | 10000 |
| | MC-1 Ki (average) | 2 |
| | MC-1 EC₅₀ (average; cAMP HBL) | 0.12 |
| | MC-1 Eₘₐₓ (average; cAMP HBL) | 99% |
| 9.65 | Ac-Nle-cyclo(Glu-Pro-D-Phe-Arg-Dab)-NH₂ (SEQ ID NO: 68) | |
| | 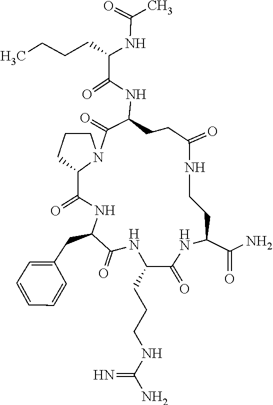 [SMILES: CCCC[C@H](NC(C)=O)C(=O)N[C@H]1CCC(=O)NCC[C@@H](C(N)=O)NC(=O)[C@H](CCCNC(=N)N)NC(=O)[C@@H](Cc2ccccc2)NC(=O)[C@@H]2CCCN2C1=O] | |
| | Assay | Result |
| | MC-4 Ki (average) | 10000 |
| | MC-1 Ki (average) | 2250 |
| | MC-1 EC₅₀ (average; cAMP HBL) | 332 |
| | MC-1 Eₘₐₓ (average; cAMP HBL) | 102% |
| 9.66 | Ac-Nle-cyclo(Glu-Pro-D-Phe-Arg-Dab)-Trp-NH₂ (SEQ ID NO: 69) | |
| | 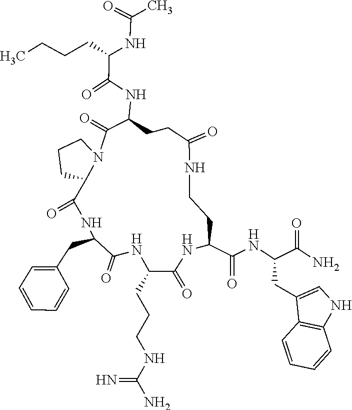 [SMILES: CCCC[C@H](NC(C)=O)C(=O)N[C@H]1CCC(=O)NCC[C@@H](C(=O)N[C@@H](Cc2c[nH]c3ccccc23)C(N)=O)NC(=O)[C@H](CCCNC(=N)N)NC(=O)[C@@H](Cc2ccccc2)NC(=O)[C@H]2CCCN2C1=O] | |
| | Assay | Result |
| | MC-4 Ki (average) | 185 |
| | MC-1 Ki (average) | 3 |
| | MC-1 EC₅₀ (average; cAMP HBL) | 0.18 |
| | MC-1 Eₘₐₓ (average; cAMP HBL) | 97% |

### 10. Examples of Multi-Particulate Formulations Utilized in the Invention

Formulations of the following described lots were made:

| | Amount (g) | | | | | | | | |
|---|---|---|---|---|---|---|---|---|---|
| Ingredient | Lot 22 | Lot 23 | Lot 24 | Lot 27 | Lot 31 | Lot 41 | Lot 40 | Lot 42 | Lot 49 |
| Eudragit L100-55 | | — | 49.5 | 35.5 | — | 23.25 | 19.00 | 19.8 | 23.25 |
| Eudragit S100 | | — | — | | 49.5 | 23.00 | 15.00 | 29.7 | 23.00 |
| Eudragit L100 | 9.9 | 49.5 | — | | — | — | 15.00 | — | — |
| Eudragit FS30D | — | — | — | 12.0 | — | 3.75 | — | — | 3.75 |
| Peptide 9.3 | 0.1 | 0.5 | 0.5 | 0.5 | 0.5 | 0.6 | 1.00 | 0.5 | — |
| Acetone | 70 | 350 | 350 | 350 | 350 | 350 | 350 | 350 | 350 |
| Water | — | — | — | — | 5 | 10 | 10 | 10 | — |
| Methanol | 5 | 20 | 20 | — | 10 | — | — | — | — |
| % Yield | 97.7 | 98.1 | 96.9 | 96.8 | 98.9 | 95.6 | 97.2 | 97.6 | 97.4 |

The various lots were loaded with between about 1% and 2% (w/w of peptide/polymer) of the cyclic peptide of Example 9.3. HPLC methods, employing a C-18 column, were employed for assays, including studies of cyclic peptide release in various acid and pH ranges. The drug load and encapsulation efficiency of the microparticles were determined after the manufacturing process. Performance of the microparticles was characterized by an in vitro release method.

Drug loading was determined by dissolving a known weight of the microparticles in an appropriate volume of phosphate buffer (1 L buffer containing 0.5 mL phosphoric acid with pH adjusted to 7.5 with sodium hydroxide) pH 7.5-8.0. The resulting solution was analyzed for the drug using HPLC. For encapsulation efficiency (EE), the microparticles were rinsed with 0.1 M HCl, dried and used as described for drug loading. Drug loading and EE values were calculated based on the drug and polymer starting weights. The drug loading was greater than 99% and encapsulation efficiency of all samples prepared was greater than 95%.

| Theoretical Drug | Actual | Encapsulation efficiency |
|---|---|---|
| Loading (g) | loading (g) | after rinsing (%) |
| 1.0 | 0.994 | 96.4% |
| 1.2 | 1.191 | 97.7% |
| 2.0 | 1.993 | 95.9% |

Dissolution of Eudragit® microparticles containing cyclic peptide of Example 9.3 was conducted using USP Apparatus 2 starting with 500 mL of the acid, which was 0.1 M HCl pH 1.2, or acetate buffer pH 4.5. About 1 g of the microparticles was accurately weighed and suspended in the acid at 37° C. for 2 hours. The pH of the medium was then sequentially adjusted to pH 5.5 for 1 hour, pH 6.8 for another hour and finally to pH 7.4 for 7 hours.

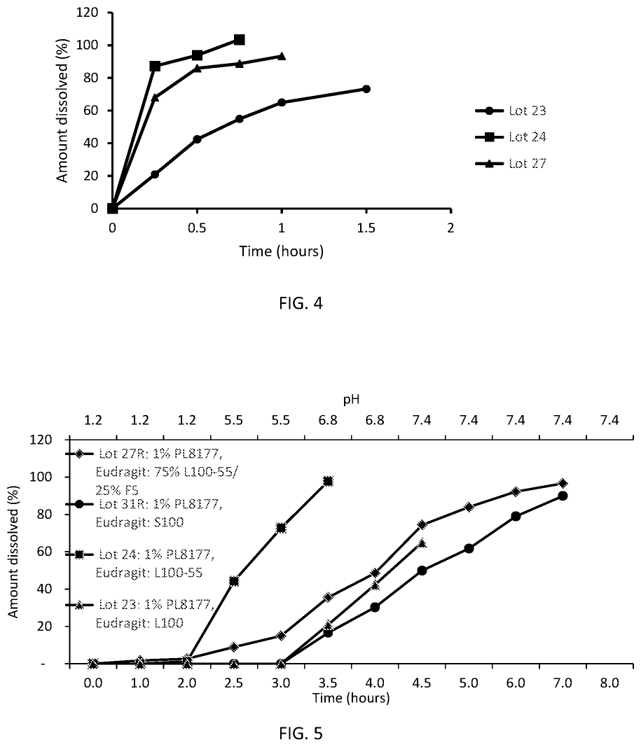

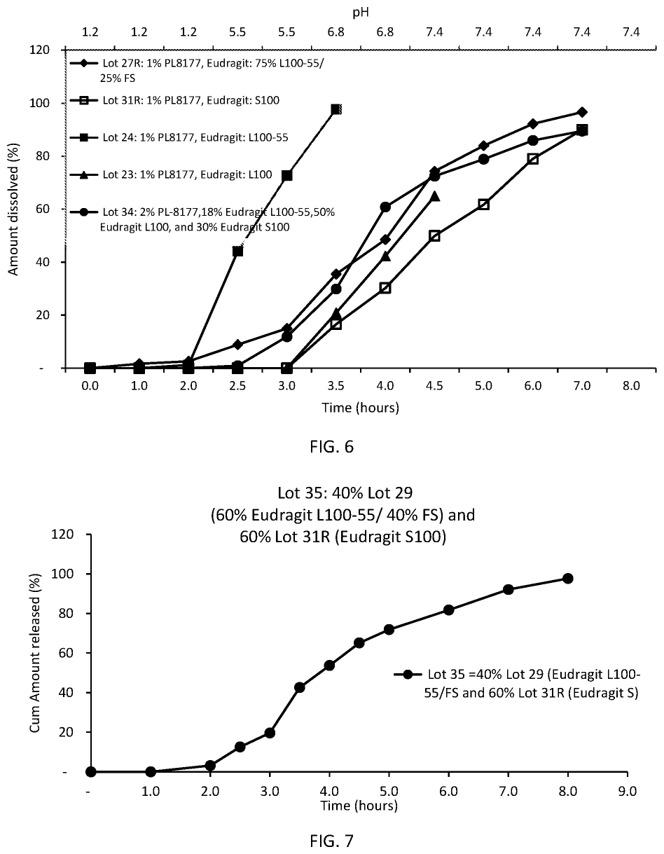

FIGS. 4-11 are representative release profiles of the cyclic peptide of Example 9.3 from microparticles prepared using various Eudragit® polymers and their blends. Generally, the release of the drug was pH dependent with the rate depending on the type of polymer used.

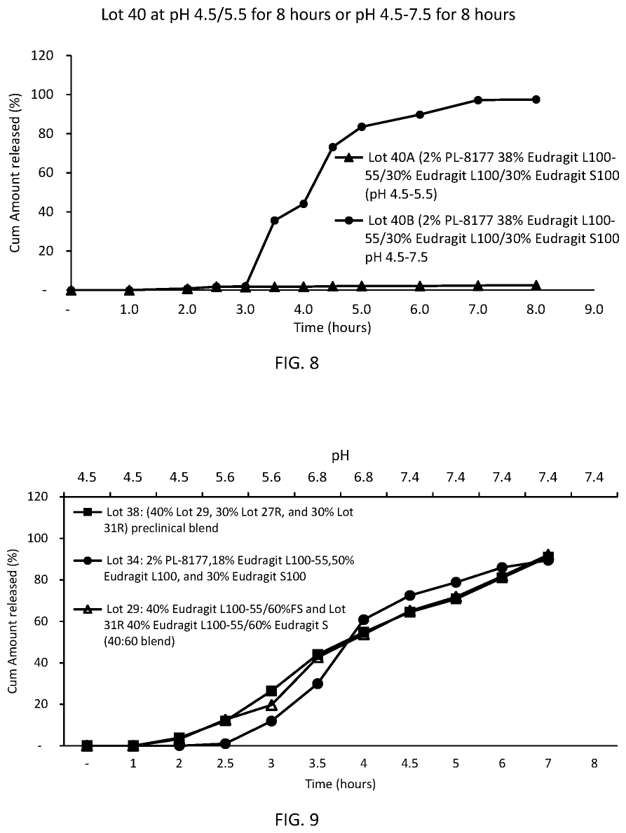

FIGS. 4-8 show release profiles of the cyclic peptide of Example 9.3 from microparticles prepared using the specified Eudragit® polymers and their blends. As is shown in FIG. 8, peptide release was pH dependent, with no release at pH 4.5-5.5, and approximately total release at pH 4.5-7.5.

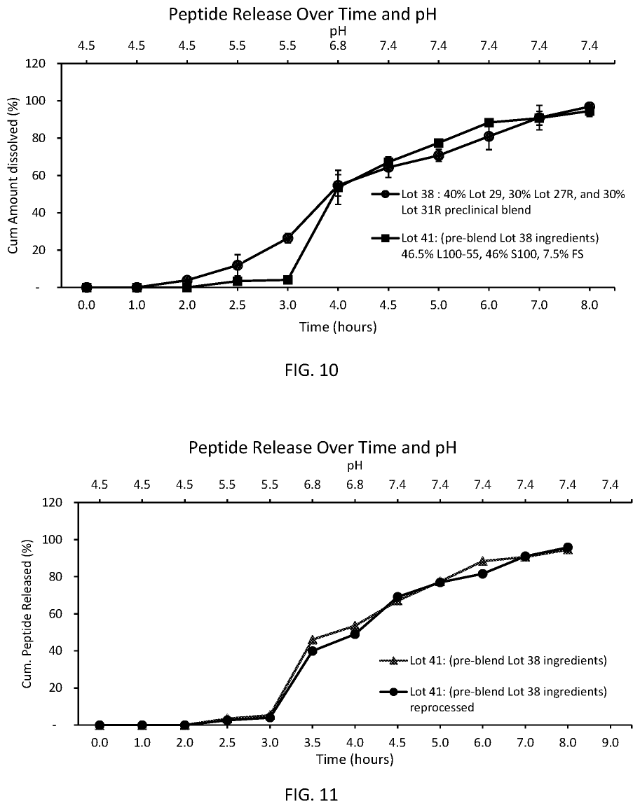

One product target profile was drug release throughout the GI tract starting with about 20% at pH 5.5. Blends of microparticles prepared using different polymers were tested. FIG. 9 shows the release profiles obtained from blended microparticles. Lot 38, comprising 40% of Lot 29, 30% of Lot 27 and 30% of Lot 31, was selected for further development. To simplify the preparation process and ensure blend uniformity, a lot of microparticles (Lot 41) was prepared by co-dissolving the polymers types in the same ratio as in the blended microparticles Lot 38 and used in preparing the microparticles, such that the formulation comprised about 46.5% Eudragit® L100-55, 46% Eudragit® S100 and 7.5% Eudragit® FS30D on a weight basis. FIG. 10 shows the release profiles of Lot 41 which was prepared from pre-blended polymers and is the same ratio as the Lot 38 microparticle blend. This lot was selected for evaluation in the preclinical pharmacokinetic and efficacy study. FIG. 11 shows a repeat dissolution profile for Lots 41 (n=3).

Placebo microparticles (Lot 49) containing the Eudragit® polymer blend as in Lot 41 were prepared and used as diluent for the active lot 41 and filled into preclinical rat capsule size 9. The placebo and the active microparticles were weighed and blended by geometric dilution. Blend uniformity testing was conducted and the microparticles were filled into preclinical capsules to contain 17 mg fill weight. Capsules containing 100, 50, 20 or 10 μg cyclic peptide of Example 9.3 strengths were prepared for testing in animal models. All the filled capsules were individually weighed, and the weights recorded.

### 11. Experimental Models

11.1 An evaluation of in vitro selectivity of the cyclic peptide of Example 9.3 compared with the endogenous MC1r agonists ACTH (adrenocorticotropic hormone) and α-MSH (alpha-melanocortin stimulating hormone) was conducted at Cerep in France. The results were as follows:

| | Functional — CEREP (EC₅₀; nM) | | | | |
|---|---|---|---|---|---|
| | MC1r | MC2r | MC3r | MC4r | MC5r |
| α-MSH | 4.47 | >10,000 | 9.8 | 10.8 | 560 |
| ACTH | 980 | 4.8 | 390 | 350 | 4100 |
| Example 9.3 | 0.57 | >10,000 | >10,000 | 510 | >10,000 |

11.2 In vitro activity and safety studies were conducted. The cyclic peptide of Example 9.3 demonstrated lipopolysaccharide-induced TNF-α inhibition comparable to α-MSH and ACTH. Separately, in a Eurofins lead profile, no activity was detected in any of 72 in vitro assays at 10 μM.

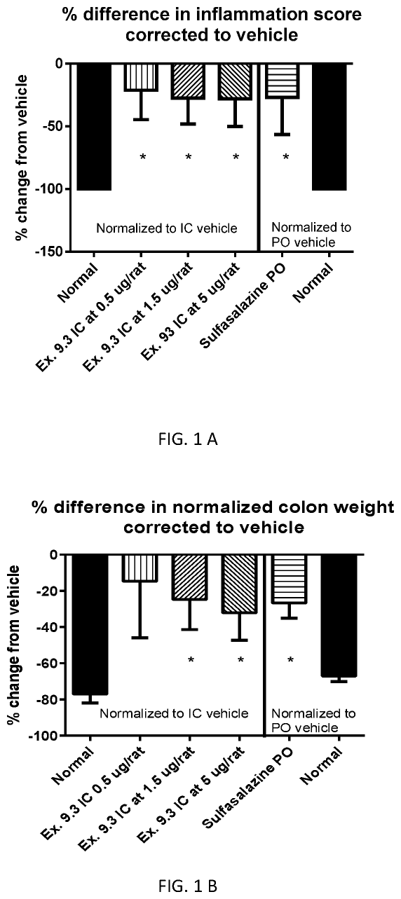

11.3 The cyclic peptide of Example 9.3 was evaluated in a cannulated rat model of bowel inflammation, in which dinitrobenzene sulfonic acid (DNBS) was administered rectally as a solution in male, 200 g Wistar rats to induce inflammation of the bowel lumen. The rats were implanted with a catheter in the proximal part of the ascending colon, which exited out the nape of the neck for dosing access. In groups of 10, the rats were dosed at: 0.5 μg and 5.0 μg cyclic peptide of Example 9.3 and vehicle (sterile water) via intracolonic injection at 24 h, 12 h, and 2 h before and 6 h after DNBS challenge, followed by twice-daily dosing for 5 consecutive days through day 7. Non-cannulated control rats were administered sulfasalazine (positive controls) and vehicle (untreated controls). As shown in FIG. 1A and FIG. 1B, in the DNBS rat model of bowel inflammation the cyclic peptide of Example 9.3 delivered to the lumen of the bowel was as active as sulfasalazine (standard of care), and superior to untreated controls, in reducing parameters of bowel inflammation (colon weight and inflammation score).

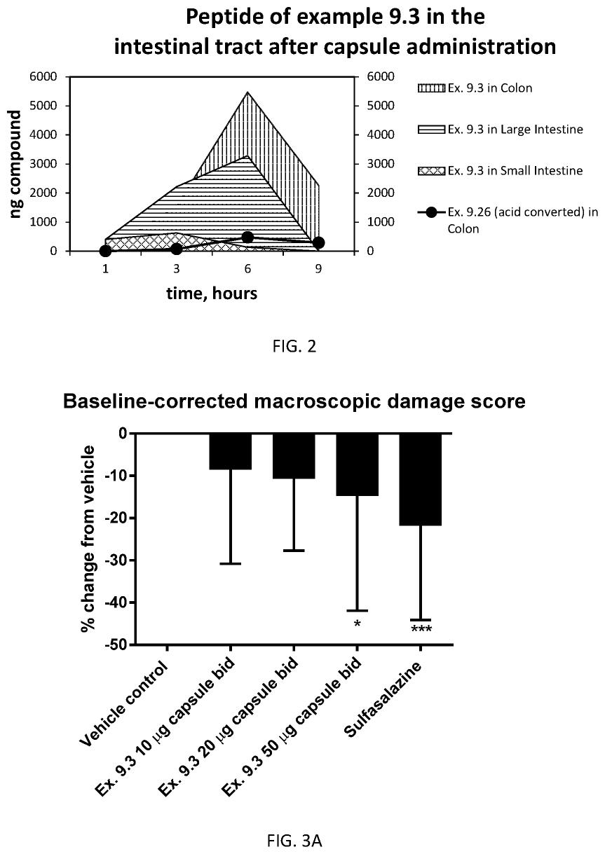

11.4 The pharmacokinetics and pharmacodynamics of an oral capsule formulation of Lot 41 of Section 10 above containing cyclic peptide of Example 9.3 for colon release was evaluated in rats. 24 total Sprague-Dawley rats weighing between 250-350 grams, 7-9 weeks old, were utilized, and were fasted overnight prior to oral dosing with a single capsule containing 0.1 mg of cyclic peptide of Example 9.3, with food and water ad libitum. Intestinal and colon contents were collected at specific time points (n=20) and after testing (n=4). As shown in FIG. 2, the oral formulation of cyclic peptide of Example 9.3 of Lot 41 was released in the colon and progressed through the rat intestinal tract in 9 hours.

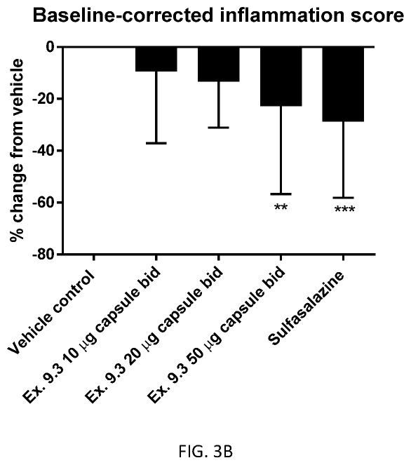

11.5 In a DNBS model of colitis in rats, an oral capsule formulation of Lot 41 containing 10 μg, 20 μg, and 50 μg of cyclic peptide of Example 9.3 was evaluated with twice daily (bid) administration, compared with placebo vehicle of lot 49 and sulfasalazine treatment. As shown in FIG. 3A and FIG. 3B, the baseline-corrected inflammation score and macroscopic damage score were both significantly lower (improved) with capsules containing 50 μg of cyclic peptide of Example 9.3 versus placebo vehicle, and to a similar degree as sulfasalazine. Assays of plasma did not detect any systemic cyclic peptide of Example 9.3.

### 12. Human Clinical Studies

An oral formulation of C₁₄-labeled cyclic peptide of Example 9.3 was formulated as for Lot 41. The C₁₄ label was used to evaluate the release and absorption of peptide of Example 9.3 in the distal GI tract following administration of a single oral dose. A combination of Eudragit® L100-55, Eudragit® S-100 and Eudragit® FS30D polymethacrylates were selected and utilized at a weight ratio of 23.25:23.0:12.5, where the weight of L100-55 and S-100 was dry weight of solid material, and the weight of FS30D was of a commercially prepared aqueous formulation wherein the 12.5 grams of liquid FS30D contained 3.75 grams polymer, for a weight ratio of polymer of 23.25:23:3.75. The combination of polymethacrylates were placed in acetone and stirred for an extended period. Appropriate quantities of C₁₄-labeled peptide of Example 9.3 was dissolved in water and mixed with the prepared acetone-polymethacrylates solution, stirred for an extended period, and dried under vacuum. The dried material was retrieved, diluted with additional dried polymetacrylate mixture not containing peptide to obtain the desired target concentration in a predetermined quantity of material, and milled to the desired diameter and sieved. The sieved material was placed within a gelatin size 2 capsule to provide an oral formulation.

The oral formulation was administered in a microdose level to 24 subjects, divided into six cohorts of 4 subjects each. Subjects in cohorts 1 through 5 received a laxative at 5, 8, 11, 14 and 17 hours post dose, and subjects in cohort 6 did not receive a laxative. Pharmacokinetic analyses were conducted of blood, urine and feces samples for subjects in all cohorts, including analysis for the presence of the peptide of Example 9.3 and a metabolite of the peptide of Example 9.3, the peptide of Example 9.26, with an N-terminal free acid.

The presence of the peptide of Example 9.26 provides evidence of the release of the peptide of Example 9.3 from the polymer matrix, since conversion of the C-terminal amide of Example example 9.3 to the acid of Example 9.26 can only occur subsequent to release of the peptide from the polymer matrix. The peptides of both Example 9.3 and Example 9.26 were found in significant and approximately equal levels in the analyzed fecal samples. In addition, no intact peptide of either Example 9.3 or Example 9.26 were found in plasma or urine. The only radioactive material identified in urine was the C₁₄ labelled phenylalanine. These results indicate significant protection of the drug product through the upper GI and delivery with subsequent release of drug product into the colon. Additionally, there was no drug product absorbed into the systemic circulation as evidenced by a lack of any detectable peptide of Example 9.3 or Example 9.26 in plasma or urine samples.

Although the invention has been described in detail with particular reference to these preferred embodiments, other embodiments can achieve the same results. Variations and modifications of the present invention will be obvious to those skilled in the art and it is intended to cover all such modifications and equivalents. The entire disclosures of all references, applications, patents, and publications cited above are hereby incorporated by reference.

We claim:

1. A lower gastrointestinal (GI) tract release pharmaceutical formulation comprising
a melanocortin receptor-specific peptide or a pharmaceutically acceptable salt thereof disposed within a particle matrix comprising at least one pH-dependent delayed release polymer comprising pH-sensitive methyl methacrylate/methacrylic copolymers selected from the group consisting of Eudragit® L100-55, Eudragit® S100 and Eudragit® FS30D.

2. (canceled)

3. The formulation of claim 1 wherein the peptide or pharmaceutically acceptable salt thereof is admixed within the particle matrix, thereby forming an admixture of the particle matrix and the peptide or pharmaceutically acceptable salt thereof.

4. The formulation of claim 3 wherein the admixture of the particle matrix and the peptide or pharmaceutically acceptable salt thereof is disposed within an aqueous soluble capsule or is formed into a tablet.

5. The formulation of claim 4 where the aqueous soluble capsule is a gelatin capsule.

6. (canceled)

7. The formulation of claim 4 wherein the capsule or tablet further comprises at least one of a seal coating and an enteric coating.

8. The formulation of claim 7 wherein the at least one of a seal coating and an enteric coating comprises a pH-dependent release polymer enteric coating.

9. (canceled)

10. (canceled)

11. The formulation of claim 1 wherein each of Eudragit® L100-55, Eudragit® S100 and Eudragit® FS30D are present in a weight-to-weight ratio of L100-55 to S100 to FS30D selected from the group consisting of about 6:6:1, or about 6.2:6.2:1 or about 23.25:23:3.75.

12. The formulation of claim 1 wherein the melanocortin receptor-specific peptide or a pharmaceutically acceptable salt thereof is a melanocortin-1 receptor (MC1r) specific peptide or a pharmaceutically acceptable salt thereof.

13. The formulation of claim 12 wherein the MC1r-specific peptide or a pharmaceutically acceptable salt thereof has a functional EC₅₀ value at the MC1r of less than about one nM.

14. The formulation of claim 13 wherein the MC1r-specific peptide or a pharmaceutically acceptable salt thereof has a functional EC₅₀ value at the melanocortin-4 receptor (MC4r) at least one hundred times the functional EC₅₀ value at MC1r.

15. (canceled)

16. The formulation of claim 12 wherein the MC1r-specific peptide or a pharmaceutically acceptable salt thereof is functionally inactive at the melanocortin-2 receptor (MC2r), the melanocortin-3 receptor (MC3r) and the melanocortin-5 receptor (MC5r).

17. The formulation of claim 12 wherein the delayed release polymer releases at least a portion of the MC1r-specific peptide or a pharmaceutically acceptable salt thereof in the colon.

18. The formulation of claim 12 wherein the delayed release polymer releases a therapeutically effective amount of the MC1r-specific peptide or a pharmaceutically acceptable salt thereof in the colon.

19. The formulation of claim 1 wherein the melanocortin receptor-specific peptide or a pharmaceutically acceptable salt thereof is Ac-Nle-cyclo(Glu-His-D-Phe-Arg-Dap)-Trp-NH₂ (SEQ ID NO:6) or a pharmaceutically acceptable salt thereof.

20. (canceled)

21. The formulation of claim 19 wherein the Eudragit® L100-55, Eudragit® S100 and Eudragit® FS30D are particles with a maximum particle size of no more than 1000 μm in diameter.

22. The formulation of claim 19 wherein the particles have a maximum particle size of no more than about 600 μm in diameter and a minimum particle size of at least about 250 μm in diameter.

23. The formulation of claim 19 wherein the percentage of Ac-Nle-cyclo(Glu-His-D-Phe-Arg-Dap)-Trp-NH₂ (SEQ ID NO:6) or a pharmaceutically acceptable salt thereof of delayed release polymer is no more than about 2% on a weight-to-weight basis.

24. The formulation of claim 23 wherein the percentage of Ac-Nle-cyclo(Glu-His-D-Phe-Arg-Dap)-Trp-NH₂ (SEQ ID NO:6) or a pharmaceutically acceptable salt thereof of delayed release polymer is no more than about 1% on a weight-to-weight basis.

25. The formulation of claim 19 wherein the percentage of Ac-Nle-cyclo(Glu-His-D-Phe-Arg-Dap)-Trp-NH₂ (SEQ ID NO:6) or a pharmaceutically acceptable salt thereof of delayed release polymer is no more than about 10% on a weight-to-weight basis.

26. The formulation of claim 19 further comprising at least one excipient selected from the group consisting of a surfactant, a disintegrant, a lubricant, and a binder.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)


SEQUENCE LISTING (69 sequences)

SEQ ID NO: 1 - protein, 5 aa, Artificial Sequence
  Derived from Homo sapiens sequence His-Phe-Arg-Trp
  MISC_FEATURE (4)..(4): Xaa can be any amino acid
HFRXW

SEQ ID NO: 2 - protein, 13 aa, Artificial
  Synthetic analog of SEQ ID NO2
  MOD_RES (1)..(1): ACETYLATION
  MOD_RES (13)..(13): AMIDATION
SYSMEHFRWGKPV

SEQ ID NO: 3 - protein, 13 aa, Homo sapiens
  MOD_RES (1)..(1): ACETYLATION
  MOD_RES (4)..(4): Nle
  MISC_FEATURE (7)..(7): D-Phe
  MOD_RES (13)..(13): AMIDATION
SYSXEHXRWGKPV

SEQ ID NO: 4 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MOD_RES (7)..(7): AMIDATION
XEHXRKW

SEQ ID NO: 5 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MOD_RES (7)..(7): AMIDATION
XEHXRXW

SEQ ID NO: 6 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dap
  MOD_RES (7)..(7): AMIDATION
XEHXRXW

SEQ ID NO: 7 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dap
  MOD_RES (7)..(7): AMIDATION
XDHXRXW

SEQ ID NO: 8 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge Through disulfide
  MISC_FEATURE (4)..(4): D-Phe
  MOD_RES (7)..(7): AMIDATION
XCHXRCW

SEQ ID NO: 9 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MOD_RES (6)..(6): Orn
  MOD_RES (7)..(7): AMIDATION
XEHXRXW

SEQ ID NO: 10 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MISC_FEATURE (7)..(7): D-Trp
  MOD_RES (7)..(7): AMIDATION
XEHXRXX

SEQ ID NO: 11 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (6)..(6): Dab
  MOD_RES (7)..(7): AMIDATION
XEHFRXW

SEQ ID NO: 12 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MISC_FEATURE (7)..(7): D-Nal 1
  MOD_RES (7)..(7): AMIDATION
XEHXRXX

SEQ ID NO: 13 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MISC_FEATURE (7)..(7): Nal 2
  MOD_RES (7)..(7): AMIDATION
XEHXRXX

SEQ ID NO: 14 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MISC_FEATURE (7)..(7): D-Nal 2
  MOD_RES (7)..(7): AMIDATION
XEHXRXX

SEQ ID NO: 15 - protein, 7 aa, Artificial Sequence
  Synthetic melanocortin-1 receptor-specific peptide of invention
  MOD_RES (1)..(1): ACETYLATION
  MISC_FEATURE (1)..(1): D-Phe
  MISC_FEATURE (2)..(6): Cyclic Bridge
  MISC_FEATURE (4)..(4): D-Phe
  MISC_FEATURE (6)..(6): Dab
  MOD_RES (7)..(7): AMIDATION
XEHXRXW

SEQ ID NO: 16 - protein, 7 aa, Artificial Sequence
  Synthetic melanocortin-1 receptor-specific peptide of invention
  MOD_RES (1)..(1): ACETYLATION
  MISC_FEATURE (2)..(6): Cyclic Bridge
  MISC_FEATURE (4)..(4): D-Phe
  MISC_FEATURE (6)..(6): Dab
  MOD_RES (7)..(7): AMIDATION
FEHXRXW

SEQ ID NO: 17 - protein, 6 aa, Artificial Sequence
  Synthetic melanocortin-1 receptor-specific peptide of invention
  MISC_FEATURE (1)..(1): Succinic acid
  MISC_FEATURE (1)..(5): Cyclic Bridge
  MISC_FEATURE (5)..(5): Dab
  MOD_RES (6)..(6): AMIDATION
XHFRXW

SEQ ID NO: 18 - protein, 6 aa, Artificial Sequence
  Synthetic melanocortin-1 receptor-specific peptide of invention
  MOD_RES (1)..(1): N-Terminal CH3-(CH2)2-C(=O)-
  MISC_FEATURE (1)..(5): Cyclic Bridge
  MISC_FEATURE (3)..(3): D-Phe
  MISC_FEATURE (5)..(5): Dab
  MOD_RES (6)..(6): AMIDATION
EHXRXW

SEQ ID NO: 19 - protein, 6 aa, Artificial Sequence
  Synthetic melanocortin-1 receptor-specific peptide of invention
  MOD_RES (1)..(1): N-Terminal CH3-(CH2)3-C(=O)-
  MISC_FEATURE (1)..(5): Cyclic Bridge
  MISC_FEATURE (3)..(3): D-Phe
  MISC_FEATURE (5)..(5): Dab
  MOD_RES (6)..(6): AMIDATION
EHXRXW

SEQ ID NO: 20 - protein, 6 aa, Artificial Sequence
  Synthetic melanocortin-1 receptor-specific peptide of invention
  MOD_RES (1)..(1): N-Terminal CH3-(CH2)4-C(=O)-
  MISC_FEATURE (1)..(5): Cyclic Bridge
  MISC_FEATURE (3)..(3): D-Phe
  MISC_FEATURE (5)..(5): Dab
  MOD_RES (6)..(6): AMIDATION
EHXRXW

SEQ ID NO: 21 - protein, 6 aa, Artificial Sequence
  Synthetic melanocortin-1 receptor-specific peptide of invention
  MOD_RES (1)..(1): N-Terminal CH3-(CH2)5-C(=O)-
  MISC_FEATURE (1)..(5): Cyclic Bridge
  MISC_FEATURE (3)..(3): D-Phe
  MISC_FEATURE (5)..(5): Dab
  MOD_RES (6)..(6): AMIDATION
EHXRXW

SEQ ID NO: 22 - protein, 6 aa, Artificial Sequence
  Synthetic melanocortin-1 receptor-specific peptide of invention
  MOD_RES (1)..(1): N-Terminal cyclo-propanoyl-
  MISC_FEATURE (1)..(5): Cyclic Bridge
  MISC_FEATURE (3)..(3): D-Phe
  MISC_FEATURE (5)..(5): Dab
  MOD_RES (6)..(6): AMIDATION
EHXRXW

SEQ ID NO: 23 - protein, 6 aa, Artificial Sequence
  Synthetic melanocortin-1 receptor-specific peptide of invention
  MOD_RES (1)..(1): N-Terminal cyclo-hexanoyl-
  MISC_FEATURE (1)..(5): Cyclic Bridge
  MISC_FEATURE (3)..(3): D-Phe
  MISC_FEATURE (5)..(5): Dab
  MOD_RES (6)..(6): AMIDATION
EHXRXW

SEQ ID NO: 24 - protein, 6 aa, Artificial Sequence
  Synthetic melanocortin-1 receptor-specific peptide of invention
  MOD_RES (1)..(1): N-Terminal cycopentyl acetyl-
  MISC_FEATURE (1)..(5): Cyclic Bridge
  MISC_FEATURE (3)..(3): D-Phe
  MISC_FEATURE (5)..(5): Dab
  MOD_RES (6)..(6): AMIDATION
EHXRXW

SEQ ID NO: 25 - protein, 6 aa, Artificial Sequence
  Synthetic melanocortin-1 receptor-specific peptide of invention
  MOD_RES (1)..(1): N-Terminal cyclohexyl acetyl-
  MISC_FEATURE (1)..(5): Cyclic Bridge
  MISC_FEATURE (3)..(3): D-Phe
  MISC_FEATURE (5)..(5): Dab
  MOD_RES (6)..(6): AMIDATION
EHXRXW

SEQ ID NO: 26 - protein, 6 aa, Artificial Sequence
  Synthetic melanocortin-1 receptor-specific peptide of invention
  MOD_RES (1)..(1): N-Terminal phenyl acetyl-
  MISC_FEATURE (1)..(5): Cyclic Bridge
  MISC_FEATURE (3)..(3): D-Phe
  MISC_FEATURE (5)..(5): Dab
  MOD_RES (6)..(6): AMIDATION
EHXRXW

SEQ ID NO: 27 - protein, 6 aa, Artificial Sequence
  Synthetic melanocortin-1 receptor-specific peptide of invention
  MOD_RES (1)..(1): N-Terminal phenyl propanoyl-
  MISC_FEATURE (1)..(5): Cyclic Bridge
  MISC_FEATURE (3)..(3): D-Phe
  MISC_FEATURE (5)..(5): Dab
  MOD_RES (6)..(6): AMIDATION
EHXRXW

SEQ ID NO: 28 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(7): Cyclic Bridge
  MISC_FEATURE (4)..(4): D-Phe
  MISC_FEATURE (6)..(6): Dab
  MOD_RES (7)..(7): AMIDATION
XEHXRXA

SEQ ID NO: 29 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dap
XEHXRXW

SEQ ID NO: 30 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(2): Dab
  MISC_FEATURE (2)..(6): Cyclic Bridge through -C(=O)-(CH2)2-C(=O)-
  MISC_FEATURE (4)..(4): D-Phe
  MISC_FEATURE (6)..(6): Dab
  MOD_RES (7)..(7): AMIDATION
XXHXRXW

SEQ ID NO: 31 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(2): Dap
  MISC_FEATURE (2)..(6): Cyclic Bridge Through -C(=O)-(CH2)2-C(=O)-
  MISC_FEATURE (4)..(4): D-Phe
  MISC_FEATURE (6)..(6): Dap
  MOD_RES (7)..(7): AMIDATION
XXHXRXW

SEQ ID NO: 32 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(2): Dab
  MISC_FEATURE (2)..(6): Cyclic Bridge Through -C(=O)-(CH2)2-C(C=O)-
  MISC_FEATURE (4)..(4): D-Phe
  MISC_FEATURE (6)..(6): Dap
  MOD_RES (7)..(7): AMIDATION
XXHXRXW

SEQ ID NO: 33 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MISC_FEATURE (7)..(7): Nal 1
  MOD_RES (7)..(7): AMIDATION
XEHXRXX

SEQ ID NO: 34 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
XEHXRXW

SEQ ID NO: 35 - protein, 6 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MOD_RES (6)..(6): AMIDATION
XEHXRX

SEQ ID NO: 36 - protein, 6 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MOD_RES (6)..(6): AMIDATION
XEHXAX

SEQ ID NO: 37 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MOD_RES (7)..(7): AMIDATION
XEHXGXW

SEQ ID NO: 38 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MOD_RES (7)..(7): AMIDATION
XEHXAXW

SEQ ID NO: 39 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MOD_RES (7)..(7): AMIDATION
XEAXRXW

SEQ ID NO: 40 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MOD_RES (7)..(7): AMIDATION
XERXRXW

SEQ ID NO: 41 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (3)..(3): Cit
  MISC_FEATURE (4)..(4): D-Phe
  MISC_FEATURE (6)..(6): Dab
  MOD_RES (7)..(7): AMIDATION
XEXXRXW

SEQ ID NO: 42 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MOD_RES (7)..(7): AMIDATION
XEHXRXK

SEQ ID NO: 43 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MOD_RES (7)..(7): AMIDATION
XEKXRXW

SEQ ID NO: 44 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (3)..(3): Dab
  MISC_FEATURE (4)..(4): D-Phe
  MISC_FEATURE (6)..(6): Dab
  MOD_RES (7)..(7): AMIDATION
XEXXRXW

SEQ ID NO: 45 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MOD_RES (3)..(3): Orn
  MISC_FEATURE (4)..(4): D-Phe
  MISC_FEATURE (6)..(6): Dab
  MOD_RES (7)..(7): AMIDATION
XEXXRXW

SEQ ID NO: 46 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(2): Dap
  MISC_FEATURE (2)..(6): Cyclic Bridge Through -C(=O)-(CH2)2-C(=O)-
  MISC_FEATURE (4)..(4): D-Phe
  MISC_FEATURE (6)..(6): Dab
  MOD_RES (7)..(7): AMIDATION
XXHXRXW

SEQ ID NO: 47 - protein, 6 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MOD_RES (6)..(6): AMIDATION
XEHXRX

SEQ ID NO: 48 - protein, 6 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MOD_RES (6)..(6): AMIDATION
XEHXAX

SEQ ID NO: 49 - protein, 5 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(5): Cyclic Bridge
  MISC_FEATURE (4)..(4): D-Phe
  MISC_FEATURE (5)..(5): Dab
  MOD_RES (5)..(5): AMIDATION
XEHXX

SEQ ID NO: 50 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MOD_RES (7)..(7): AMIDATION
XEHXGXW

SEQ ID NO: 51 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MOD_RES (7)..(7): AMIDATION
XEHXAXW

SEQ ID NO: 52 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MOD_RES (7)..(7): AMIDATION
XEAXRXW

SEQ ID NO: 53 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MOD_RES (7)..(7): AMIDATION
XERXRXW

SEQ ID NO: 54 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (3)..(3): Cit
  MISC_FEATURE (4)..(4): D-Phe
  MISC_FEATURE (6)..(6): Dab
  MOD_RES (7)..(7): AMIDATION
XEXXRXW

SEQ ID NO: 55 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MOD_RES (7)..(7): AMIDATION
XEHXRXK

SEQ ID NO: 56 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MOD_RES (7)..(7): AMIDATION
XEKXRXW

SEQ ID NO: 57 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (3)..(3): Dab
  MISC_FEATURE (4)..(4): D-Phe
  MISC_FEATURE (6)..(6): Dab
  MOD_RES (7)..(7): AMIDATION
XEXXRXW

SEQ ID NO: 58 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MOD_RES (3)..(3): Orn
  MISC_FEATURE (4)..(4): D-Phe
  MISC_FEATURE (6)..(6): Dab
  MOD_RES (7)..(7): AMIDATION
XEXXRXW

SEQ ID NO: 59 - protein, 6 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MOD_RES (3)..(3): Orn
  MISC_FEATURE (4)..(4): D-Phe
  MISC_FEATURE (6)..(6): Dab
  MOD_RES (6)..(6): AMIDATION
XEXXRX

SEQ ID NO: 60 - protein, 6 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MOD_RES (3)..(3): Orn
  MISC_FEATURE (4)..(4): D-Phe(2-Cl)
  MISC_FEATURE (6)..(6): Dab
  MOD_RES (6)..(6): AMIDATION
XEXXRX

SEQ ID NO: 61 - protein, 6 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MOD_RES (3)..(3): Orn
  MISC_FEATURE (4)..(4): D-Phe(3-Cl)
  MISC_FEATURE (6)..(6): Dab
  MOD_RES (6)..(6): AMIDATION
XEXXRX

SEQ ID NO: 62 - protein, 6 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MOD_RES (3)..(3): Orn
  MISC_FEATURE (4)..(4): D-Phe(4-Cl)
  MISC_FEATURE (6)..(6): Dab
  MOD_RES (6)..(6): AMIDATION
XEXXRX

SEQ ID NO: 63 - protein, 6 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MOD_RES (3)..(3): Orn
  MISC_FEATURE (4)..(4): D-Phe(2-F)
  MISC_FEATURE (6)..(6): Dab
  MOD_RES (6)..(6): AMIDATION
XEXXRX

SEQ ID NO: 64 - protein, 6 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MOD_RES (3)..(3): Orn
  MISC_FEATURE (4)..(4): D-Phe(4-F)
  MISC_FEATURE (6)..(6): Dab
  MOD_RES (6)..(6): AMIDATION
XEXXRX

SEQ ID NO: 65 - protein, 6 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MOD_RES (3)..(3): Orn
  MISC_FEATURE (4)..(4): D-Phe(3,4-F)
  MISC_FEATURE (6)..(6): Dab
  MOD_RES (6)..(6): AMIDATION
XEXXRX

SEQ ID NO: 66 - protein, 6 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MOD_RES (3)..(3): Orn
  MISC_FEATURE (4)..(4): D-Phe(4-Me)
  MISC_FEATURE (6)..(6): Dab
  MOD_RES (6)..(6): AMIDATION
XEXXRX

SEQ ID NO: 67 - protein, 6 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MOD_RES (3)..(3): Orn
  MISC_FEATURE (4)..(4): D-Phe(4-OMe)
  MISC_FEATURE (6)..(6): Dab
  MOD_RES (6)..(6): AMIDATION
XEXXRX

SEQ ID NO: 68 - protein, 6 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MOD_RES (6)..(6): AMIDATION
XEPXRX

SEQ ID NO: 69 - protein, 7 aa, Artificial Sequence
  Synthetic melanocortin-1 receptor-specific peptide of invention
  MOD_RES (1)..(1): ACETYLATION
  MOD_RES (1)..(1): Nle
  MISC_FEATURE (2)..(6): Cyclic Bridge
  MISC_FEATURE (4)..(4): D-Phe
  MISC_FEATURE (6)..(6): Dab
  MOD_RES (7)..(7): AMIDATION
XEPXRXW